(19) United States
(12) Patent Application Publication
(10) Pub. No.: US 2024/0325536 A1
(43) Pub. Date: Oct. 3, 2024

(54) TRANSPLANTED CELL PROTECTION VIA MODIFIED Fc RECEPTORS

(71) Applicant: THE REGENTS OF THE UNIVERSITY OF CALIFORNIA, Oakland, CA (US)

(72) Inventor: Tobias Deuse, Burlingame, CA (US)

(73) Assignee: THE REGENTS OF THE UNIVERSITY OF CALIFORNIA, Oakland, CA (US)

(21) Appl. No.: 18/281,532

(22) PCT Filed: Mar. 29, 2022
(86) PCT No.: PCT/US2022/022368

§ 371 Date: Sep. 11, 2023

Related U.S. Application Data
(60) Provisional application No. 63/168,225, filed on Mar. 30, 2021.
(60) Provisional application No. 63/305,587, filed on Feb. 1, 2022.

(51) Int. Cl. A61K 39/00; A61K 35/36; A61K 35/39; C07K 14/725; C07K 14/735; C07K 16/28

(52) U.S. Cl. CPC A61K 39/464412; A61K 35/36; A61K 35/39; A61K 39/4611; A61K 39/4621; A61K 39/4631; C07K 14/7051; C07K 14/70535; C07K 16/2803; A61K 2239/26; C07K 2317/622

(57) ABSTRACT

The invention provides, for the first time, cells that express truncated or modified Fc Receptor proteins (e.g. CD16t, CD32t, or CD64t) to evade antibody-dependent cellular cytotoxicity (ADCC) or complement-dependent cytotoxicity (CDC). The cells may be pluripotent cells, including hypoimmune pluripotent cells (HIP) or ABO blood type O Rhesus Factor negative HIP cells (HIPO−), that express a truncated or modified Fc Receptor. The invention encompasses cells derived from the pluripotent cells as well as primary cells. The cells may also be differentiated cells, including chimeric antigen receptor (CAR) cells, T cells, natural killer (NK) cells, endothelial cells, dopaminergic neurons, neuroglial cells, pancreatic islet cells, pancreatic beta cells, thyroid cells, fibroblasts, hepatocytes, cardiomyocytes, or retinal pigment endothelium cells.

### I. CROSS REFERENCE TO RELATED APPLICATIONS

This application claims priority under 35 U.S.C. § 119(e) to U.S. Provisional Application Nos. 63/168,225, filed Mar. 30, 2021, and 63/305,587, filed Feb. 1, 2022 and are incorporated herein by reference in their entirety.

### FIELD OF THE INVENTION

The invention relates to regenerative or oncology cell therapies. In some embodiments, the regenerative cell therapy comprises transplanting cells or cell lines into patients in need thereof. In some embodiments, the cell lines comprise pluripotent cells that express a cell-surface Fc receptor that has been truncated to remove the intracellular signalling domain. In other embodiments, the cell-surface Fc receptor is a truncated CD16, CD32, or CD64 protein (CD16t, CD32t, or CD64t, respectively). In other embodiments, they are hypoimmunogenic, have an O blood type, or are Rh factor negative. In other embodiments, the regenerative or oncology cell therapy products show prolonged survival in an allogeneic recipient. In some embodiments, the regenerative cell therapy is used in the treatment of injured organs and tissue, the immune oncology cell product is used to treat cancer. In some embodiments, the regenerative cell therapy of the invention utilizes pancreatic islet cells, thyroid cells, hepatocytes, chimeric antigen receptor (CAR) cells, endothelial cells, dopaminergic neurons, neuroglial cells, cardiomyocytes, or retinal pigment endothelium cells used for treating diseases or rehabilitating damaged tissues. In other embodiments, the immune oncology cell product of the invention utilizes T cells, natural killer (NK) cells, or other immune cells like innate lymphoid cells (ILCs).

### II. BACKGROUND OF THE INVENTION

Regenerative cell therapy is an important potential treatment for regenerating injured organs and tissue. With the low availability of organs for transplantation and the accompanying lengthy wait, the possibility of regenerating tissue by transplanting readily available cell lines into patients is understandably appealing. Regenerative cell therapy has shown promising initial results for rehabilitating damaged tissues after transplantation in animal models (e.g. after myocardial infarction). The propensity for the transplant recipient's immune system to reject allogeneic material, however, greatly reduces the potential efficacy of therapeutics and diminishes the possible positive effects surrounding such treatments.

Although chimeric antigen receptor (CAR) T cell therapy has made remarkable strides in the treatment of patients with difficult to treat cancers, strategies must be developed to benefit great numbers of individuals with solid tumors. Several barriers need to be overcome before this type of therapy becomes a widely accepted standard treatment for different cancers. Among the barriers, poor persistence of infused cells is a critical challenge in successful cancer therapy. It has been well-recognized that poor persistence of infused CAR T cells is inversely correlated with durable clinical remissions in patients with cancers and limited CAR T cell persistence has been linked to the immunogenicity of the engineered T cell product (Korde, N., et al. A phase II trial of pan-KIR2D blockade with IPH2101 in smoldering multiple myeloma. Haematologica 99, e81-83 (2014), Schmidts, A. & Maus, M. V. Making CAR T Cells a Solid Option for Solid Tumors. Front Immunol 9, 2593 (2018)). In some cases of autologous CAR T therapy, the specific immune response has been identified to be directed against the CAR itself and eliminates the cell therapeutic before its action is fully achieved (Hege, K. M., et al. Safety, tumor trafficking and immunogenicity of chimeric antigen receptor (CAR)-T cells specific for TAG-72 in colorectal cancer. J Immunother Cancer 5, 22 (2017), Lamers, C. H., et al. Treatment of metastatic renal cell carcinoma with CAIX CAR-engineered T cells: clinical evaluation and management of on-target toxicity. Mol Ther 21, 904-912 (2013)). CD19 expression is maintained in B-lineage cells that have undergone neoplastic transformation, and therefore, CD19 is useful in the diagnosis of B cell leukemia and as a target for CAR T cell therapy. Modem leukemia therapy includes cytotoxic pre-conditioning followed by administration of anti-CD19 CAR T cells, which target all B cells including the leukemic and benign populations. Therefore, the host-versus-graft immune response in patients treated for B cell leukemia is largely blunted and shows long-lasting suppression of antibody production. CAR T cells are thus better protected from immune rejection when treating leukemia patients. In solid organ cancer patients, the immune response against the CAR T product is enhanced (Hege, K. M., et al. Safety, tumor trafficking and immunogenicity of chimeric antigen receptor (CAR)-T cells specific for TAG-72 in colorectal cancer. J Immunother Cancer 5, 22 (2017)) and minimizes the efficacy of this therapy. Poor persistence hinders the effector functions of the infused cells and hampers the long-term therapeutic success.

Autologous induced pluripotent stem cells (iPSCs) theoretically constitute an unlimited cell source for patient-specific cell-based organ repair strategies. Their generation, however, poses technical and manufacturing challenges and is a lengthy process that conceptually prevents any acute treatment modalities. Allogeneic iPSC-based therapies or embryonic stem cell-based therapies are easier from a manufacturing standpoint and allow the generation of well-screened, standardized, high-quality cell products. Because of their allogeneic origin, however, such cell products would undergo rejection. With the reduction or elimination of the cells' antigenicity, universally-acceptable cell products could be produced. Because pluripotent stem cells can be differentiated into any cell type of the three germ layers, the potential application of stem cell therapy is wide-ranging. Differentiation can be performed ex vivo or in vivo by transplanting progenitor cells that continue to differentiate and mature in the organ environment of the implantation site. Ex vivo differentiation allows researchers or clinicians to closely monitor the procedure and ensures that the proper population of cells is generated prior to transplantation.

In most cases, however, undifferentiated pluripotent stem cells are avoided in clinical transplant therapies due to their propensity to form teratomas. Rather, such therapies tend to use differentiated cells (e.g. stem cell-derived cardiomyocytes transplanted into the myocardium of patients suffering from heart failure). Clinical applications of such pluripotent cells or tissues would benefit from a “safety feature” that controls the growth and survival of cells after their transplantation.

The art seeks stem cells capable of producing cells that are used to regenerate or replace diseased or deficient cells. Pluripotent stem cells (PSCs) may be used because they rapidly propagate and differentiate into many possible cell types. The family of PSCs includes several members generated via different techniques and possessing distinct immunogenic features. Patient compatibility with engineered cells or tissues derived from PSCs determines the risk of immune rejection and the requirement for immunosuppression.

Embryonic stem cells (ESCs) isolated from the inner cell mass of blastocysts exhibit the histocompatibility antigens that are mismatches with recipients. This immunological barrier cannot be solved by human leukocyte antigen (HLA)-typed banks of ESCs because even HLA-matched PSC grafts undergo rejection because of mismatches in non-HLA molecules that function as minor antigens. This is also true for allogeneic induced pluripotent stem cells (iPSCs).

Hypoimmunogenic pluripotent (HIP) cells and cell products have gene knockouts or transgenes to protect them from the cellular components of the immune system that include T cells, NK cells, and macrophages. They may also be ABO blood group type O and Rh negative (HIPO−).

Immune rejection presents the principal hurdle for the success of cell therapeutics and much effort is currently devoted to developing universal allogeneic off-the-shelf cells evading cellular rejection (Yoshihara, E. et al. Nature 586, 606-611 (2020); Wang, B. et al. Nat Biomed Eng 5, 429-440 (2021); Deuse, T. et al. Proc Natl Acad Sci USA 118, (2021)). Such gene-edited hypoimmune cells, however, remain susceptible to antibody killing directed against non-HLA epitopes, cell-type specific autoantigens, as well as xenogeneic (Klee, G. G. Arch Pathol Lab Med 124, 921-923 (2000)) or synthetic constructs (Choe, J. H. et al. Sci Transl Med 13 (2021)) in engineered cells and viral products. Such cells result from the transduction process (Lamers, C. H. et al. Blood 117, 72-82 (2011); Jensen, M. C. et al. Biol Bloo dMarrow Transplant 16, 1245-1256 (2010)). Cytotoxic antibodies can be pre-existing or treatment-induced (Wagner, D. L. et al. Nat Rev Clin Oncol 18, 379-393 (2021)) and jeopardize the persistence and efficacy of the cell therapeutics.

The two most relevant killing mechanisms involving antibodies are antibody-dependent cellular cytotoxicity (ADCC) by Natural Killer (NK) cells, macrophages, B-cells or granulocytes and complement-dependent cytotoxicity (CDC) via activation of the complement cascade. All of those killing mechanisms utilize antibodies that bind to a target cell and activate the effector immune cells or complement (FIG. 1). IgG antibodies can be potent mediations of both ADCC and CDC. IgG antibodies have two variable Fab regions which bind to specific epitopes. The crystalizable Fc region sticks out and serves for the binding of NK cells, B-cells, macrophages, granulocytes, or complement.

Receptors that recognize the Fc portion of IgG are divided into four different classes: FcγRI (CD64), FcγRII (CD32), FcγRIII (CD16), and FcγRIV. Whereas FcγRI displays high affinity for the antibody constant region and restricted isotype specificity, FcγRII and FcγRIII have low affinity for the Fc region of IgG but a broader isotype binding pattern, and FcγRIV is a recently identified receptor with intermediate affinity and restricted subclass specificity. Physiologically, FcγRI functions during early immune responses, while FcγRII and RIII recognize IgG as aggregates surrounding multivalent antigens during late immune responses.

If an antibody binds to an unprotected cell via its Fab regions, the Fc can be bound by NK cells (mostly via their CD16 receptor), macrophages (mostly via CD64, CD16 or CD32), B-cells (mostly via CD32), or granulocytes (mostly via CD32, CD16, or CD64) and mediate ADCC. Complement can also bind to Fc and activate its cascade and form the membrane attack complex (MAC) for CDC killing.

### III. SUMMARY OF THE INVENTION

The invention provides cells that express a cell-surface Fc receptor that has been truncated or modified to remove the intracellular signalling function or the intracellular signalling domain. The cells may be transplanted into patients in need thereof. In some embodiments, the regenerative or oncology cell therapy of the invention utilizes pancreatic islet cells, thyroid cells, chimeric antigen receptor (CAR) cells, T cells, NK cells, ILCs, hepatocytes, endothelial cells, dopaminergic neurons, neuroglial cells, cardiomyocytes, or retinal pigment endothelium cells used for treating cancer, diseases or rehabilitating damaged tissues.

The invention provides cells expressing a truncated CD16, CD32, or CD64 (FIG. 2) that sequesters the Fc portion of local antibodies and thus inhibits ADCC and CDC. (Compare FIGS. 1 and 3). The cells may be primary cells, differentiated cells, pluripotent cells, including hypoimmune pluripotent cells (HIP), ABO blood type O Rhesus Factor negative HIP cells (HIPO−) induced pluripotent stem cells (iPSC), iPSCs that are O−, embryonic stem cells (ESC), or ESCs that are O−, any of which further comprise the truncated CD64 expression. Primary cells are isolated directly from tissues.

In other embodiments, the cells may be pancreatic islet cells, thyroid cells, chimeric antigen receptor (CAR) cells, T cells, NK cells, ILCs, endothelial cells, hepatocyte, dopaminergic neurons, neuroglial cells, cardiomyocytes, or retinal pigment endothelium cells used for treating diseases or rehabilitating damaged tissues.

CD64 is constitutively found only on macrophages and monocytes and is usually not expressed on tissue cells. It is more commonly known as Fc-gamma receptor 1 (FcγRI) and binds IgG Fc regions with high affinity. CD64 overexpression on target cells sequesters the IgG Fc and binds it to the target cell. In cells that have no intracellular pathways for cellular activation via CD64 there may be no functional effect on such cells. For all cells that allow the cytoplasmic tail of CD64 to induce intracellular activation pathways, Fc binding would affect the cell and might perturbate its physiology. This can be avoided by truncating or modifying the intracellular signaling domain of Fc receptors such as CD64. Fc receptors with no intracellular domains will not trigger cell activation but are still able to sequester IgG Fc. This is true for CD64, CD32, and CD16. Even if the free Fab regions would bind neighboring target cells, the occupation of the Fc prevents any ADCC or CDC.

Thus, the invention provides a modified cell, wherein the modified cell expresses an Fc receptor protein comprising a truncation or modification, wherein the Fc receptor protein expression causes the modified cell to be less susceptible to antibody dependent cellular cytoxicity (ADCC) or complement-dependent cytotoxicity (CDC), wherein the truncation or modification reduces or eliminates intracellular signalling. In some aspects of the invention, the Fc receptor protein is selected from the group consisting of a truncated CD16 (CD16t), truncated CD32 (CD32t), and truncated CD64 (CD64t). In other aspects, the cell is a primary cell, differentiated cell, or pluripotent cell.

In some aspects of the invention, the Fc receptor protein is selected from the group consisting of CD64t protein having at least a 90% sequence identity to SEQ ID NO:16, CD16t protein having at least a 90% sequence identity to SEQ ID NO:14, and CD32t protein having at least a 90% sequence identity to SEQ ID NO:15. In a preferred aspect, the CD64t protein has the sequence of SEQ ID NO: 16.

In some aspects of the invention, the modified cell is derived from a human hypo-immunogenic pluripotent (HIP) cell, a human hypo-immunogenic pluripotent ABO blood group O Rhesus Factor negative (HIPO−) cell, a human induced pluripotent stem cell (iPSC), or a human embryonic stem cell (ESC).

In other aspects, the modified cell is from a species that is selected from the group consisting of a human, monkey, cow, pig, chicken, turkey, horse, sheep, goat, donkey, mule, duck, goose, buffalo, camel, yak, llama, alpaca, mouse, rat, dog, cat, hamster, and guinea pig.

The invention provides the modified cells as disclosed herein further comprising a suicide gene that is activated by a trigger that causes the modified cell to die. In some aspects, the suicide gene is a herpes simplex virus thymidine kinase gene (HSV-tk) and the trigger is ganciclovir. In a preferred aspect, the HSV-tk gene encodes a protein comprising at least a 90% sequence identity to SEQ ID NO:4. In another preferred aspect, the HSV-tk gene encodes a protein comprising the sequence of SEQ ID NO:4. In another aspect, the suicide gene is an Escherichia coli cytosine deaminase gene (EC-CD) and the trigger is 5-fluorocytosine (5-FC). In a preferred aspect, the EC-CD gene encodes a protein comprising at least a 90% sequence identity to SEQ ID NO:5. In another preferred aspect, the EC-CD gene encodes a protein comprising the sequence of SEQ ID NO:5. In another aspect, the suicide gene encodes an inducible Caspase protein and the trigger is a chemical inducer of dimerization (CID). In a preferred spect, the suicide gene encodes an inducible Caspase protein comprising at least a 90% sequence identity to SEQ ID NO:6. In another preffered aspect, the suicide gene encodes an inducible Caspase protein comprising the sequence of SEQ ID NO:6. In another aspect, the CID is AP1903.

The invention provides that the modified cells as disclosed herein are selected from the group consisting of a chimeric antigen receptor (CAR) cell, a T cell, an NK cell, an ILC, an endothelial cell, a dopaminergic neuron, a pancreatic islet cell, a pancreatic beta cell, a thyroid cell, a fibroblast, a hepatocyte, a cardiomyocyte, and a retinal pigment endothelium cell. In some aspects, the cell is a CAR-T or a CAR-NK cell.

The invention provides a method comprising transplanting the cells as disclosed herein into a subject, wherein the subject is a human, monkey, cow, pig, chicken, turkey, horse, sheep, goat, donkey, mule, duck, goose, buffalo, camel, yak, llama, alpaca, mouse, rat, dog, cat, hamster, guinea pig. In some aspects of the method, the modified cell is selected from the group consisting of a chimeric antigen receptor (CAR) cell, a T cell, an NK cell, an ILC, an endothelial cell, a dopaminergic neuron, a pancreatic islet cell, a cardiomyocyte, and a retinal pigment endothelium cell.

The invention provides a method of treating a disease, comprising administering the cells disclosed herein, or cells derived therefrom, to a subject. In some aspects of the method, the cell or the derivative cell is selected from the group consisting of a chimeric antigen receptor (CAR) cell, a T cell, an NK cell, an ILC, an endothelial cell, a dopaminergic neuron, a pancreatic islet cell, a pancreatic beta cell, a thyroid cell, a fibroblast, a hepatocyte, a cardiomyocyte, and a retinal pigment endothelium cell. In other aspects, the disease is selected from the group consisting of Type I Diabetes, a cardiac disease, a neurological disease, an endocrine disease, a cancer, an ocular disease, and a vascular disease.

The invention provides a method for generating the modified cell as disclosed herein comprising expressing the CD16t, CD32t, or CD64t protein in a parental non-modified version of the cell. In some aspects, the modified cell has a human, monkey, cow, pig, chicken, turkey, horse, sheep, goat, donkey, mule, duck, goose, buffalo, camel, yak, llama, alpaca, mouse, rat, dog, cat, hamster, or guinea pig origin. In other aspects, the modified cell is derived from a HIP cell, a HIPO− cell, an iPSC cel, or an ESC cell.

In some aspects of the method, the CD16t, CD32t, or CD64t expression results from introducing at least one copy of a human CD16t, CD32t, or CD64t gene under the control of a promoter into the parental version of the modified cell. In a preferred aspect, the promoter is a constitutive promoter.

The invention provides a pharmaceutical composition for treating a disease comprising a modified cell or a cell derived therefrom as disclosed herein and a pharmaceutically-acceptable carrier. In some aspects, the cell is selected from the group consisting of a chimeric antigen receptor (CAR) cell, a T cell, an NK cell, an ILC, an endothelial cell, a dopaminergic neuron, a neuroglial cell, a pancreatic islet cell, a pancreatic beta cell, a thyroid cell, another endocrine cell, a fibroblast, a hepatocyte, a cardiomyocyte, and a retinal pigment endothelium cell. In other aspects, the disease is selected from the group consisting of Type I Diabetes, a cardiac disease, a neurological disease, an endocrine disease, a cancer, an ocular disease, and a vascular disease.

The invention provides a medicament for treating a disease, comprising a cell as described herein or one derived from a modified cell as described herein. In some aspects, the cell or derivative cell is selected from the group consisting of a chimeric antigen receptor (CAR) cell, a T cell, an NK cell, an ILC, an endothelial cell, a dopaminergic neuron, a neuroglial cell, a pancreatic islet cell, a pancreatic beta cell, a thyroid cell, a fibroblast, a hepatocyte, a cardiomyocyte, and a retinal pigment endothelium cell. In other aspects, the disease is selected from the group consisting of Type I Diabetes, a cardiac disease, a neurological disease, an endocrine disease, a cancer, an ocular disease, and a vascular disease.

The invention provides a modified cell, comprising a CD16t, CD32t, or CD64t protein, wherein the protein expression causes the modified cell to be less susceptible to antibody dependent cellular cytoxicity (ADCC) or complement-dependent cytotoxicity (CDC). In some aspects, the modified cell comprises enhanced CD16t, CD32t, or CD64t protein levels resulting from genetic engineering. In other aspects, the cell is selected from the group consisting of a chimeric antigen receptor (CAR) cell, a T cell, an NK cell, an ILC, an endothelial cell, a dopaminergic neuron, a pancreatic islet cell, a pancreatic beta cell, a thyroid cell, a fibroblast, a hepatocyte, a cardiomyocyte, and a retinal pigment endothelium cell.

The invention provides a modified cell as disclosed herein wherein said cell comprises an Fc receptor chimera comprising a cytoplasmic domain that does not mediate an Fc receptor signalling pathway and wherein said cytoplasmic domain promotes endocytosis of an antibody bound by its Fc to an extracellular domain of said Fc receptor chimera. In some aspects, the cytoplasmic domain is from a transferrin receptor. In other aspects, the transferrin receptor is TfR1 or TfR2. In preferred aspects, the Fc receptor chimera comprises a CD16, CD32, or CD64 cell surface domain and a TfR1 or TfR2 cytoplasmic domain.

The invention provides modified cells as disclosed herein, wherein the cells express a recombinant SIRPα engager protein. In other aspects, the SIRPα engager protein comprises an immunoglobulin superfamily domain, an antibody Fab domain, or a single chain variable fragment (scFV). In other aspects, the SIRPα engager protein binds to SIRPα with an affinity measured by its dissociation constant (Kd), wherein the Kd is between about 10⁻⁷ and 10⁻¹³ M.

In some aspects, the modified cells as disclosed herein comprise a B2M−/− phenotype, a CIITA−/− phenotype, a CD64t, and a SIRPα-engager molecule. In other aspects, the SIRPα-engager protein is CD47. In other aspects, the cell is an engineered NK cell.

### IV. BRIEF DESCRIPTION OF THE DRAWINGS

FIG. 1 is a schematic diagram of ADCC and CDC. When an antibody of the IgG type finds an antigen on the surface of a target islet cell, it activates NK cells or complement to kill the target cell via antibody-dependent cellular cytotoxicity (ADCC) or complement-dependent cytotoxicity (CDC).

FIG. 2 is a schematic diagram of an aspect of the invention that utilizes an engineered Fc receptor that sequesters antibodies without alterating the target cell physiology.

A truncated or modified CD16, CD32, or CD64 coding sequence (CDS) lacks a functioning intracellular signaling domain. The protein domains according to Uniprot are shown. The cytoplasmic domains for truncation or modification are indicated.

FIG. 3 shows a truncated CD64 (CD64t) capturing free IgG Fc without inducing any signaling in the target cell.

FIGS. 4A and 4B. FIG. 4A shows wild-type (wt) human iECs epressing full-length CD64. FIG. 4B shows these iECs capturing alemtuzumab Fc in a concentration-dependent manner.

FIGS. 5A and 5B. Wild-type (wt) human iECs epressing CD64t similarly captured alemtuzumab Fc in a concentration-dependent manner. FIG. 5A shows CD64t expression on wt iECs. FIG. 5B shows the iECs capturing alemtuzumab Fc in a concentration-dependent manner.

FIGS. 6A and 6B show a flow cytometry histogram for CD64 expression on human HIP iECs (FIG. 6A). Binding of free IgG1 F_{c} was assessed using alemtuzumab, an anti-CD52 antibody without specific binding site on the HIP iECs. There was no IgG1 F_{c} binding over the whole concentration range tested (FIG. 6B).

FIGS. 7A and 7B show a flow cytometry histogram for CD64 expression on human HIP iECs transduced to express a CD64 transgene (HIP iECs^{CD64}, FIG. 7A). Binding of free IgG1 F_{c} was assessed using alemtuzumab, an anti-CD52 antibody without specific binding site on the HIP iECs^{CD64}. There was concentration-dependent binding of IgG1 (FIG. 7B). Since there are no Fab binding epitopes, this binding must be via F_{c} and CD64.

FIGS. 8A and 8B show a flow cytometry histogram for CD64t expression on human HIP iECs transduced to express a CD64t transgene (HIP iECs^{CD64t}, FIG. 8A). There was concentration-dependent binding of IgG1 (FIG. 8B). Since there are no Fab binding epitopes, this binding must be via F_{c} and CD64t.

FIG. 9 shows HIP iECs transduced to express CD52, the target epitope for the human anti-CD52 IgG1 antibody alemtuzumab. HIP iEC^{CD52} were challenged in impedance NK cell ADCC assays with different alemtuzumab concentrations and were killed in a concentration-dependent fashion (mean±SD, three independent replicates per group and time point).

FIG. 10 shows HIP iEC^{CD52} challenged in impedance CDC assays with different alemtuzumab concentrations. HIP iEC^{CD52} were killed in a concentration-dependent fashion (mean±SD, three independent replicates per group and time point).

FIG. 11 shows HIP iECs transduced to express CD52, the target epitope for the human anti-CD52 IgG1 antibody alemtuzumab as well as CD64t. HIP iEC^{CD52,CD64t} were challenged in impedance NK cell ADCC assays with different alemtuzumab concentrations and were completely resistant against killing (mean±SD, three independent replicates per group and time point).

FIG. 12 shows HIP iEC^{CD52,CD64t} challenged in impedance CDC assays with different alemtuzumab concentrations. HIP iEC^{CD52,CD64t} were completely resistant against killing (mean±SD, three independent replicates per group and time point).

FIGS. 13A and 13B show representative flow cytometry histograms for thyroid peroxidase (TPO) and CD64t expression on human thyroid epiCs^{TPO} (A) and epiCs^{TPO,CD64t} (B).

FIGS. 14A and 14B show representative flow cytometry histograms for the binding of free IgG1 F_{c} (alemtuzumab, which has no Fab binding site on thyroid epiCs) on human thyroid epiCs^{TPO} (FIG. 14A) and epiCs^{TPO,CD64t} (FIG. 14B). Only epiCs^{TPO,CD64t} were able to bind IgG1.

FIGS. 15A and 15B show thyroxine production by epiCs^{TPO} (FIG. 15A) and epiCs^{TPO,CD64t} (FIG. 15B) measured in an Elisa assay. There was no difference in thyroxine production in the presence or absence of 1 μg/ml anti-CD52 IgG1 antibodies (mean±SD, three independent replicates per group and time point).

FIG. 16. Human thyroid epithelial cells (epiCs) expressing thyroid peroxidase (TPO) in vitro underwent Antibody-mediated rejection (AMR) with increasing concentrations of an anti-TPO antibody via NK cell ADCC (upper row). Human thyroid epiCs additionally expressing CD64t were protected from ADCC across all antibody concentrations (lower row).

FIG. 17 shows human thyroid epiCs^{TPO} in impedance NK cell ADCC assays with different concentrations of an anti-TPO IgG1 antibody. Thyroid epiCs^{TPO} were killed in a concentration-dependent manner (mean±SD, three independent replicates per group and time point).

FIG. 18 shows human thyroid epiCs^{TPO} in impedance CDC assays with different concentrations of an anti-TPO IgG1 antibody. Thyroid epiCs^{TPO} were killed in a concentration-dependent manner (mean±SD, three independent replicates per group and time point).

FIG. 19 shows human thyroid epiCs^{TPO,CD64t} in impedance NK cell ADCC assays with different concentrations of an anti-TPO IgG1 antibody. Thyroid epiCs^{TPO,CD64t} were completely protected from killing (mean±SD, three independent replicates per group and time point).

FIG. 20 shows human thyroid epiCs^{TPO} in impedance CDC assays with different concentrations of an anti-TPO IgG1 antibody. Thyroid epiCs^{TPO,CD64t} were completely protected from killing (mean±SD, three independent replicates per group and time point).

FIGS. 21A, 21B, and 21C: Human thyroid epiCs^{TPO} and epiCs^{TPO,CD64t} were incubated with serum from Hashimoto's patients 1 (FIG. 21A), 2 (FIG. 21B), and 3 (FIG. 21C) in impedance CDC assays. These patients had anti-TPO antibody titers 21-fold (FIG. 21A), 26.3-fold (FIG. 21B), and 29.6-fold (FIG. 21C) over the upper level of normal (0.03 U/ml). Thyroid epiCs^{TPO} were expeditiously killed in these assays, but epiCs^{TPO,CD64t} survived and remained completely unaffected (mean±SD, three independent replicates per group and time point).

FIGS. 22A, 22B, and 22C: FIG. 22A shows the experimental setup of an in vivo antibody-killing assay. A total of 5×10⁴ human thyroid epiCs^{TPO} or epiCs^{TPO,CD64t} were injected subcutaneously into immunodeficient NSG mice (NOD Cg-Prkdc^{scid}l12rg^{mIwjl}/SzJ) with 10⁶ human NK cells. Both groups received 3 subcutaneous doses of anti-TPO 1 mg on days 0, 1, and 2. BLI signals of thyroid epiCs^{TPO} (FIG. 22B) and epiCs^{TPO,CD64t} (FIG. 22C) were followed and showed that thyroid epiCs^{TPO} grafts rapidly vanished, while all epiCs^{TPO,CD64t} survived (All single animals are plotted and the BLI images for one representative animal are shown per group).

FIGS. 23A and 23B show representative flow cytometry histograms for CD64t expression on human beta cells (FIG. 23A) or CD64t-expressing beta cells^{CD64t} (FIG. 23B). Beta cells^{CD64t} showed strong CD64t expression.

FIGS. 24A and 24B show flow cytometry histograms for the binding of free IgG1 F_{c} (alemtuzumab) on beta cells (FIG. 24A) and beta cells^{CD64t} (FIG. 24B). Only beta cells^{CD64t} showed marked IgG1 F_{c} binding in a concentration-dependent manner.

FIGS. 25A and 25B show glucose sensing and insulin production by human beta cells (FIG. 25A) and beta cells^{CD64t} (FIG. 25B) in ELISA assays. The assays were performed under low (2 mM) and high (20 mM) glucose conditions. Both beta cells and beta cells^{CD64t} produced significantly more insulin in high glucose condition. Glucose sensing and insulin production was not affected by the presence or absence of 1 μg/ml anti-CD52 IgG1 antibodies (mean±SD, three independent replicates per group and time point).

FIG. 26. Human islet cells (HLA-A2 positive) underwent in vitro antibody-mediated killing when challenged with an anti-HLA-A2 IgG antibody and NK effector cells (upper row). Islet cells transduced to express CD64t were protected from anti-HLA-A2 antibody-mediated killing across all antibody concentrations (lower row).

FIG. 27 shows human beta cells challenged in impedance CDC assays with different concentrations of an anti-HLA-A2 IgG1 antibody. The HLA-A2-expressing beta cells were killed in a concentration-dependent manner (mean±SD, three independent replicates per group and time point).

FIG. 28 shows human beta cells^{CD64t} challenged in impedance CDC assays with different concentrations of an anti-HLA-A2 IgG1 antibody. The HLA-A2-expressing beta cells^{CD64t} were completely resistant against CDC killing (mean±SD, three independent replicates per group and time point).

FIGS. 29A, 29B, 29C, and 29D: FIG. 29A shows the experimental setup of in vivo beta cell survival experiments. A total of 5×10⁴ human beta cells or beta cells^{CD64t} were injected subcutaneously into NSG mice with 10⁶ human NK cells. Both groups received 3 subcutaneous 1 mg doses of anti-HLA-A2 IgG1 on days 0, 1, and 2. BLI signals of beta cells (FIG. 29B) and beta cells^{CD64t} (FIG. 29C) were followed. All beta cell grafts were rejected within only 2 days. All beta cell^{CD64t} grafts remained unaffected and survived the study period. As controls, 5×10⁴ human beta cells were injected subcutaneously into NSG mice without NK cells and without antibodies (FIG. 29D). The survival kinetic of beta cell^{CD64t} grafts challenged with anti-HLA-A2 and NK cells was indistinguishable from that of beta cell grafts without antibodies and NK cells. That shows that beta cell^{CD64t} grafts were completely protected against antibody-mediated killing in vivo. All single animals are plotted and the BLI images for one representative animal are shown per group.

FIG. 30 shows representative flow cytometry histograms for anti-CD19 scFv and CD64t expression on human CAR-T (A) and CAR-T^{CD64t} (B). The anti-CD19 scFv antibody specifically recognizes the anti-CD19 CAR (mean±SD, three independent replicates per group and time point).

FIGS. 31A and 31B show representative flow cytometry histograms for the binding of free IgG1 F_{c} (anti-TPO IgG1) to CAR-T cells (FIG. 31A) and CAR-T^{CD64t} cells (FIG. 31B).

Only CAR-T^{CD64t} were able to bind IgG1 in a concentration-dependent manner (mean±SD, three independent replicates per group and time point).

FIG. 32 shows the kinetics of CD19⁺ NALM target cell killing by T cells, CAR-T cells, and CAR-T^{CD64}t. The killing speed is expressed as hours it takes for the cell index to drop from 1 to 0.5. Different T cell-to-NALM ratios are shown (mean±SD, three independent replicates per group and time point).

FIG. 33 shows the kinetics of CD19⁺ NALM target cell killing by CAR-T^{CD64t} in the presence and absence of 1 g/ml anti-CD52. The killing speed is expressed as hours it takes for the cell index to drop from 1 to 0.5. Different CAR-T cell-to-NALM ratios are shown (mean±SD, three independent replicates per group and time point).

FIG. 34 shows human CAR-T cells in impedance NK cell ADCC assays with antibodies against HLA (anti-HLA-A2), non-HLA (anti-CD52, anti-CD3), rhesus blood type antigen D (anti-Rh(D)), and against the CAR (anti-CD19 scFv) at 1 μg/ml (mean±SD, three independent replicates per group and time point). With all antibodies, there was very rapid killing of CAR-T cells.

FIG. 35 shows human CAR-T cells in impedance CDC assays with antibodies against HLA (anti-HLA-A2), non-HLA (anti-CD52, anti-CD3), rhesus blood type antigen D (anti-Rh(D)), and against the CAR (anti-CD19 scFv) at 1 μg/ml (mean±SD, three independent replicates per group and time point). Again, with all antibodies, there was very rapid killing of CAR-T cells.

FIG. 36 shows human CAR-T^{CD64t} cells in impedance NK cell ADCC assays with antibodies against HLA (anti-HLA-A2), non-HLA (anti-CD52, anti-CD3), rhesus blood type antigen D (anti-Rh(D)), and against the CAR (anti-CD19 scFv) at 1 μg/ml (mean±SD, three independent replicates per group and time point). The CAR-T^{CD64t} cells were completely resistant against ADCC killing.

FIG. 37 shows human CAR-T^{CD64t} cells in impedance CDC assays with antibodies against HLA (anti-HLA-A2), non-HLA (anti-CD52, anti-CD3), rhesus blood type antigen D (anti-Rh(D)), and against the CAR (anti-CD19 scFv) at 1 μg/ml (mean±SD, three independent replicates per group and time point). The CAR-T^{CD64t} Cells were completely resistant against CDC killing.

FIGS. 38A and 38B show representative flow cytometry histograms for CD64t expression on human NK cells (FIG. 38A) and NK^{CD64t} cells (FIG. 38B). Only NK^{CD64t} cells express CD64t.

FIGS. 39A and 39B show representative flow cytometry histograms for the binding of free IgG1 F_{c} (anti-TPO IgG1) to NK cells (FIG. 39A) and NK^{CD64t} cells (FIG. 39B). Only NK^{CD64t} cells were able to bind IgG1 in a concentration-dependent manner.

FIG. 40 shows human NK cells challenged in impedance NK cell ADCC assays with different concentrations of an anti-CD52 IgG1 antibody (alemtuzumab). The CD52-expressing NK cells were killed in a concentration-dependent manner (mean±SD, three independent replicates per group and time point).

FIG. 41 shows human NK cells challenged in impedance CDC assays with different concentrations of an anti-CD52 antibody. The CD52-expressing NK cells were killed in a concentration-dependent manner (mean±SD, three independent replicates per group and time point).

FIG. 42 shows human NK^{CD64t} cells challenged in impedance NK cell ADCC assays with different concentrations of an anti-CD52 IgG1 antibody (alemtuzumab). The CD52-expressing NK^{CD64t} cells were completely protected from ADCC killing (mean±SD, three independent replicates per group and time point).

FIG. 43 shows human NK^{CD64t} cells challenged in impedance CDC assays with different concentrations of an anti-CD52 IgG1 antibody (alemtuzumab). The CD52-expressing NK^{CD64t} cells were completely protected from CDC killing (mean±SD, three independent replicates per group and time point).

### V. DETAILED DESCRIPTION OF THE INVENTION

### A. Introduction

The invention provides, for the first time, cells that comprise truncated CD16, CD32, or CD64 expression to evade antibody-dependent cellular cytotoxicity (ADCC) or complement-dependent cytotoxicity (CDC) while eliminating intracellular signaling. The cells may be primary cells or pluripotent cells, including hypoimmune pluripotent cells (HIP) or ABO blood type O Rhesus Factor negative HIP cells (HIPO−), that further comprise the enhanced CD64 expression. The cells may also be pancreatic islet cells, thyroid cells, chimeric antigen receptor (CAR) cells, T cells, NK cells, ILCs, endothelial cells, dopaminergic neurons, cardiomyocytes, or retinal pigment endothelium cells used for treating diseases or rehabilitating damaged tissues.

Antibody-mediated rejection (AMR) after solid organ transplantation was first discovered as a distinct clinicopathological entity in 1997. Decades after the concept of cellular rejection had been widely accepted, and standardized nomenclature and diagnostic criteria were developed for kidney, heart, lung, liver, and pancreatic transplantation. A hallmark of AMR is the presence of graft-specific antibodies in combination with graft damage.

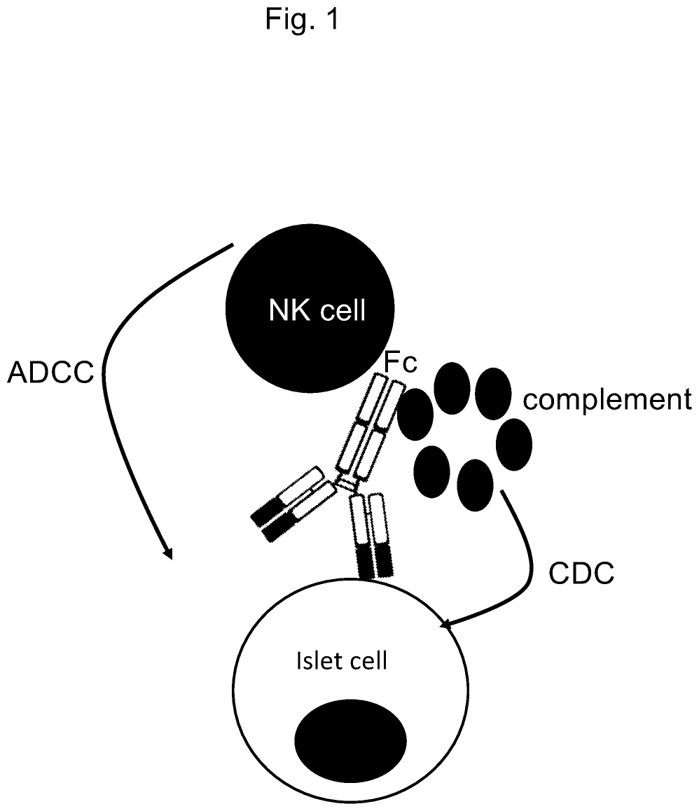

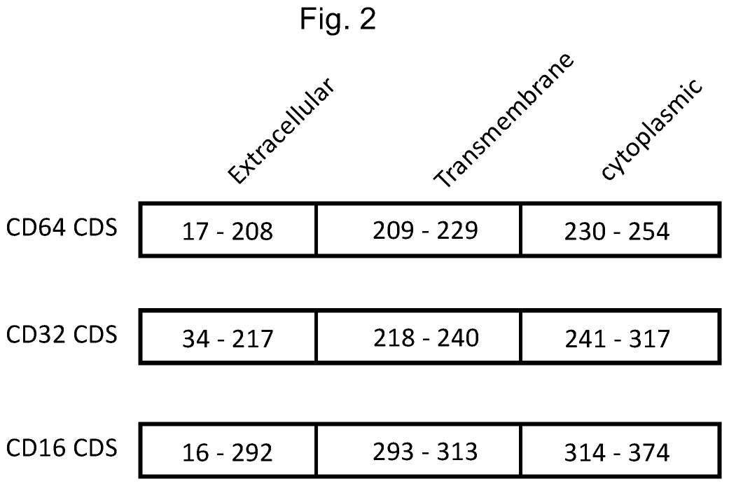

When an antibody binds to an HLA or non-HLA antigen on a cell via its Fab regions, the F_{c} is bound by NK cells (mostly via their CD16 receptor) and mediate antibody-mediated cellular cytotoxicity (ADCC) or AMR in a transplant setting. Complement can also bind to F_{c} and activate its cascade and form the membrane attack complex (MAC) for CDC killing (FIG. 1).

There are three layers of antigens to which antibodies can be directed. First and most relevant in the transplant setting are human leukocyte antigens (HLA), the most polymorphic histocompatibility complexes crucial for T cell activation. Patients with antibodies to their grafts were consistently shown to have inferior graft survival across organ systems.

Second are the highly polymorphic non-HLA antigens such as the MHC Class I-Related Sequence A (MICA), a cell surface glycol-protein encoded within the human HLA genes. It does not associate with β2-microglobulin nor present peptides. Anti-MICA antibodies have been associated with accelerated graft failure in solid organ transplantation.

MICA polymorphism and expression on pancreatic islet cells is associated with type 1 diabetes mellitus (T1DM).

Third, antibodies against cell type-specific surface antigens generated after solid organ transplantation mediate AMR.

These AMR reactions occur in transplant recipients despite the use of effective systemic immunosuppression. In some autoimmune diseases, autoantibodies cause or enhance the destruction of the target cells and persist as part of the disease. Patients with autoimmune thyroiditis or type 1 diabetes mellitus (T1DM) have a high prevalence of anti-thyroid epithelial cell or anti-β cell antibodies, respectively, which may persist for many years.

Patients with cellular grafts using long-term benign regenerative approaches in immunocompetent patients may eventually experience some form of antibody-mediated immune attack, dependent on the cell source and disease treated. Thyroid cells and islet cells express all three layers of antigens and are most vulnerable against all forms of AMR. For this reason, these cell types exemplify the antibody evasive technology disclosed herein.

Other cell types are implicated and include allogeneic human embryonic stem cell-derived cardiac progenitors and mesenchymal stroma cell derivatives.

The invention provides a gene editing strategy that effectively protects transplanted cells such as islet cells and thyroid cells from AMR. It establishes protection from antibodies against HLA-, non-HLA-, and cell type-specific antigens. In some embodiments it is combined with additional hypoimmune edits to silence cellular rejection.

The invention provides antibody resistance that builds upon the HIP concept and utilizes non-immunogenic components. The systemic use of microbial IgG-degrading enzymes has successfully been used to deplete total IgG and HLA antibodies in highly sensitized patients before their kidney transplantation(Jordan, S. C. et al., N Engl J Med 377, 442-453 (2017)). More recently, this endopeptidase was shown to cleave IgG bound to target cells (Peraro, L. et al., Mol Ther (2021)). Pre-existing antibodies against these bacterial enzymes, however, are widely prevalent in the healthy population (Akesson, P. et al., J Infect Dis 189, 797-804 (2004)). They spike during streptococcal infections (Lei, B. et al., Nat Med 7, 1298-1305 (2001); Okamoto, S., et al. Vaccine 23, 4852-4859 (2005)), and might themselves add unwanted immunogenicity. Human CD64 and its truncated form CD64t are non-immunogenic, have high affinity for IgG(Bruhns, P. et al., Blood 113, 3716-3725 (2009)), and are very effective against antibody killing in several translationally relevant cell types.

The invention applies this technology to regenerative cell therapeutics, especially for diseases with an underlying autoimmune component in which antibodies are present that would destroy the transplanted cells. Regenerative cell therapeutics would be destroyed similarly to the native cells if autoimmunity is not circumvented (Hollenberg, A. N. et al., Mol Cell Endocrinol 445, 35-41 (2017)).

The invention shows that engineered epithelial cells (epC) expressing thyroid peroxidase (TPO) and CD64 (epiCs^{TPO,CD64t}) were protected against clinically relevant anti-TPO killing. Current stem cell-derived pancreatic islet cells in clinical trials for patients with Type-I Diabetes Melitis are currently produced from embryonic stem cells (Melton, D. The promise of stem cell-derived islet replacement therapy. Diabetologia 64, 1030-1036 (2021)). They are transplanted across an HLA mismatch. Despite the use of encapsulation strategies to protect the islet cells from the host immune system, however, antibodies against microencapsulated graft cells have been observed. (Desai, T. & Shea, L. D., Nat Rev Drug Discov 16, 338-350 (2017); Duvivier-Kali et al., Am J Transplant 4, 1991-2000 (2004)). Forced overexpression of CD64t on human beta cells was sufficient to protect them from anti-HLA ADCC and CDC.

Most success in CAR-T cell therapy has been achieved with B cell neoplasms in which patients are treated with lymphocytotoxic drugs and the CAR-T cells directly target the source for antibody production (Brudno, J. N. & Kochenderfer, J. N., Nat Rev C/in Oncol 15, 31-46 (2018)). Antibody induction, however, is still regularly observed (Till, B. G. et al. Blood 112, 2261-2271 (2008)). An antibody response against the CAR-T cells has been shown to prevent expansion of CAR-T cells upon re-infusion (Peraro, L. et al. Incorporation of bacterial immunoevasins to protect cell therapies from host antibody-mediated immune rejection. Mol Ther (2021)) and hamper persistence. The expression of CD64t on CAR-T cells makes them resistant to anti-CAR-T cell antibodies without affecting their specific killing capacity. Since early immune clearance is even more common in non-B cell cancer, CAR-T^{CD64t} may be more efficient for these indications. The F_{c} sequestration mechanism reliably builds protection against antibodies in several cell types and further advances the immune evasion concept for allogeneic regenerative and immune-oncology cell therapeutics. (See WO2021076427, incorporated by reference herein in its entirety.)

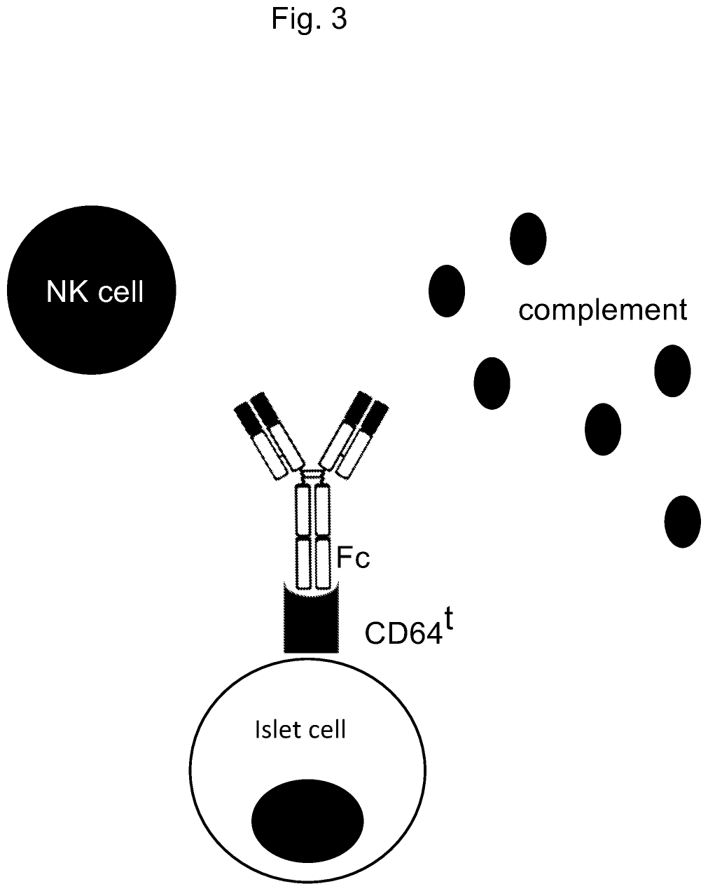

CD64 is the high-affinity human IgG receptor FcγRI capable of capturing free serum IgG. In order to achieve resistance against AMR, the Fc binding capacity of CD64 was utilized. To avoid alterations of the engineered cells from unwanted intracellular signaling, however, the invention provides a truncated form of CD64 (CD64t) on cells that is incapable of inducing intracellular signaling. The intracellular tail was cut off in the CD64 coding sequence (CDS) before it was cloned into a lentivirus for expression in pluripotent cells. The CD64t captures free IgG Fc without inducing downstream signaling (FIG. 3).

One specific indication for this technology is the cellular treatment of autoimmune diseases. Antibodies against autologous epitopes cause beta cell death in type 1 diabetes and thyroid cells in autoimmune thyroiditis. Antibodies may bind to HLA class II molecules. Additionally, HLA-independent antibodies have been described. Should a cell that is used for regenerative therapy have the same epitope, it will also be bound by these antibodies and be killed. The CD64 sequestration of the invention prevents ADCC and CDC.

In some embodiments of the invention, HypoImmunogenic Pluripotent (“HIP”) cells are modified to express CD16t, CD32t, or CD64t (HIP/CD16t, HIP/CD32t, or HIP/CD64t cells). HIP cells avoid host immune responses due to several genetic manipulations as outlined herein. The cells lack major immune antigens that trigger immune responses and are engineered to avoid phagocytosis and NK cell killing. In some embodiments, the HIP cells are made by eliminating the activity of both alleles of a B2M gene in an induced pluripotent stem cell (iPSC); eliminating the activity of both alleles of a CIITA gene in the iPSC; and increasing the expression of CD47 in the iPSC. HIP cells are described in detail in WO2018132783, incorporated by reference herein in its entirety. In other embodiments, HIP cells express a SIRPα-engager molecule as disclosed in PCT/US2021/062008, incorporated by reference herein in its entirety.

In other embodiments of the invention, HypoImmunogenic Pluripotent Blood group O Rh-(“HIPO−”) cells are modified to express CD16t, CD32t, or CD64t (HIPO−/CD16t cells, HIPO−/CD32t, or HIPO−/CD64t cells). HIPO− cells avoid host immune responses due to several genetic or enzymatic manipulations as outlined herein. The cells lack major blood group and immune antigens that trigger immune responses and are engineered to avoid rejection, phagocytosis, or killing. This allows the derivation of “off-the-shelf” cell products for generating specific tissues and organs. The benefit of being able to use human allogeneic HIPO− cells and their derivatives in human patients provides significant benefits, including the ability to avoid long-term adjunct immunosuppressive therapy and drug use generally seen in allogeneic transplantations. They also provide significant cost savings as cell therapies can be used without requiring individual treatments for each patient. HIPO−/CD16, HIPO−/CD32, or HIPO−/CD64 cells may serve as a universal cell source for the generation of universally-acceptable derivatives. HIPO− cells are described in detail in U.S. Prov. Appl. No. 62/846,399 and U.S. Pat. No. 62,855,499 each of which are incorporated by reference herein in their entirety.

### B. Definitions

The term “pluripotent cells” refers to cells that can self-renew and proliferate while remaining in an undifferentiated state and that can, under the proper conditions, be induced to differentiate into specialized cell types. The term “pluripotent cells,” as used herein, encompass embryonic stem cells and other types of stem cells, including fetal, amnionic, or somatic stem cells. Exemplary human stem cell lines include the H9 human embryonic stem cell line. Additional exemplary stem cell lines include those made available through the National Institutes of Health Human Embryonic Stem Cell Registry and the Howard Hughes Medical Institute HUES collection (as described in Cowan, C. A. et. al, New England J Med. 350:13. (2004), incorporated by reference herein in its entirety.)

“Pluripotent stem cells” as used herein have the potential to differentiate into any of the three germ layers: endoderm (e.g. the stomach linking, gastrointestinal tract, lungs, etc), mesoderm (e.g. muscle, bone, blood, urogenital tissue, etc) or ectoderm (e.g. epidermal tissues and nervous system tissues). The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells, and “miPSCs” are murine induced pluripotent stem cells.

“Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least several, and in some embodiments, all of the markers from the following non-limiting list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics. As described herein, cells do not need to pass through pluripotency to be reprogrammed into endodermal progenitor cells and/or hepatocytes.

As used herein, “multipotent” or “multipotent cell” refers to a cell type that can give rise to a limited number of other particular cell types. For example, induced multipotent cells are capable of forming endodermal cells. Additionally, multipotent blood stem cells can differentiate itself into several types of blood cells, including lymphocytes, monocytes, neutrophils, etc.

As used herein, the term “oligopotent” refers to the ability of an adult stem cell to differentiate into only a few different cell types. For example, lymphoid or myeloid stem cells are capable of forming cells of either the lymphoid or myeloid lineages, respectively.

As used herein, the term “unipotent” means the ability of a cell to form a single cell type. For example, spermatogonial stem cells are only capable of forming sperm cells.

As used herein, the term “totipotent” means the ability of a cell to form an entire organism. For example, in mammals, only the zygote and the first cleavage stage blastomeres are totipotent.

As used herein, “non-pluripotent cells” refer to mammalian cells that are not pluripotent cells. Examples of such cells include differentiated cells as well as progenitor cells. Examples of differentiated cells include, but are not limited to, cells from a tissue selected from bone marrow, skin, skeletal muscle, fat tissue and peripheral blood. Exemplary cell types include, but are not limited to, fibroblasts, hepatocytes, myoblasts, neurons, osteoblasts, osteoclasts, and T-cells. The starting cells employed for generating the induced multipotent cells, the endodermal progenitor cells, and the hepatocytes can be non-pluripotent cells.

Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

A “somatic cell” is a cell forming the body of an organism. Somatic cells include cells making up organs, skin, blood, bones and connective tissue in an organism, but not germ cells.

Cells can be from, for example, human or non-human mammals. Exemplary non-human mammals include, but are not limited to, mice, rats, cats, dogs, rabbits, guinea pigs, hamsters, sheep, pigs, horses, bovines, and non-human primates. In some embodiments, a cell is from an adult human or non-human mammal. In some embodiments, a cell is from a neonatal human, an adult human, or non-human mammal.

As used herein, the terms “subject” or “patient” refers to any animal, such as a domesticated animal, a zoo animal, or a human. The “subject” or “patient” can be a mammal like a dog, cat, bird, livestock, or a human. Specific examples of “subjects” and “patients” include, but are not limited to, individuals (particularly human) with a disease or disorder related to the liver, heart, lung, kidney, pancreas, brain, neural tissue, blood, bone, bone marrow, and the like.

Mammalian cells can be from humans or non-human mammals. Exemplary non-human mammals include, but are not limited to, mice, rats, cats, dogs, rabbits, guinea pigs, hamsters, sheep, pigs, horses, bovines, and non-human primates (e.g., chimpanzees, macaques, and apes).

By “hypo-immunogenic pluripotent” cell or “HIP” cell herein is meant a pluripotent cell that retains its pluripotent characteristics and yet gives rise to a reduced immunological rejection response when transferred into an allogeneic host. In preferred embodiments, HIP cells do not give rise to an immune response. Thus, “hypo-immunogenic” refers to a significantly reduced or eliminated immune response when compared to the immune response of a parental (i.e. “wt”) cell prior to immunoengineering as outlined herein. In many cases, the HIP cells are immunologically silent and yet retain pluripotent capabilities. Assays for HIP characteristics are outlined below.

By “HIP/CD16t”, “HIP/CD32t”, or “HIP/CD64t” cell herein is meant a HIP cell that has a truncated CD16, CD32, or CD64 protein, respectively, on the cell surface. The truncation removes the intracellular signaling domain. In an alternative embodiment, the signaling domain is not truncated, but rather, modified to eliminate a signaling function. Such modifications may be amino acid substitutions or internal deletions.

By “hypo-immunogenic pluripotent cell O−” “hypo-immunogenic pluripotent ORh−” cell or “HIPO−” cell herein is meant a HIPO− cell that is also ABO blood group O and Rhesus Factor Rh−. HIPO− cells may have been generated from O− cells, enzymatically modified to be O−, or genetically engineered to be O−.

By “HIPO−/CD16t”, “HIPO−/CD32t”, or “HIPO−/CD64t” cell herein is meant a HIPO− cell that has a truncated CD16, CD32, or CD64 protein, respectively, on the cell surface. The truncation removes the intracellular signaling domain. In an alternative embodiment, the signaling domain is not truncated, but rather, mutated to eliminate a signaling function.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

By “gene knock out” herein is meant a process that renders a particular gene inactive in the host cell in which it resides, resulting either in no protein of interest being produced or an inactive form. As will be appreciated by those in the art and further described below, this can be accomplished in a number of different ways, including removing nucleic acid sequences from a gene, or interrupting the sequence with other sequences, altering the reading frame, or altering the regulatory components of the nucleic acid. For example, all or part of a coding region of the gene of interest can be removed or replaced with “nonsense” sequences, all or part of a regulatory sequence such as a promoter can be removed or replaced, translation initiation sequences can be removed or replaced, etc.

By “gene knock in” herein is meant a process that adds a genetic function to a host cell. This causes increased levels of the encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

“β-2 microglobulin” or “02M” or “B2M” protein refers to the human 02M protein that has the amino acid and nucleic acid sequences shown below; the human gene has accession number NC_000015.10:44711487-44718159.

“CD47 protein” protein refers to the human CD47 protein that has the amino acid and nucleic acid sequences shown below; the human gene has accession number NC_000016.10:10866208-10941562. CD47 is a ligand for SIRPα (a “SIRPα engager” molecule). CD47 is a “marker-of-self” protein that can be overexpressed broadly across tumor types. It is emerging as a novel potent macrophage immune checkpoint for cancer immunotherapy. CD47 in tumor cells sends a “don't-eat-me” signal that inhibits macrophage phagocytosis.

“CIITA protein” protein refers to the human CIITA protein that has the amino acid and nucleic acid sequences shown below; the human gene has accession number NC_000003.12:108043094-108094200.

By “wild type” in the context of a cell means a cell found in nature. However, in the context of a pluripotent stem cell, as used herein, it also means an iPSC that may contain nucleic acid changes resulting in pluripotency but did not undergo the gene editing procedures of the invention to achieve hypo-immunogenicity.

By “syngeneic” herein refers to the genetic similarity or identity of a host organism and a cellular transplant where there is immunological compatibility; e.g. no immune response is generated.

By “allogeneic” herein refers to the genetic dissimilarity of a host organism and a cellular transplant where an immune response is generated.

By “B2M−/−” herein is meant that a diploid cell has had the B2M gene inactivated in both chromosomes. As described herein, this can be done in a variety of ways.

By “CIITA −/−” herein is meant that a diploid cell has had the CIITA gene inactivated in both chromosomes. As described herein, this can be done in a variety of ways.

By “CD47 tg” (standing for “transgene”) or “CD47+”) herein is meant that the host cell expresses CD47, in some cases by having at least one additional copy of the CD47 gene.

An “Oct polypeptide” refers to any of the naturally-occurring members of Octamer family of transcription factors, or variants thereof that maintain transcription factor activity, similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Oct polypeptides include Oct-1, Oct-2, Oct-3/4, Oct-6, Oct-7, Oct-8, Oct-9, and Oct-11. Oct3/4 (referred to herein as “Oct4”) contains the POU domain, a 150 amino acid sequence conserved among Pit-1, Oct-1, Oct-2, and uric-86. (See, Ryan, A. K. & Rosenfeld, M. G., Genes Dev. 11:1207-1225 (1997), incorporated herein by reference in its entirety.) In some embodiments, variants have at least 85%, 90%, or 95% amino acid sequence identity across their whole sequence compared to a naturally occurring Oct polypeptide family member such as to those listed above or such as listed in Genbank accession number NP-002692.2 (human Oct4) or NP-038661.1 (mouse Oct4). Oct polypeptides (e.g., Oct3/4 or Oct 4) can be from human, mouse, rat, bovine, porcine, or other animals. Generally, the same species of protein will be used with the species of cells being manipulated. The Oct polypeptide(s) can be a pluripotency factor that can help induce multipotency in non-pluripotent cells.

A “Klf polypeptide” refers to any of the naturally-occurring members of the family of Krnppel-like factors (Klfs), zinc-finger proteins that contain amino acid sequences similar to those of the Drosophila embryonic pattern regulator Kruppel, or variants of the naturally-occurring members that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. (See, Dang, D. T., Pevsner, J. & Yang, V. W., Cell Biol. 32:1103-1121 (2000), incorporated by reference herein in its entirety.) Exemplary Klf family members include, Klf1, Klf2, Klf3, Klf-4, Klf5, Klf6, Klf7, Klf8, Klf9, Kifl0, Klfl1, Klfl2, Klfl3, Klfl4, Klfl5, Klfl6, and Klfl7. Klf2 and Klf-4 were found to be factors capable of generating iPS cells in mice, and related genes Klf1 and Klf5 did as well, although with reduced efficiency. (See, Nakagawa, et al., Nature Biotechnology 26:101-10⁶ (2007), incorporated by reference herein in its entirety.) In some embodiments, variants have at least 85%, 90%, or 95% amino acid sequence identity across their whole sequence compared to a naturally occurring Klf polypeptide family member such as to those listed above or such as listed in Genbank accession number CAX16088 (mouse Klf4) or CAX14962 (human Klf4). Klf polypeptides (e.g., Klf1, Klf4, and Klf5) can be from human, mouse, rat, bovine, porcine, or other animals. Generally, the same species of protein will be used with the species of cells being manipulated. The Klf polypeptide(s) can be a pluripotency factor. The expression of the Klf4 gene or polypeptide can help induce multipotency in a starting cell or a population of starting cells.

A “Myc polypeptide” refers to any of the naturally-occurring members of the Myc family. (See, e.g., Adhikary, S. & Eilers, M., Nat. Rev. Mol. Cell Biol. 6:635-645 (2005), incorporated by reference herein in its entirety.) It also includes variants that maintain similar transcription factor activity when compared to the closest related naturally occurring family member (i.e. within at least 50%, 80%, or 90% activity). It further includes polypeptides comprising at least the DNA-binding domain of a naturally occurring family member, and can further comprise a transcriptional activation domain. Exemplary Myc polypeptides include, e.g., c-Myc, N-Myc and L-Myc. In some embodiments, variants have at least 85%, 90%, or 95% amino acid sequence identity across their whole sequence compared to a naturally occurring Myc polypeptide family member, such as to those listed above or such as listed in Genbank accession number CAA25015 (human Myc). Myc polypeptides (e.g., c-Myc) can be from human, mouse, rat, bovine, porcine, or other animals. Generally, the same species of protein will be used with the species of cells being manipulated. The Myc polypeptide(s) can be a pluripotency factor.

A “Sox polypeptide” refers to any of the naturally-occurring members of the SRY-related HMG-box (Sox) transcription factors, characterized by the presence of the high-mobility group (HMG) domain, or variants thereof that maintain similar transcription factor activity when compared to the closest related naturally occurring family member (i.e. within at least 50%, 80%, or 90% activity). It also includes polypeptides comprising at least the DNA-binding domain of the naturally occurring family member, and can further comprise a transcriptional activation domain. (See, e.g., Dang, D. T. et al., Int. J. Biochem. Cell Biol. 32:1103-1121 (2000), incorporated by reference herein in its entirety.) Exemplary Sox polypeptides include, e.g., Sox1, Sox-2, Sox3, Sox4, Sox5, Sox6, Sox7, Sox8, Sox9, Sox10, Sox11, Sox12, Sox13, Sox14, Sox15, Sox17, Sox18, Sox-21, and Sox30. Sox1 has been shown to yield iPS cells with a similar efficiency as Sox2, and genes Sox3, Sox15, and Sox18 have also been shown to generate iPS cells, although with somewhat less efficiency than Sox2. (See, Nakagawa, et al., Nature Biotechnology 26:101-10⁶ (2007), incorporated by reference herein in its entirety.) In some embodiments, variants have at least 85%, 90%, or 95% amino acid sequence identity across their whole sequence compared to a naturally occurring Sox polypeptide family member such as to those listed above or such as listed in Genbank accession number CAA83435 (human Sox2). Sox polypeptides (e.g., Sox1, Sox2, Sox3, Sox15, or Sox18) can be from human, mouse, rat, bovine, porcine, or other animals. Generally, the same species of protein will be used with the species of cells being manipulated. The Sox polypeptide(s) can be a pluripotency factor. As discussed herein, SOX2 proteins find particular use in the generation of iPSCs.

By “differentiated hypo-immunogenic pluripotent cells” or “differentiated HIP cells” or “dHIP cells” herein is meant iPS cells that have been engineered to possess hypoimmunogenicity (e.g. by the knock out of B2M and CIITA and the knock in of CD47) and then are differentiated into a cell type for ultimate transplantation into subjects. Thus, for example HIP cells can be differentiated into hepatocytes (“dHIP hepatocytes”), into beta-like pancreatic cells or islet organoids (“dHIP beta cells”), into endothelial cells (“dHIP endothelial cells”), etc. Parallel definitions apply to “differentiated HIP/CD64t” and differentiated HIPO−/CD64t cells.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

“Inhibitors,” “activators,” and “modulators” affect a function or expression of a biologically-relevant molecule. The term “modulator” includes both inhibitors and activators. They may be identified using in vitro and in vivo assays for expression or activity of a target molecule.

“Inhibitors” are agents that, e.g., inhibit expression or bind to target molecules or proteins. They may partially or totally block stimulation or have protease inhibitor activity. They may reduce, decrease, prevent, or delay activation, including inactivation, desensitizion, or down regulation of the activity of the described target protein. Modulators may be antagonists of the target molecule or protein.

“Activators” are agents that, e.g., induce or activate the function or expression of a target molecule or protein. They may bind to, stimulate, increase, open, activate, or facilitate the target molecule activity. Activators may be agonists of the target molecule or protein.

“Homologs” are bioactive molecules that are similar to a reference molecule at the nucleotide sequence, peptide sequence, functional, or structural level. Homologs may include sequence derivatives that share a certain percent identity with the reference sequence.

Thus, in one embodiment, homologous or derivative sequences share at least a 70 percent sequence identity. In a specific embodiment, homologous or derivative sequences share at least an 80 or 85 percent sequence identity. In a specific embodiment, homologous or derivative sequences share at least a 90 percent sequence identity. In a specific embodiment, homologous or derivative sequences share at least a 95 percent sequence identity. In a more specific embodiment, homologous or derivative sequences share at least an 50, 55, 60, 65, 70, 75, 85, 86, 87, 88, 89, 90, 91, 92, 93, 94, 95, 96, 97, 98, or 99 percent sequence identity.

Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting, generating, and screening for structural and functional homologs as well as derivatives are known in the art.

“Hybridization” generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al, Current Protocols in Molecular Biology, Wiley Interscience Publishers (1995), incorporated by reference herein in its entirety.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures.

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 Mm sodium phosphate buffer at Ph 6.5 with 750 Mm sodium chloride, 75 Mm sodium citrate at 42° C.; or (3) overnight hybridization in a solution that employs 50% formamide, 5×SSC (0.75 M NaCl, 0.075 M sodium citrate), 50 Mm sodium phosphate (Ph 6.8), 0.1% sodium pyrophosphate, 5×Denhardt's solution, sonicated salmon sperm DNA (50 μl/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein the term “modification” refers to an alteration that physically differentiates the modified molecule from the parent molecule. In one embodiment, an amino acid change in a CD16, CD32, CD64, CD47, HSVtk, EC-CD, or iCasp9 variant polypeptide prepared according to the methods described herein differentiates it from the corresponding parent gene or cell that has not been modified according to the methods described herein, such as wild-type proteins, a naturally occurring mutant proteins or another engineered protein that does not include the modifications of such variant polypeptide. In another embodiment, a variant polypeptide includes one or more modifications that differentiates the function of the variant polypeptide from the unmodified polypeptide. For example, an amino acid change in a variant polypeptide affects its receptor binding profile. In other embodiments, a variant polypeptide comprises substitution, deletion, or insertion modifications, or combinations thereof. In another embodiment, a variant polypeptide includes one or more modifications that increases its affinity for a receptor compared to the affinity of the unmodified polypeptide.

In one embodiment, a variant polypeptide includes one or more substitutions, insertions, or deletions relative to a corresponding native or parent sequence. In certain embodiments, a variant polypeptide includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31-40, 41 to 50, or 51 or more modifications.

By “episomal vector” herein is meant a genetic vector that can exist and replicate autonomously in the cytoplasm of a cell; e.g. it is not integrated into the genomic DNA of the host cell. A number of episomal vectors are known in the art and described below.

By “knock out” in the context of a gene means that the host cell harboring the knock out does not produce a functional protein product of the gene. As outlined herein, a knock out can result in a variety of ways, from removing all or part of the coding sequence, introducing frameshift mutations such that a functional protein is not produced (either truncated or nonsense sequence), removing or altering a regulatory component (e.g. a promoter) such that the gene is not transcribed, preventing translation through binding to mRNA, etc. Generally, the knock out is effected at the genomic DNA level, such that the cells' offspring also carry the knock out permanently.

By “knock in” in the context of a gene means that the host cell harboring the knock in has more functional protein active in the cell. As outlined herein, a knock in can be done in a variety of ways, usually by the introduction of at least one copy of a transgene (tg) encoding the protein into the cell, although this can also be done by replacing regulatory components as well, for example by adding a constitutive promoter to the endogeneous gene.

In general, knock in technologies result in the integration of the extra copy of the transgene into the host cell.

### VI. Cells of the Invention

The invention provides compositions and methodologies for generating pluripotent cells that express CD16t, CD32t, or CD64t. In some aspects of the invention, the cells will be derived from induced pluripotent stem cells (IPSC), O− induced pluripotent stem cells (iPSCO−), embryonic stem cells (ESC), O− embryonic stem cells (ESCO−), hypoimmunogenic pluripotent (HIP) cells, hypoimmunogenic pluripotent O− (HIPO−) cells, or cells derived or differentiated therefrom. In other aspects, the cells are primary cells, including gene-edited primary cells. The cells of the invention may also be pancreatic islet cells, thyroid cells, chimeric antigen receptor (CAR) cells, endothelial cells, dopaminergic neurons, cardiomyocytes, or retinal pigment endothelium cells used for treating diseases or rehabilitating damaged tissues.

### A. Methodologies for Genetic Alterations

The invention includes methods of modifying nucleic acid sequences within cells or in cell-free conditions to generate both cells with CD16t, CD32t, or CD64t expression. Exemplary technologies include homologous recombination, knock-in, ZFNs (zinc finger nucleases), TALENs (transcription activator-like effector nucleases), CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

As will be appreciated by those in the art, a number of different techniques can be used to engineer the pluripotent cells of the invention, as well as the engineering of the iPSCs to become hypo-immunogenic as outlined herein.

In general, these techniques can be used individually or in combination. For example, these techniques can be used to generate F_{c} receptor-expressing cells where the receptor is truncated to remove some or all of the cytoplasmic signalling domain.

Alternatively, this domain may be modified by deletion, substitutions, or nonsense mutations.

Upon Fc binding, the cytoplasmic tail of CD64 interacts with the src-related family of tyrosine kinases (such as Fyn and Lyn) and Syk family kinases. They phosphorylate a cytoplasmic amino acid motif termed the immunoreceptor tyrosine-based activation motif (ITAM) on the associated FcR gamma chain (coded by its own gene FCER1G). This mediates the activation of immune cells.

Thus, the invention provides modifying the cytoplasmic tail that interact with the src-related family of tyrosine kinases and the Syk family kinases to prevent the activation of the FcR gamma chain ITAMs and thus prevent downstream signaling.

The CD64 cytoplasmic tail is the following 60 amino acids from SEQ ID NO:7:
RKELKRKKKWDLEISLDSGHEKKVISSLQEDRHLEEELKCQEQKEEQLQEGV HRKEPQGAT

In some embodiments, the cytoplasmic domain of the Fc receptors are exchanged for another cytoplasic domain with a different physiology. For instance, a cytoplasmic domain of a transferrin receptor (e.g. TfR1 or TfR2) promotes continuous endocytosis leading to a high receptor turnover. Thus, in certain embodiments, the Fc receptors are a chimera between an Fc receptor extracellular domain and a transferrin receptor cytoplasmic domain. This will promote endocytosis and disintegration of an IgG antibody that was initially bound to the Fc receptor domain on the cell surface. Thus, in preferred embodiments, the cells of the invention express a chimera comprising a CD16, CD32, or CD64 cell surface domain and a TfR1 or TfR2 cytoplasmic domain.

Moreover, these techniques may be used in the generation of the HIP cells. CRISPR may be used to reduce the expression of active B2M and/or CIITA protein in the engineered cells, with viral techniques (e.g. lentivirus) to knock in expression of CD47 or other SIRPα-Engager proteins. Also, as will be appreciated by those in the art, although one embodiment sequentially utilizes a CRISPR step to knock out B2M, followed by a CRISPR step to knock out CIITA with a final step of a lentivirus to knock in expression of CD47 or other SIRPα-Engager proteins, these genes can be manipulated in different orders using different technologies.

As is discussed more fully below, transient expression of reprogramming genes is generally done to generate/induce pluripotent stem cells.

a. CRISPR Technologies

In one embodiment, the cells are manipulated using clustered regularly interspaced short palindromic repeats)/Cas (“CRISPR”) technologies as is known in the art. CRISPR can be used to generate the starting iPSCs or to generate the HIP cells from the iPSCs. There are a large number of techniques based on CRISPR, see for example Doudna and Charpentier, Science doi:10.1126/science.1258096, hereby incorporated by reference. CRISPR techniques and kits are sold commercially.

b. TALEN Technologies

In some embodiments, the HIP cells of the invention are made using Transcription Activator-Like Effector Nucleases (TALEN) methodologies. TALEN are restriction enzymes combined with a nuclease that can be engineered to bind to and cut practically any desired DNA sequence. TALEN kits are sold commercially.

c. Zinc Finger Technologies

In one embodiment, the cells are manipulated using Zn finger nuclease technologies. Zn finger nucleases are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms, similar to CRISPR and TALENs.

d. Viral Based Technologies

There are a wide variety of viral techniques that can be used to generate the HIP cells of the invention (as well as for the original generation of the iPCSs), including, but not limited to, the use of retroviral vectors, lentiviral vectors, adenovirus vectors and Sendai viral vectors. Episomal vectors used in the generation of iPSCs are described below.

e. Down regulation of genes using interfering RNA

In other embodiments, genes that encode proteins used in HLA molecules are downregulated by RNAi technologies. RNA interference (RNAi) is a process where RNA molecules inhibit gene expression often by causing specific mRNA molecules to degrade. Two types of RNA molecules—microRNA (miRNA) and small interfering RNA (siRNA)—are central to RNA interference. They bind to the target mRNA molecules and either increase or decrease their activity. RNAi helps cells defend against parasitic nucleic acids such as those from viruses and transposons. RNAi also influences development.

sdRNA molecules are a class of asymmetric siRNAs comprising a guide (antisense) strand of 19-21 bases. They contain a 5′ phosphate, 2′Ome or 2′F modified pyrimidines, and six phosphotioates at the 3′ positions. They also contain a sense strand containing 3′ conjugated sterol moieties, 2 phospotioates at the 3′ position, and 2′Ome modified pyrimidines. Both strands contain 2′ Ome purines with continuous stretches of unmodified purines not exceeding a length of 3. sdRNA is disclosed in U.S. Pat. No. 8,796,443, incorporated herein by reference in its entirety.

For all of these technologies, well known recombinant techniques are used, to generate recombinant nucleic acids as outlined herein. In certain embodiments, the recombinant nucleic acids (either than encode a desired polypeptide, e.g. CD47, or disruption sequences) may be operably linked to one or more regulatory nucleotide sequences in an expression construct. Regulatory nucleotide sequences will generally be appropriate for the host cell and subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, or the expression of any other protein encoded by the vector, such as antibiotic markers.

Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s).

In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication. Fiers et al., Nature 273: 113-120 (1978). The immediate early promoter of the human cytomegalovirus is conveniently obtained as a HindIII E restriction fragment. Greenaway, P. J. et al., Gene 18: 355-360 (1982). The foregoing references are incorporated by reference in their entirety.

### B. Generation of Pluripotent Cells

The invention provides methods of producing non-immunogenic pluripotent cells from pluripotent cells. Thus, the first step is to provide the pluripotent stem cells.

The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al., World J Stem Cells 7(1):116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more “reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogeneous genes. This loss of the episomal vector(s) results in cells that are called “zero footprint” cells. This is desirable as the fewer genetic modifications (particularly in the genome of the host cell), the better. Thus, it is preferred that the resulting hiPSCs have no permanent genetic modifications.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g. fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen.

In general, these reprogramming factor genes are provided on episomal vectors such as are known in the art and commercially available. For example, ThermoFisher/Invitrogen sell a sendai virus reprogramming kit for zero footprint generation of hiPSCs, see catalog number A34546. ThermoFisher also sells EBNA-based systems as well, see catalog number A14703.

In addition, there are a number of commercially available hiPSC lines available; see, e.g., the Gibco® Episomal hiPSC line, K18945, which is a zero footprint, viral-integration-free human iPSC cell line (see also Burridge et al, 2011, supra).

In general, as is known in the art, iPSCs are made from non-pluripotent cells such as CD34+ cord blood cells, fibroblasts, etc., by transiently expressing the reprogramming factors as described herein.

For example, successful iPSCs were also generated using only Oct3/4, Sox2 and Klf4, while omitting the C-Myc, although with reduced reprogramming efficiency.

In general, iPSCs are characterized by the expression of certain factors that include KLF4, Nanog, OCT4, SOX2, ESRRB, TBX3, c-Myc and TCL1. New or increased expression of these factors for purposes of the invention may be via induction or modulation of an endogenous locus or from expression from a transgene.

For example, murine iPSCs can be generated using the methods of Diecke et al, Sci Rep. 2015, Jan. 28; 5:8081 (doi:10.1038/srep08081), hereby incorporated by reference in its entirety and specifically for the methods and reagents for the generation of the miPSCs.

See also, e.g., Burridge et al., PLoS One, 2011 6(4):18293, hereby incorporated by reference in its entirety and specifically for the methods outlined therein.

In some cases, the pluripotency of the cells is measured or confirmed as outlined herein, for example by assaying for reprogramming factors or by conducting differentiation reactions as outlined herein and in the Examples.

### C. Generation of Hypo-Immunogenic Pluripotent (HIP) Cells

Generating HIP cells from pluripotent cells is done with as few as three genetic changes, resulting in minimal disruption of cellular activity but conferring immunosilencing to the cells.

As discussed herein, one embodiment utilizes a reduction or elimination in the protein activity of MHC I and II (HLA I and II when the cells are human). This can be done by altering genes encoding their component. In one embodiment, the coding region or regulatory sequences of the gene are disrupted using CRISPR. In another embodiment, gene translation is reduced using interfering RNA technologies. The third change is a change in a gene that regulates susceptibility to macrophage phagocytosis, such as CD47 or SIRPα-Engager, and this is generally a “knock in” of a gene using viral technologies.

In some cases, where CRISPR is being used for the genetic modifications, hiPSC cells that contain a Cas9 construct that enable high efficiency editing of the cell line can be used; see, e.g., the Human Episomal Cas9 iPSC cell line, A33124, from Life Technologies.

### 1. HLA-I Reduction

The HIP cells of the invention include a reduction in MHC I function (HLA I when the cells are derived from human cells).

As will be appreciated by those in the art, the reduction in function can be accomplished in a number of ways, including removing nucleic acid sequences from a gene, interrupting the sequence with other sequences, or altering the regulatory components of the nucleic acid. For example, all or part of a coding region of the gene of interest can be removed or replaced with “nonsense” sequences, frameshift mutations can be made, all or part of a regulatory sequence such as a promoter can be removed or replaced, translation initiation sequences can be removed or replaced, etc.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A,B,C antibodies that bind to the the alpha chain of the human major histocompatibility HLA Class I antigens.

a. B2M Alteration

In one embodiment, the reduction in HLA-I activity is done by disrupting the expression of the β-2 microglobulin gene in the pluripotent stem cell, the human sequence of which is disclosed herein. This alteration is generally referred to herein as a gene “knock out”, and in the HIP cells of the invention it is done on both alleles in the host cell. Generally the techniques to do both disruptions is the same.

A particularly useful embodiment uses CRISPR technology to disrupt the gene. In some cases, CRISPR technology is used to introduce small deletions/insertions into the coding region of the gene, such that no functional protein is produced, often the result of frameshift mutations that result in the generation of stop codons such that truncated, non-functional proteins are made.

Accordingly, a useful technique is to use CRISPR sequences designed to target the coding sequence of the B2M gene in mouse or the B2M gene in human. After gene editing, the transfected iPSC cultures are dissociated to single cells. Single cells are expanded to full-size colonies and tested for a CRISPR edit by screening for presence of aberrant sequence from the CRISPR cleavage site. Clones with deletions in both alleles are picked.

Such clones did not express B2M as demonstrated by PCR and did not express HLA-I as demonstrated by FACS analysis (see examples 1 and 6, for example).

Assays to test whether the B2M gene has been inactivated are known and described herein. In one embodiment, the assay is a Western blot of cell lysates probed with antibodies to the B2M protein. In another embodiment, reverse transcriptase polymerase chain reactions (rt-PCR) confirms the presence of the inactivating alteration.

In addition, the cells can be tested to confirm that the HLA I complex is not expressed on the cell surface. This may be assayed by FACS analysis using antibodies to one or more HLA cell surface components as discussed above.

It is noteworthy that others have had poor results when trying to silence the B2M genes at both alleles. See, e.g. Gornalusse et al., Nature Biotech.

Doi/10.1038/nbt.3860). 2. HLA-II Reduction

In addition to a reduction in HLA I, the HIP cells of the invention also lack MHC II function (HLA II when the cells are derived from human cells).

As will be appreciated by those in the art, the reduction in function can be accomplished in a number of ways, including removing nucleic acid sequences from a gene, adding nucleic acid sequences to a gene, disrupting the reading frame, interrupting the sequence with other sequences, or altering the regulatory components of the nucleic acid. In one embodiment, all or part of a coding region of the gene of interest can be removed or replaced with “nonsense” sequences. In another embodiment, regulatory sequences such as a promoter can be removed or replaced, translation initiation sequences can be removed or replaced, etc.

The successful reduction of the MHC II function (HLA II when the cells are derived from human cells) in the pluripotent cells or their derivatives can be measured using techniques known in the art such as Western blotting using antibodies to the protein, FACS techniques, rt-PCR techniques, etc.

a. CIITA Alteration

In one embodiment, the reduction in HLA-II activity is done by disrupting the expression of the CIITA gene in the pluripotent stem cell, the human sequence of which is shown herein. This alteration is generally referred to herein as a gene “knock out.” In the HIP cells of the invention it is done on both alleles in the host cell.

Assays to test whether the CIITA gene has been inactivated are known and described herein. In one embodiment, the assay is a Western blot of cells lysates probed with antibodies to the CIITA protein. In another embodiment, reverse transcriptase polymerase chain reactions (rt-PCR) confirms the presence of the inactivating alteration.

In addition, the cells can be tested to confirm that the HLA II complex is not expressed on the cell surface. Again, this assay is done as is known in the art. Exemplary analyses include Western Blots or FACS analysis using commercial antibodies that bind to human HLA Class II HLA-DR, DP and most DQ antigens as outlined below.

A particularly useful embodiment uses CRISPR technology to disrupt the CIITA gene. CRISPRs were designed to target the coding sequence of the Ciita gene in mouse or the CIITA gene in human, an essential transcription factor for all MHC II molecules. After gene editing, the transfected iPSC cultures were dissociated into single cells.

They were expanded to full-size colonies and tested for successful CRISPR editing by screening for the presence of an aberrant sequence from the CRISPR cleavage site. Clones with deletions did not express CIITA as determined by PCR and did not express MHC II/HLA-II as determined by FACS analysis.

### 3. Phagocytosis Reduction

In addition to the reduction of HLA I and II (or MHC I and II), generally using B2M and CIITA knock-outs, the HIP cells of the invention have a reduced susceptibility to macrophage phagocytosis and NK cell killing. The resulting HIP cells “escape” the immune macrophage and innate pathways due to one or more CD47 transgenes.

a. CD47 Increase

In some embodiments, reduced macrophage phagocytosis and NK cell killing susceptibility results from increased CD47 on the HIP cell surface. This is done in several ways as will be appreciated by those of skill in the art using “knock in” or transgenic technologies. In some cases, increased CD47 expression results from one or more CD47 transgene.

Accordingly, in some embodiments, one or more copies of a CD47 gene is added to the HIP cells under control of an inducible or constitutive promoter, with the latter being preferred. In some embodiments, a lentiviral construct is employed as described herein or known in the art. CD47 genes may integrate into the genome of the host cell under the control of a suitable promoter as is known in the art.

The HIP cell lines were generated from B2M−/− CIITA−/− iPSCs. Cells containing lentivirus vectors expressing CD47 were selected using a Blasticidin marker. The CD47 gene sequence was synthesized and the DNA was cloned into the plasmid Lentivirus pLenti6/V5 with a blasticidin resistance (Thermo Fisher Scientific, Waltham, MA)

In some embodiments, the expression of the CD47 gene can be increased by altering the regulatory sequences of the endogenous CD47 gene, for example, by exchanging the endogenous promoter for a constitutive promoter or for a different inducible promoter. This can generally be done using known techniques such as CRISPR.

Once altered, the presence of sufficient CD47 expression can be assayed using known techniques such as those described in the Examples, such as Western blots, ELISA assays or FACS assays using anti-CD47 antibodies. In general, “sufficiency” in this context means an increase in the expression of CD47 on the HIP cell surface that silences NK cell killing. The natural expression levels on cells is too low to protect them from NK cell lysis once their MHC I is removed.

SIRPα engagement maybe be accomplished via engineered or alternative molecules. In some aspects of the invention, the engager molecule is a protein. In other aspects, the protein is a fusion protein. In other aspects, the fusion protein comprises a CD47 extracellular domain (ECD). In other aspects of the invention, the SIRP-α engager cell comprises an immunoglobulin superfamily domain. In other aspects of the invention, the engager molecule comprises an antibody Fab or a single chain variable fragment (scFV) that binds to SIRPa. In other aspects, the Fab or scFV binds to SIRPα with an affinity measured by its dissociation constant (Kd), wherein the Kd is between about 10⁻⁷ and 10⁻¹³ M. See PCT/US2021/062008, incorporated by reference herein in its entirety.

In some aspects of the invention, the engager molecule comprises one or more antibody complimentarity determining regions (CDRs) that binds to SIRPa.

### 4. Suicide Genes

In some embodiments, the invention provides hypoimmunogenic pluripotent cells that comprise a “suicide gene” or “suicide switch”. These are incorporated to function as a “safety switch” that can cause the death of the hypoimmunogenic pluripotent cells should they grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al., Mol. Therap. 20(10):1932-1943 (2012), Xu et al., Cell Res. 8:73-8 (1998), both incorporated herein by reference in their entirety.)

In other embodiments, the suicide gene is an inducible Caspase protein. An inducible Caspase protein comprises at least a portion of a Caspase protein capable of inducing apoptosis. In one embodiment, the portion of the Caspase protein is exemplified in SEQ ID NO:6. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, AP1903. Thus, the suicide function of iCasp9 in the instant invention is triggered by the administration of a chemical inducer of dimerization (CID). In some embodiments, the CID is the small molecule drug AP1903. Dimerization causes the rapid induction of apoptosis. (See WO2011146862; Stasi et al, N. Engl. J Med 365;18 (2011); Tey et al., Biol. Blood Marrow Transplant. 13:913-924 (2007), each of which are incorporated by reference herein in their entirety.)

### 5. CD16t, CD32t, or CD64t Expression

The cells of the invention have a reduced susceptibility to ADCC and CDC resulting from CD16t, CD32t, or CD64t expression. The resulting cells sequester antibodies due to the increased expression. In one embodiment, the cells comprise one or more CD16t, CD32t, or CD64t transgenes.

In some embodiments, reduced ADCC or CDC susceptibility results from CD16t, CD32t, or CD64t on the cell surface. This is done in several ways as will be appreciated by those in the art using “knock in” or transgenic technologies. In some cases, CD16t, CD32t, or CD64t expression results from one or more transgenes.

Accordingly, in some embodiments, one or more copies of a CD16t, CD32t, or CD64t gene is added to the cells under control of an inducible or constitutive promoter, with the latter being preferred. In some embodiments, a lentiviral construct is employed as described herein or known in the art. The genes may integrate into the genome of the host cell under the control of a suitable promoter as is known in the art.

Cells containing lentivirus vectors expressing CD16t, CD32t, or CD64t are selected using a Blasticidin marker. The gene sequence is synthesized and the DNA may be cloned, for instance, into the plasmid Lentivirus pLenti6/V5 with a blasticidin resistance (Thermo Fisher Scientific, Waltham, MA)

In some embodiments, the expression of the gene can be increased by altering the regulatory sequences of the endogenous CD16, CD32, or CD64 gene combined with a truncation or mutation as described herein. This may be accomplished, for example, by exchanging the endogenous promoter for a constitutive promoter or for a different inducible promoter. This can generally be done using known techniques such as CRISPR.

Once altered, the presence of sufficient expression can be assayed using known techniques such as those described in the Examples, such as Western blots, ELISA assays or FACS assays using anti-CD16, CD32, or CD64 antibodies. In general, “sufficiency” in this context means an increase in expression the cell surface that sequesters antibodies and inhibis ADCC or CDC.

### 6. Assays for HIP Phenotypes and Retention of Pluripotency

Once the HIP cells have been generated, they may be assayed for their hypo-immunogenicity and/or retention of pluripotency as is generally described herein and in the examples.

For example, hypo-immunogenicity are assayed using a number of techniques One exemplary technique includes transplantation into allogeneic hosts and monitoring for HIP cell growth (e.g. teratomas) that escape the host immune system. HIP derivatives are transduced to express luciferase and can then followed using bioluminescence imaging. Similarly, the T cell and/or B cell response of the host animal to the HIP cells are tested to confirm that the HIP cells do not cause an immune reaction in the host animal. T cell function is assessed by Elispot, Elisa, FACS, PCR, or mass cytometry (CYTOF). B cell response or antibody response is assessed using FACS or luminex. Additionally, or alternatively, the cells may be assayed for their ability to avoid innate immune responses, e.g. NK cell killing. NK cell lytolytic activity is assessed in vitro or in vivo using techniques known in the art.

Similarly, the retention of pluripotency is tested in a number of ways. In one embodiment, pluripotency is assayed by the expression of certain pluripotency-specific factors as generally described herein. Additionally or alternatively, the HIP cells are differentiated into one or more cell types as an indication of pluripotency.

D. Generation of HIPO− CD16t, CD32t, or CD64t-expressing Cells

In some aspects of the invention, the HIP cells generated as above will already be HIPO− cells because the process will have started with pluripotent cells having an 0-blood type.

Other aspects of the invention involve the enzymatic conversion of A and B antigens. In preferred aspects, the B antigen is converted to 0 using an enzyme. In more preferred aspects, the enzyme is an a-galactosidase. This enzyme eliminates the terminal galactose residue of the B antigen. Other aspects of the invention involve the enzymatic conversion of A antigen to 0. In preferred aspects, the A antigen is converted to 0 using an a-N-acetylgalactosaminidase. Enzymatic conversion is discussed, e.g., in Olsson et al., Transfusion Clinique et Biologique 11:33-39 (2004); U.S. Pat. Nos. 4,427,777, 5,606,042, 5,633,130, 5,731,426, 6,184,017, 4,609,627, and 5,606,042; and Int'l Pub. No. WO9923210, each of which are incorporated by reference herein in their entirety.

Other embodiments of the invention involve genetically engineering the cells by knocking out the ABO gene Exon 7 or silencing the SLC14A1 (JK) gene. Other embodiments of the invention involve knocking out the C and E antigens of the Rh blood group system (RH), K in the Kell system (KEL), Fya and Fy3 in the Duffy system (FY), Jkb in the Kidd system (JK), or U and S in the MNS blood group system. Any knockout methodology known in the art or described herein, such as CRISPR, talens, or homologous recombination, may be employed.

### E. Preferred Embodiments of the Invention

The CD16t, CD32t, or CD64t expressing HIP, HIPO−, iPSC, iPSCO−, ESC, or ESCO− cells, or derivatives thereof, of the invention may be used to treat, for example, Type 1 diabetes, cardiac diseases, neurological diseases, cancer, blindness, vascular diseases, and others that respond to regenerative medicine therapies. In particular, the invention contemplates using the cells for differentiation into any cell type. Thus, provided herein are cells that express CD16t, CD32t, or CD64t and exhibit pluripotency but do not result in a host ADCC or CDC response when transplanted into an allogeneic host such as a human patient, either as pluripotent cells or as the differentiated products of them. Additionally, the CD16, CD32, or CD64 intracellular signaling function is reduced or eliminated by truncation or mutation.

In one aspect, the cells of the present invention comprise a nucleic acid encoding a chimeric antigen receptor (CAR), with CD16t, CD32t, or CD64t expression. The CAR can comprise an extracellular domain, a transmembrane domain, and an intracellular signaling domain.

In some embodiments, the extracellular CAR domain binds to an antigen selected from the group consisting of CD19, CD20, CD22, CD38, CD123, CS1, CD171, BCMA, MUC16, ROR1, and WT1. In certain embodiments, the extracellular domain comprises a single chain variable fragment (scFv). In some embodiments, the CAR-Transmembrane domain comprises CD3ζ, CD4, CD8α, CD28, 4-1BB, OX40, ICOS, CTLA-4, PD-1, LAG-3, and BTLA. In certain embodiments, the CAR intracellular signaling domain comprises CD3ζ, CD28, 4-1BB, OX40, ICOS, CTLA-4, PD-1, LAG-3, and BTLA.

In certain embodiments, the CAR comprises an anti-CD19 scFv domain, a CD28 transmembrane domain, and a CD3 zeta signaling intracellular domain. In some embodiments, the CAR comprises anti-CD19 scFv domain, a CD28 transmembrane domain, a 4-1BB signaling intracellular domain, and a CD3 zeta signaling intracellular domain.

In another aspect of the invention, an isolated CAR-T cell that expresses CD16t, CD32t, or CD64t is produced by in vitro differentiation of any one of the cells described herein. In some embodiments, the cell is a cytotoxic hypoimmune CAR-T cell.

In various embodiments, the in vitro differentiation comprises culturing the cell carrying a CAR construct in a culture media comprising one or more growth factors or cytokines selected from the group consisting of bFGF, EPO, Flt3L, IGF, IL-3, IL-6, IL-15, GM-CSF, SCF, and VEGF. In some embodiments, the culture media further comprises one or more selected from the group consisting of a BMP activator, a GSK3 inhibitor, a ROCK inhibitor, a TGFβ receptor/ALK inhibitor, and a NOTCH activator.

In particular embodiments, the isolated CAR-T cell of the invention produced by in vitro differentiation is used as a treatment of cancer.

Another aspect of the invention provides is a method of treating a patient with cancer by administering a composition comprising a therapeutically effective amount of any of the isolated CAR-T cells described herein. In some embodiments, the composition further comprises a therapeutically effective carrier.

In some embodiments, the administration step comprises intravenous administration, subcutaneous administration, intranodal administration, intratumoral administration, intrathecal administration, intrapleural administration, and intraperitoneal administration. In certain instances, the administration further comprises a bolus or by continuous perfusion.

In some embodiments, the cancer is a blood cancer selected from the group consisting of leukemia, lymphoma, and myeloma. In various embodiments, the cancer is a solid tumor cancer or a liquid tumor cancer.

In another aspect, the present invention provides a method of making any one of the isolated CAR-T/CD16t, CD32t, or CD64t cells described herein. The method includes in vitro differentiating of any one of the cells of the invention wherein in vitro differentiating comprises culturing them in a culture media comprising one or more growth factors or cytokines selected from the group consisting of bFGF, EPO, Flt3L, IGF, IL-2, IL-3, IL-6, IL-7, IL-15, GM-CSF, SCF, and VEGF. In some embodiments, the culture media further comprises one or more selected from the group consisting of a BMP activator, a GSK3 inhibitor, a ROCK inhibitor, a TGFβ receptor/ALK inhibitor, and a NOTCH activator.

In some embodiments, the in vitro differentiation comprises culturing the HIPO-cells on feeder cells. In other embodiments, the in vitro differentiating comprises culturing in simulated microgravity. In certain instances, the culturing in simulated microgravity is for at least 72 hours.

In some aspects provided herein is an isolated, engineered hypoimmune cardiac cell (hypoimmunogenic cardiac cell) differentiated from a cell that expresses CD16t, CD32t, or CD64t.

In some aspects, provided herein is a method of treating a patient suffering from a heart condition or disease. The method comprises administering a composition comprising a therapeutically effective amount of a population of any one of the isolated, engineered hypoimmune cardiac cells derived from cells of the invention as described herein. In some embodiments, the composition further comprises a therapeutically effective carrier.

In some embodiments, the administration comprises implantation into the patient's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the heart condition or disease is selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease.

In some aspects, provided herein is a method of producing a population of hypoimmune cardiac cells from a population of HIPO−/CD16t, CD32t, or CD64t cells by in vitro differentiation, wherein endogenous β-2 microglobulin (B2M) gene activity and endogenous class II transactivator (CIITA) gene activity have been eliminated and expression of CD47 or another SIRPα-Engager protein has been increased in the HIPO-cells. The method comprises: (a) culturing a population of HIPO-cells in a culture medium comprising a GSK inhibitor; (b) culturing the population of HIPO-cells in a culture medium comprising a WNT antagonist to produce a population of pre-cardiac cells; and (c) culturing the population of pre-cardiac cells in a culture medium comprising insulin to produce a population of hypoimmune cardiac cells.

In some embodiments, the GSK inhibitor is CHIR-99021, a derivative thereof, or a variant thereof. In some instances, the GSK inhibitor is at a concentration ranging from about 2 μM to about 10 μM. In some embodiments, the WNT antagonist is IWR1, a derivative thereof, or a variant thereof. In some instances, the WNT antagonist is at a concentration ranging from about 2 μM to about 10 μM.

In some aspects, provided herein is an isolated, engineered CD16, CD32, or CD64-overexpressing endothelial cell differentiated from HIPO− cells. In other aspects, the isolated, engineered endothelial cell of the invention is selected from the group consisting of a capillary endothelial cell, vascular endothelial cell, aortic endothelial cell, brain endothelial cell, and renal endothelial cell.

In some aspects, provided herein is a method of treating a patient suffering from a vascular condition or disease. In some embodiments, the method comprises administering a composition comprising a therapeutically effective amount of a population of isolated, engineered endothelial cells of the invention.

The method comprises administering a composition comprising a therapeutically effective amount of a population of any one of the isolated, engineered CD16, CD32, or CD64-overexpressing endothelial cells described herein. In some embodiments, the composition further comprises a therapeutically effective carrier or excipient. In some embodiments, the administration comprises implantation into the patient's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the vascular condition or disease is selected from the group consisting of, vascular injury, cardiovascular disease, vascular disease, ischemic disease, myocardial infarction, congestive heart failure, hypertension, ischemic tissue injury, limb ischemia, stroke, neuropathy, and cerebrovascular disease.

In some aspects, provided herein is a method of producing a population of hypoimmune endothelial cells from a population of CD16t, CD32t, or CD64t-expressing cells by in vitro differentiation, wherein endogenous β-2 microglobulin (B2M) gene activity and endogenous class II transactivator (CIITA) gene activity have been eliminated and CD47 expression has been increased in the HIPO-cells. The method comprises: (a) culturing a population of HIPO-cells in a first culture medium comprising a GSK inhibitor; (b) culturing the population of HIPO-cells in a second culture medium comprising VEGF and bFGF to produce a population of pre-endothelial cells; and (c) culturing the population of pre-endothelial cells in a third culture medium comprising a ROCK inhibitor and an ALK inhibitor to produce a population of hypoimmune endothelial cells.

In some embodiments, the GSK inhibitor is CHIR-99021, a derivative thereof, or a variant thereof. In some instances, the GSK inhibitor is at a concentration ranging from about 1 μM to about 10 μM. In some embodiments, the ROCK inhibitor is Y-27632, a derivative thereof, or a variant thereof. In some instances, the ROCK inhibitor is at a concentration ranging from about 1 μM to about 20 μM. In some embodiments, the ALK inhibitor is SB-431542, a derivative thereof, or a variant thereof. In some instances, the ALK inhibitor is at a concentration ranging from about 0.5 μM to about 10 μM.

In some embodiments, the first culture medium comprises from 2 μM to about 10 μM of CHIR-99021. In some embodiments, the second culture medium comprises 50 ng/ml VEGF and 10 ng/ml bFGF. In other embodiments, the second culture medium further comprises Y-27632 and SB-431542. In various embodiments, the third culture medium comprises 10 μM Y-27632 and 1 μM SB-431542. In certain embodiments, the third culture medium further comprises VEGF and bFGF. In particular instances, the first culture medium and/or the second medium is absent of insulin.

In some aspects, provided herein is an isolated, engineered hypoimmune dopaminergic neuron (DN) differentiated from a CD16t, CD32t, or CD64t-expressing cell.

In some embodiments, the endogenous β-2 microglobulin (B2M) gene activity and endogenous class II transactivator (CIITA) gene activity have been eliminated, CD47 or another SIRPα-Engager expression has been increased, the neuron is blood type O and Rh−.

In some embodiments, the isolated dopaminergic neuron is selected from the group consisting of a neuronal stem cell, neuronal progenitor cell, immature dopaminergic neuron, and mature dopaminergic neuron.

In some aspects, provided herein is a method of treating a patient suffering from a neurodegenerative disease or condition. In some embodiments, the method comprises administering a composition comprising a therapeutically effective amount of a population of any one of the isolated hypoimmune dopaminergic neurons. In some embodiments, the composition further comprises a therapeutically effective carrier. In some embodiments, the population of the isolated hypoimmune dopaminergic neurons is on a biodegradable scaffold.

The administration may comprise transplantation or injection. In some embodiments, the neurodegenerative disease or condition is selected from the group consisting of Parkinson's disease, Huntington disease, and multiple sclerosis.

In some aspects, provided herein is a method of producing a population of CD16t, CD32t, or CD64t-expressing dopaminergic neurons by in vitro differentiation. In some embodiments, the endogenous β-2 microglobulin (B2M) gene activity and endogenous class II transactivator (CIITA) gene activity have been eliminated, CD47 or another SIRPα-Engager expression has been increased, the blood group is O and Rh−. In some embodiments, the method comprises (a) culturing the population of cells in a first culture medium comprising one or more factors selected from the group consisting of sonic hedgehog (SHH), BDNF, EGF, bFGF, FGF8, WNT1, retinoic acid, a GSK3β inhibitor, an ALK inhibitor, and a ROCK inhibitor to produce a population of immature dopaminergic neurons; and (b) culturing the population of immature dopaminergic neurons in a second culture medium that is different than the first culture medium to produce a population of dopaminergic neurons.

In some embodiments, the GSKO inhibitor is CHIR-99021, a derivative thereof, or a variant thereof. In some instances, the GSKO inhibitor is at a concentration ranging from about 2 μM to about 10 μM. In some embodiments, the ALK inhibitor is SB-431542, a derivative thereof, or a variant thereof. In some instances, the ALK inhibitor is at a concentration ranging from about 1 μM to about 10 μM. In some embodiments, the first culture medium and/or second culture medium are absent of animal serum.

In some embodiments, the method also comprises isolating the population of hypoimmune dopaminergic neurons from non-dopaminergic neurons. In some embodiments, the method further comprises cryopreserving the isolated population of hypoimmune dopaminergic neurons.

In some aspects, provided herein is an isolated engineered hypoimmune pancreatic islet cell differentiated from a CD16t, CD32t, or CD64t-expressing cell. In some embodiments, the endogenous β-2 microglobulin (B2M) gene activity and endogenous class II transactivator (CIITA) gene activity have been eliminated, CD47 or another SIRPα-Engager expression has been increased, the blood type is O and Rh−.

In some embodiments, the isolated hypoimmune pancreatic islet cell is selected from the group consisting of a pancreatic islet progenitor cell, immature pancreatic islet cell, and mature pancreatic islet cell.

In some aspects, provided herein is a method of treating a patient suffering from diabetes. The method comprises administering a composition comprising a therapeutically effective amount of a population of any one of the isolated CD16t, CD32t, or CD64t-expressing pancreatic islet cells described herein. In some embodiments, the composition further comprises a therapeutically effective carrier. In some embodiments, the population of the isolated hypoimmune pancreatic islet cells is on a biodegradable scaffold.

In some instances, the administration comprises transplantation or injection.

In some aspects, provided herein is a method of producing a population of hypoimmune pancreatic islet cells from a population of CD16t, CD32t, or CD64t-expressing cells by in vitro differentiation. In some embodiments, the endogenous β-2 microglobulin (B2M) gene activity and endogenous class II transactivator (CIITA) gene activity have been eliminated, CD47 or another SIRPα-Engager expression has been increased, the blood type is O and Rh-in the HIPO− cells. The method comprises: (a) culturing the population of CD16t, CD32t, or CD64t-expressing cells in a first culture medium comprising one or more factors selected from the group consisting insulin-like growth factor (IGF), transforming growth factor (TGF), fibroblast growth factor (EGF), epidermal growth factor (EGF), hepatocyte growth factor (HGF), sonic hedgehog (SHH), and vascular endothelial growth factor (VEGF), transforming growth factor-β(TGFβ) superfamily, bone morphogenic protein-2 (BMP2), bone morphogenic protein-7 (BMP7), a GSK3β inhibitor, an ALK inhibitor, a BMP type 1 receptor inhibitor, and retinoic acid to produce a population of immature pancreatic islet cells; and (b) culturing the population of immature pancreatic islet cells in a second culture medium that is different than the first culture medium to produce a population of hypoimmune pancreatic islet cells.

In some embodiments, the GSK inhibitor is CHIR-99021, a derivative thereof, or a variant thereof. In some instances, the GSK inhibitor is at a concentration ranging from about 2 μM to about 10 μM. In some embodiments, the ALK inhibitor is SB-431542, a derivative thereof, or a variant thereof. In some instances, the ALK inhibitor is at a concentration ranging from about 1 μM to about 10 μM. In some embodiments, the first culture medium and/or second culture medium are absent of animal serum.

In some embodiments, the method also comprises isolating the population of CD16t, CD32t, or CD64t-expressing pancreatic islet cells from non-pancreatic islet cells. In some embodiments, the method further comprises cryopreserving the isolated population of hypoimmune pancreatic islet cells.

In some aspects, provided herein is an isolated, engineered hypoimmune retinal pigmented epithelium (RPE) cell differentiated from a CD16t, CD32t, or CD64t-expressing cell, wherein endogenous β-2 microglobulin (B2M) gene activity and endogenous class II transactivator (CIITA) gene activity have been eliminated, CD47 or another SIRPα-Engager expression has been increased, the blood type is O and Rh−.

In some embodiments, the isolated hypoimmune RPE cell is selected from the group consisting of a RPE progenitor cell, immature RPE cell, mature RPE cell, and functional RPE cell.

In some aspects, provided herein is a method of treating a patient suffering from an ocular condition. The method comprises administering a composition comprising a therapeutically effective amount of a population of any one of a population of the isolated CD16t, CD32t, or CD64t-expressing RPE cells described herein. In some embodiments, the composition further comprises a therapeutically effective carrier. In some embodiments, the population of the isolated hypoimmune RPE cells is on a biodegradable scaffold. In some embodiments, the administration comprises transplantation or injection to the patient's retina.

In some embodiments, the ocular condition is selected from the group consisting of wet macular degeneration, dry macular degeneration, juvenile macular degeneration, Leber's Congenital Ameurosis, retinitis pigmentosa, and retinal detachment.

In some aspects, provided herein is a method of producing a population of CD16t, CD32t, or CD64t-expressing retinal pigmented epithelium (RPE) cells from a population of cells by in vitro differentiation. In some embodiments, the endogenous β-2 microglobulin (B2M) gene activity and endogenous class II transactivator (CIITA) gene activity have been eliminated and CD47 or another SIRPα-Engager expression has been increased in the HIPO-cells. The method comprises: (a) culturing the population of HIPO− cells in a first culture medium comprising any one of the factors selected from the group consisting of activin A, bFGF, BMP4/7, DKK1, IGF1, noggin, a BMP inhibitor, an ALK inhibitor, a ROCK inhibitor, and a VEGFR inhibitor to produce a population of pre-RPE cells; and (b) culturing the population of pre-RPE cells in a second culture medium that is different than the first culture medium to produce a population of hypoimmune RPE cells.

In some embodiments, the ALK inhibitor is SB-431542, a derivative thereof, or a variant thereof. In some instances, the ALK inhibitor is at a concentration ranging from about 2 μM to about 10 μM. In some embodiments, the ROCK inhibitor is Y-27632, a derivative thereof, or a variant thereof. In some instances, the ROCK inhibitor is at a concentration ranging from about 1 μM to about 10 μM.

In some embodiments, the first culture medium and/or second culture medium lack animal serum.

In some embodiments, the method further comprises isolating the population of hypoimmune RPE cells from non-RPE cells. In some embodiments, the method further comprises cryopreserving the isolated population of hypoimmune RPE cells.

In one aspect, human pluripotent stem cells (PSCs) resist ADCC or CDC by CD16t, CD32t, or CD64t-expressing. In some embodiments, they are hypoimmune pluripotent stem cells (hiPSC). They are rendered hypo-immunogenic by a) the disruption of the B2M gene at each allele (e.g. B2M −/−), b) the disruption of the CIITA gene at each allele (e.g. CIITA −/−), and c) by the overexpression of the CD47 gene (CD47+, e.g. through introducing one or more additional copies of the CD47 gene or activating the genomic gene).

This renders the hiPSC population B2M−/− CIITA −/− CD47tg. In a preferred aspect, the cells are non-immunogenic. In another embodiment, the HIP cells are rendered non-immunogenic B2M−/− CIITA −/− CD47tg as described above but are further modified by including an inducible suicide gene that is induced to kill the cells in vivo when required. In other aspects, CD16, CD32, or CD64-overexpressing HIPO cells are created when HIP cells are rendered blood type O by by knocking out the ABO gene Exon 7 or silencing the SLC14A1 (JK) gene and the cells are rendered Rh-by knocking out the C and E antigens of the Rh blood group system (RH), K in the Kell system (KEL), Fya and Fy3 in the Duffy system (FY), Jkb in the Kidd system (JK), or U and S in the MNS blood group system.

### F. Maintenance of HIPO−/CD16t, CD32t, or CD64t Cells

Once generated, the HIPO-CD16t, CD32t, or CD64t cells can be maintained in an undifferentiated state as is known for maintaining iPSCs. For example, HIP cells are cultured on Matrigel using culture media that prevents differentiation and maintains pluripotency.

### G. Differentiation of HIPO−/CD16t, CD32t, or CD64t Cells

The invention provides HIPO−/CD16t, CD32t, or CD64t cells that are differentiated into different cell types for subsequent transplantation into subjects. As will be appreciated by those in the art, the methods for differentiation depend on the desired cell type using known techniques. The cells are differentiated in suspension and then put into a gel matrix form, such as matrigel, gelatin, or fibrin/thrombin forms to facilitate cell survival. Differentiation is assayed as is known in the art, generally by evaluating the presence of cell-specific markers.

In some embodiments, the HIPO−/CD16t, CD32t, or CD64t cells are differentiated into hepatocytes to address loss of the hepatocyte functioning or cirrhosis of the liver. There are a number of techniques that can be used to differentiate HIPO− cells into hepatocytes; see for example Pettinato et al., doi:10.1038/spre32888, Snykers et al., Methods Mol Biol 698:305-314 (2011), Si-Tayeb et al, Hepatology 51:297-305 (2010) and Asgari et al., Stem Cell Rev (:493-504 (2013), all of which are hereby expressly incorporated by reference in their entirety and specifically for the methodologies and reagents for differentiation. Differentiation is assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release and glycogen storage.

In some embodiments, the HIPO−/CD16t, CD32t, or CD64t cells are differentiated into beta-like cells or islet organoids for transplantation to address type I diabetes mellitus (T1DM). Cell systems are a promising way to address T1DM, see, e.g., Ellis et al., doi/10.1038/nrgastro.2017.93, incorporated herein by reference. Additionally, Pagliuca et al. reports on the successful differentiation of 0-cells from hiPSCs (see doi/10.106/j.cell.2014.09.040, hereby incorporated by reference in its entirety and in particular for the methods and reagents outlined there for the large-scale production of functional human 0 cells from human pluripotent stem cells). Furthermore, Vegas et al. shows the production of human R cells from human pluripotent stem cells followed by encapsulation to avoid immune rejection by the host; (doi:10.1038/nm.4030, hereby incorporated by reference in its entirety and in particular for the methods and reagents outlined there for the large-scale production of functional human R cells from human pluripotent stem cells).

Differentiation is assayed as is known in the art, generally by evaluating the presence of R cell associated or specific markers, including but not limited to, insulin.

Differentiation can also be measured functionally, such as measuring glucose metabolism, see generally Muraro et al, doi:10.1016/j.cels.2016.09.002, hereby incorporated by reference in its entirety, and specifically for the biomarkers outlined there.

Once the dHIPO−/CD16t, CD32t, or CD64t beta cells are generated, they can be transplanted (either as a cell suspension or within a gel matrix as discussed herein) into the portal vein/liver, the omentum, the gastrointestinal mucosa, the bone marrow, a muscle, or subcutaneous pouches.

In some embodiments, the HIPO−/CD16t, CD32t, or CD64t cells are differentiated into retinal pigment epithelium (RPE) to address sight-threatening diseases of the eye. Human pluripotent stem cells have been differentiated into RPE cells using the techniques outlined in Kamao et al., Stem Cell Reports 2014:2:205-18, hereby incorporated by reference in its entirety and in particular for the methods and reagents outlined there for the differentiation techniques and reagents; see also Mandai et al., doi:10.1056/NEJMoa1608368, also incorporated in its entirety for techniques for generating sheets of RPE cells and transplantation into patients.

Differentiation can be assayed as is known in the art, generally by evaluating the presence of RPE associated and/or specific markers or by measuring functionally. See for example Kamao et al., doi:10.1016/j.stemcr.2013.12.007, hereby incorporated by reference in its entirety and specifically for the markers outlined in the first paragraph of the results section.

In some embodiments, the HIPO−/CD16t, CD32t, or CD64t cells are differentiated into cardiomyocytes to address cardiovascular diseases. Techniques are known in the art for the differentiation of hiPSCs to cardiomyoctes and discussed in the Examples. Differentiation can be assayed as is known in the art, generally by evaluating the presence of cardiomyocyte associated or specific markers or by measuring functionally; see for example Loh et al., doi:10.1016/j.cell.2016.06.001, hereby incorporated by reference in its entirety and specifically for the methods of differentiating stem cells including cardiomyocytes.

In some embodiments, the HIPO−/CD16t, CD32t, or CD64t cells are differentiated into endothelial colony forming cells (ECFCs) to form new blood vessels to address peripheral arterial disease. Techniques to differentiate endothelial cells are known.

See, e.g., Prasain et al., doi:10.1038/nbt.3048, incorporated by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the HIPO−/CD16t, CD32t, or CD64t cells are differentiated into thyroid progenitor cells and thyroid follicular organoids that can secrete thyroid hormones to address autoimmune thyroiditis. Techniques to differentiate thyroid cells are known the art. See, e.g. Kurmann et al., doi:10.106/j.stem.2015.09.004, hereby expressly incorporated by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

### H. Transplantation of Differentiated HIPO−/CD16t, CD32t, or CD64t Cells

As will be appreciated by those in the art, the differentiated HIPO−/CD16t, CD32t, or CD64t derivatives are transplated using techniques known in the art that depend on both the cell type and the ultimate use of these cells. In general, the cells of the invention are transplanted either intravenously or by injection at particular locations in the patient. When transplanted at particular locations, the cells may be suspended in a gel matrix to prevent dispersion while they take hold.

In order that the invention described herein may be more fully understood, the following examples are set forth. It should be understood that these examples are for illustrative purposes only and are not to be construed as limiting this invention in any manner.

### VII. EXAMPLES

HIP and HIPO− cells are generated as disclosed in WO2018/132783, PCT/US19/42123, PCT/US19/42117, and Prov. Appl. Nos. 62/698,973, 62/698,978, 62/698,981, 62/698,984, 62/846,399, and 62/855,499. Protection from NK cell and ADCC killing when CD64 is overexpressed on pluripotent cells is disclosed in PCT/US20/55120.

Each of the foregoing are incorporated herein by reference in their entirety.

### Example 1: CD64t expression sequesters antibodies by the F_{c} Domain

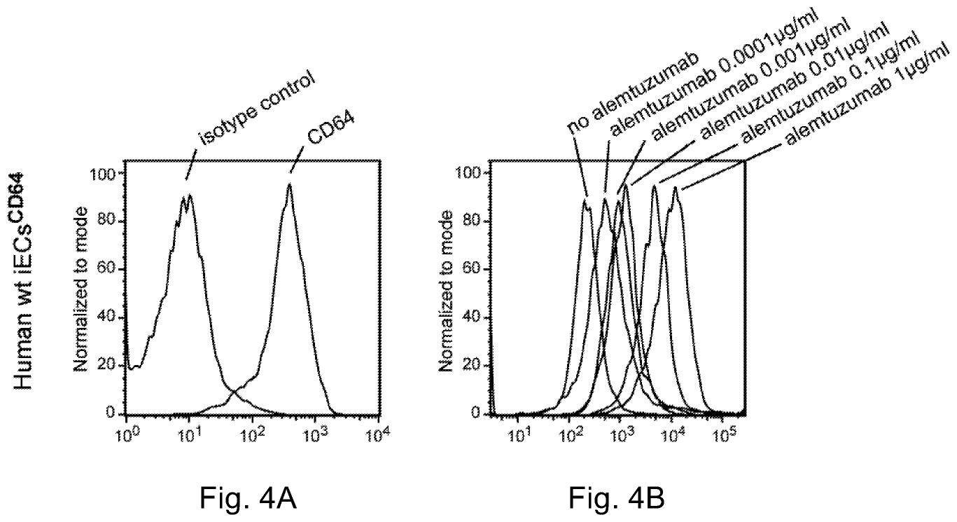

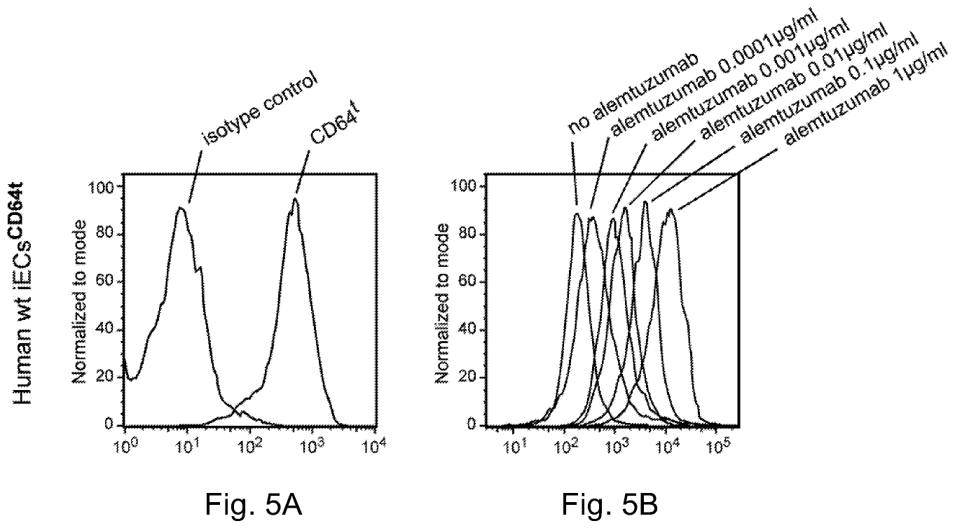

FIG. 3 is a schematic diagram showing a CD64t capturing free IgG F_{c} without inducing any signaling in the target cell. Human wild-type (wt) iPSC-derived endothelial cells (iECs) were transduced to express CD64 or CD64t. One hundred thousand iECs were plated one gelatin-coated six-well plates and incubated overnight at 37° C. at 5% CO₂. The next day, media was changed and 20 μl of lentiviral particles (at 1×10⁸ IFU/ml) carrying the transgene for truncated CD64 or 200 μl or lentiviral particles (at 1×10⁷ IFU/ml) carrying the full-length CD64 under a re-engineered EF1a promotor (custom product, GenTarget, San Diego, CA), was added to 1.5 ml media. After 36 h, 1 ml of cell media was added. After a further 24 h, a complete media change was performed. The CD64 or CD64t expression was measure by flow cytometry two days later (FIGS. 4A and 5A). Flow cytometry analyses for CD64 or CD64t were performed using a PE-tagged mouse anti-human CD64 antibody (BD Biosciences, San Jose, CA, Cat #558592). The antibody F_{c} binding capacity was measured using the humanized IgG1 anti-CD52 antibody alemtuzumab (Ichorbio, Wantage OX12 9FF, UK). This antibody does not recognize an epitope on the iPSC and only bound in the presence of CD64 or CD64t (FIGS. 4A and 5A, respectively). F_{c} binding was quantified using a QDot 655-tagged goat-anti-human IgG (H+L) F(ab′)2 secondary antibody (Catalog #Q-11221MP, Thermo Fisher Scientific, Carlsbad, CA). Flow cytometry showed increasing alemtuzumab F_{c} binding with increasing antibody concentrations for both CD64 and CD64t (FIGS. 4B and 5B, respectively).

### Example 2: HIP iECs expressing CD64t are protected from ADCC and CDC

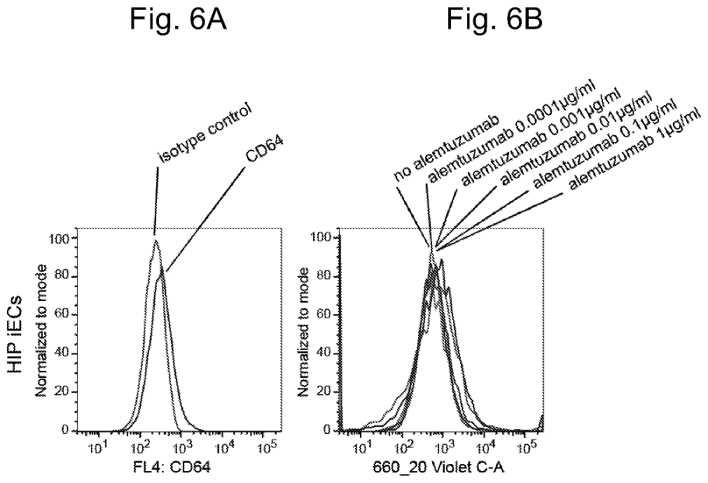

HIP iECs do not express CD64 (FIG. 6A). Since they also do not express any CD52, they do not bind anti-CD52 IgG1 (alemtuzumab) via their F_{ab} or their F_{c} (FIG. 6B).

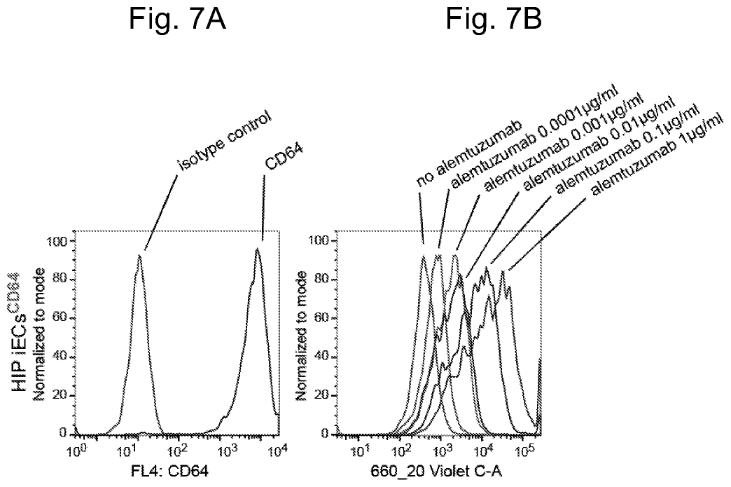

HIP iECs were transduced to express CD64 (FIG. 7A). Such HIP iECs^{CD64} were able to bind IgG1 via their F_{c} in a concentration-dependent fashion (FIG. 7B).

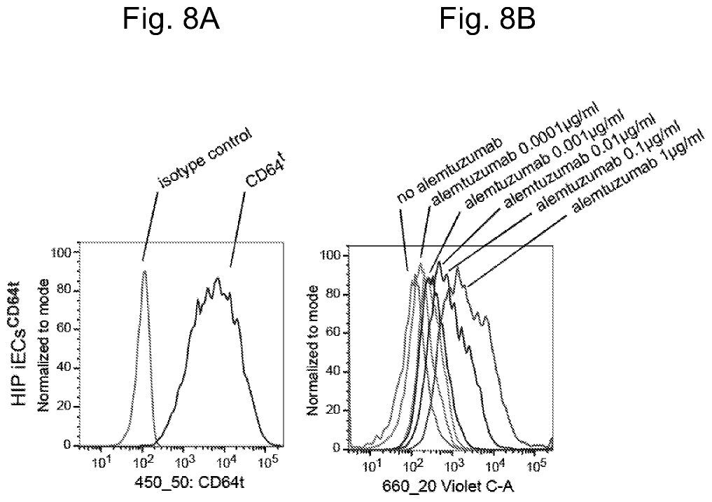

To avoid intracellular signaling, HIP iECs were transduced to express intracellularly truncated CD64 (CD64t) (FIG. 8A). Such HIP iECs^{CD64t} were able to bind IgG1 via their F_{c} in a concentration-dependent fashion (FIG. 8B).

HIP iECs and HIP iECs^{CD64t} were then transduced to additionally express CD52, the target for the highly cytotoxic IgG1 antibody alemtuzumab. Human primary NK cells were purchased from Stemcell Technologies (70036, Vancouver, Canada) and were cultured in RPMI-1640 plus 10% FCS hi and 1% pen/strep before performing the assays. In CDC assays, target cells were incubated with fresh ABO-compatible human serum.

Real-time killing assays were performed on the XCelligence SP platform and MP platform (ACEA Biosciences, San Diego, CA.). Special 96-well E-plates (ACEA Biosciences) coated with collagen (Sigma-Aldrich) were used. A total of 4×10⁴ human iECs, epiCs, beta cells, or CAR-T cells were plated in 100 μl medium. After the cell index reached 0.7, 4×10⁴ human NK cells was added to ADCC assays or 50 μl blood-type compatible human serum to CDC assays. Patient serum samples were treated with DTT to eliminate blood type IgM antibodies before use.

For Nalm6 killing assays, 4×10⁴ Nalm6 cells were plated and 4×10⁴ CAR-T cells were used as effector cells. The following antibodies were used and added after mixing with the NK cells in 50 μl medium or in the serum as indicated: humanized anti-CD52 IgG1 (alemtuzumab, ichorbio, catalog no. ICH4002), humanized anti-HLA-A2 IgG1 (clone 3PF12, Absolute Antibody, catalog no. AB00947-10.0), humanized anti-Rh(D) IgG1 (clone F5, Creative Biolabs, catalog no. FAMAB-0089WJ), humanized anti-TPO IgG1 (clone B8, Creative Biolabs, catalog no. FAMAB-0014JF), humanized anti-CD3 IgG1 (Creative Biolabs, custom product), humanized anti-CD19 scFv (FMC63) IgG1 (clone 136.20.1, Creative Biolabs, catalog no. HPAB-0440-YJ-m/h). Different concentrations ranging from 0.0001 μg/ml to 1 μg/ml were used. As a negative control, cells were treated with 2% Triton X-100 in medium (data not shown). Data were standardized and analyzed with the RTCA software (ACEA).

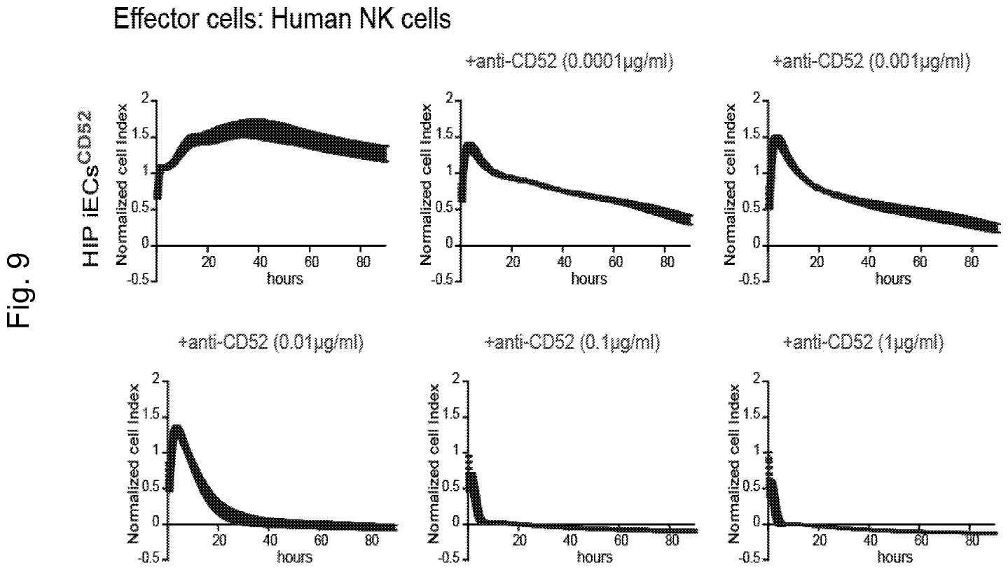

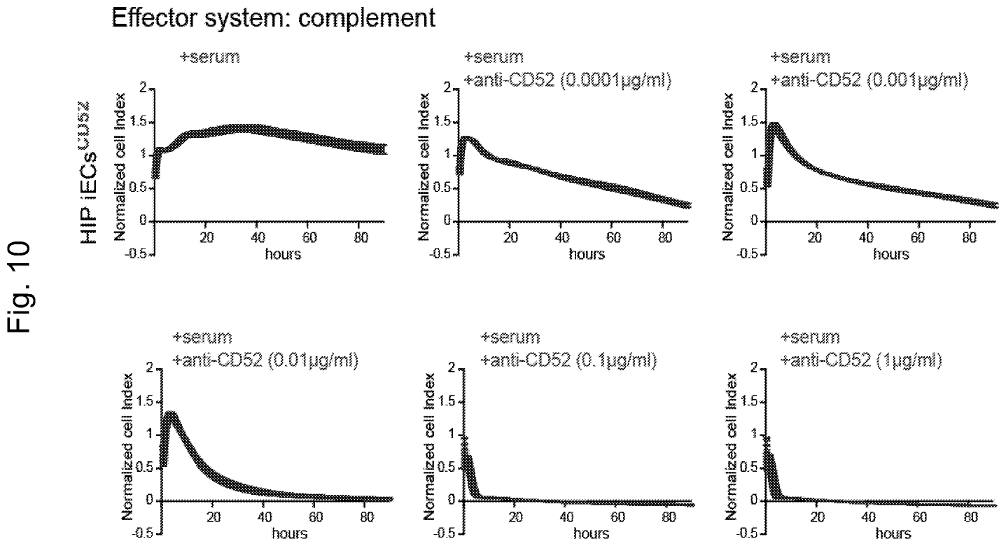

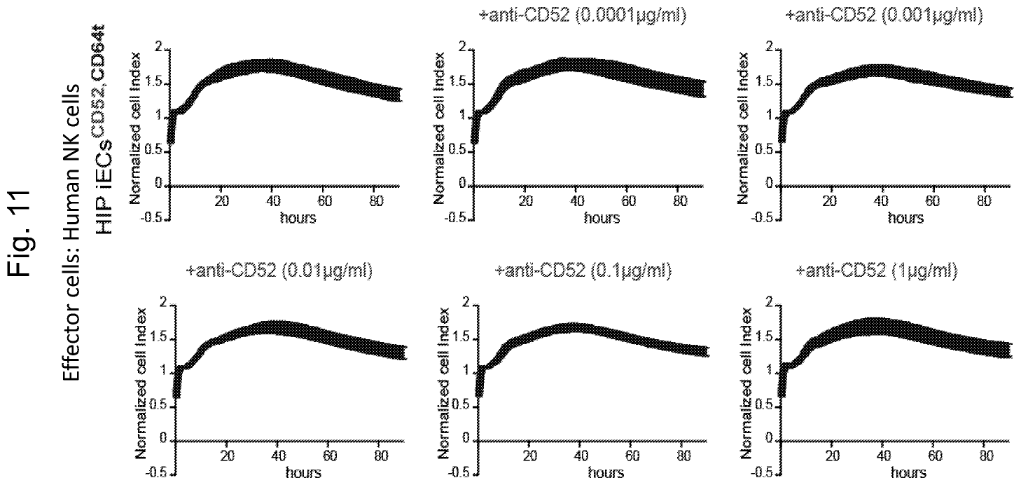

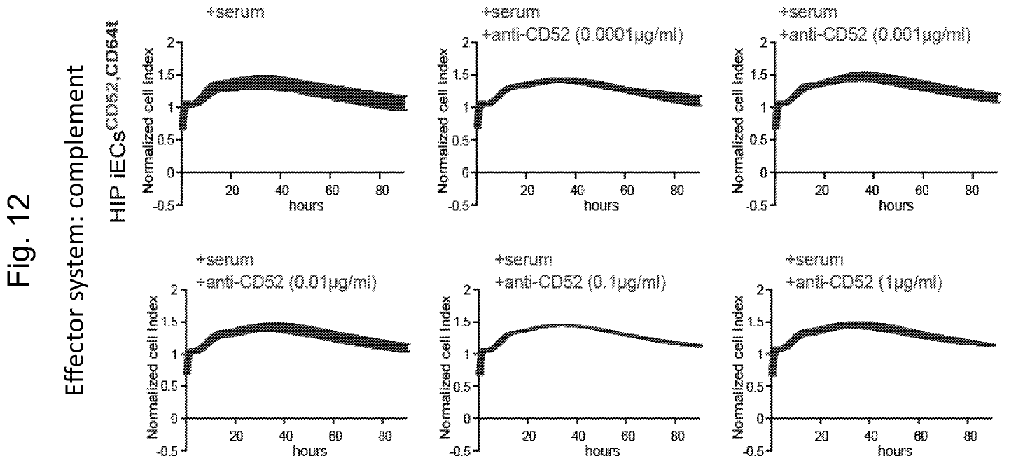

In ADCC and CDC killing assays, HIP iECs^{CD52} were killed by NK cells (FIG. 9) or complement (FIG. 10), respectively, in a concentration-dependent manner. HIP iECs^{CD52,CD64t} were found to be fully protected against both ADCC (FIG. 11) and CDC (FIG. 12).

### Example 3: Engineered human thyroid epithelial cells evade antibody-mediated autoimmune killing

Hashimoto's thyroiditis is a prototypic disease in which cytotoxic autoantibodies lead to the destruction of thyroid tissue. Anti-TPO antibodies, primarily of IgG1 subclass, are present at high concentrations in 90% of patients (Rapoport, B. & McLachlan, S. M., Thyroid autoimmunity. J Clin Invest 108, 1253-1259 (2001); Doullay, F. et al., Autoimmunity 9, 237-244 (1991)). They mediate both ADCC (Bogner, U. et al., J Cin EndocrinolMetab 59, 734-738 (1984); Stathatos, N. & Daniels, G. H., Autoimmune thyroid disease. Curr Opin Rheumatol 24, 70-75 (2012); Stassi, G. & De Maria, R. Autoimmune thyroid disease: new models of cell death in autoimmunity. Nat Rev Immunol 2, 195-204 (2002)) and CDC (Rebuffat, S. et al., J Cin Endocrinol Metab 93, 929-934 (2008); Chiovato, L. et al., J Clin Endocrinol Metab 77, 1700-1705 (1993)). This diminishes the function of the thyroid gland (Pearce, E. N., et al., NEngl J Med 348, 2646-2655 (2003)). The invention provides thyroid epithelial cells (epiCs) resistant to TPO antibodies for surviving innate immune responses and re-establishing organ function when transplanted into hypothyroid patients.

Immortalized human thyroid epithelial cells (epiCs, Inscreenex, Braunschweig, Germany, Catalog #INS-CI-1017) and primary human islet cells (Takara, Mountain View, CA, Catalog #Y10106) were used. Since the epiCs did not show a physiologic response to thyroid stimulating hormone (TSH) in vitro, their thyroid peroxidase (TPO) levels were well-below physiologic expression levels. Thus, a lentiviral transduction approach was used to artificially increase their TPO expression. One hundred thousand human thyroid epiCs were plated on gelatin-coated six-well plates and incubated overnight at 37° C. at 5% CO₂. The next day, media was changed and 200 μl of lentiviral particles (at 1×10⁷ IFU/ml) carrying the transgene for human TPO reengineered EF1a promotor (GenTarget) was added to 1.5 ml media. After 36 h, 1 ml of cell media was added.

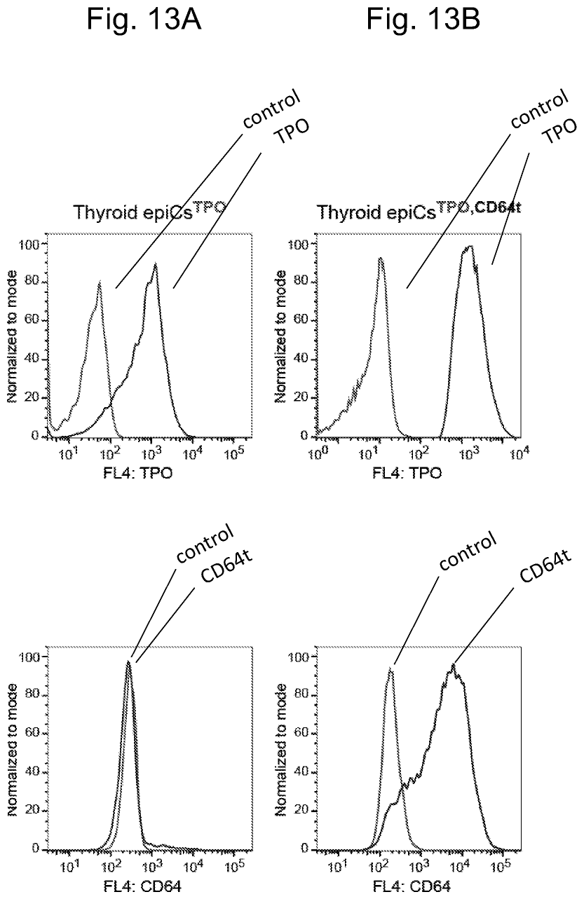

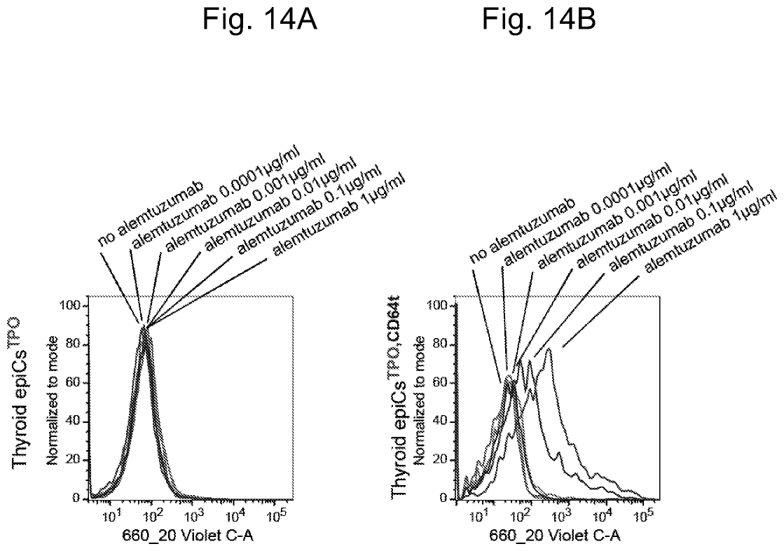

These epiCs^{TPO} showed good TPO expression but did not express CD64 (FIG. 13A). Then, epiCs^{TPO} were transduced to also express CD64t and epiCs^{TPO,CD64t} were generated (FIG. 13B). While thyroid epiCsr^{TPO} did not bind any human IgG1, even at high concentrations (FIG. 14A), epiCs^{TPO,CD64t} were effective in binding free human IgG1 F_{c} (FIG. 14B).

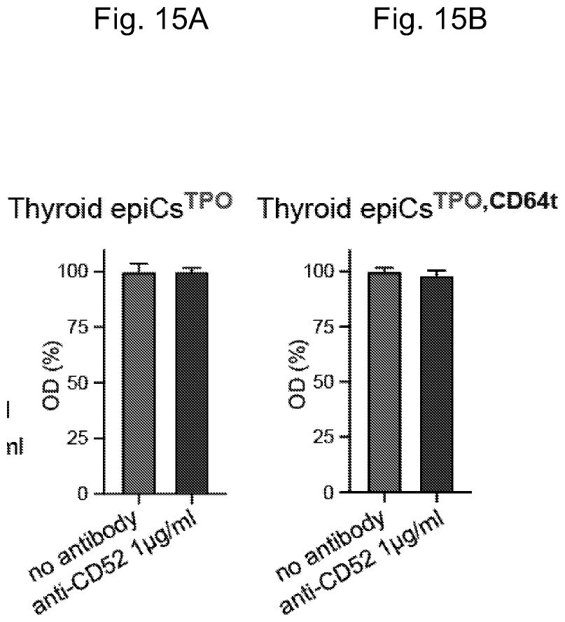

Thyroxine production was measured by ELISA. A 96 well plate was coated with gelatin and 3×10⁴ human thyroid epiCs^{TPO} or epiCs^{TPO,CD64t} per well were seeded in 100 μl h7H media and incubated for 24 hours at 370 C in 5% CO₂. The next day, the h7H media was changed and supplemented with 1 mU/mL native bovine thyroid stimulating hormone protein (TSH, catalog no. TSH-1315B, Creative Biomart, Shirley, NY). Three wells per epiC group were also supplemented with 1 μg/mL anti-CD52 IgG1 (alemtuzumab, clone Campath-1H, Biorad). After 72 hours, the supernatant was collected and the level of thyroxine was assessed using the thyroxine (T4) competitive ELISA kit (catalog no. EIAT4C, Invitrogen) according to manufacturer's instructions. Results are presented as change in optical density (OD) between groups with and without alemtuzumab. Both thyroid epiCs^{TPO} and epiCs^{TPO,CD64t} produced thyroxine irrespective of the presence of IgG1 antibodies (FIG. 15A and B).

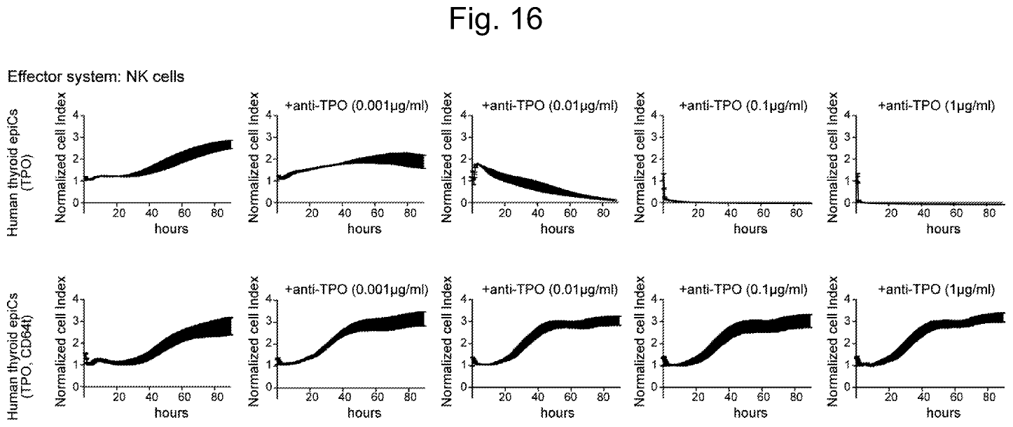

epiCs (TPO) with and without CD64t expression were plated on the XCelligence platform for in vitro impedance assays (FIG. 16). They attached to plastic dishes and were grow in a multi-layered fashion. Therefore, the cell index calculated by the XCelligence machine did not reach the steady-state levels known from endothelial cells that form even monolayers. The cell index kept increasing as these cells grew in multi-layer clusters. A rabbit-anti-TPO antibody (Abcam, Cambridge, MA, Catalog #ab203057) was used in increasing concentrations to induce antibody-mediated killing in this assay. 40,000 human NK cells were added as effector cells. Because rabbit Ig Fc does bind to human CD16, the epiCs (TPO) were killed in a concentration-dependent manner. The engineered epiCs (TPO, CD64t), however, effectively evaded antibody-mediated killing across all antibody concentrations used in this assay.

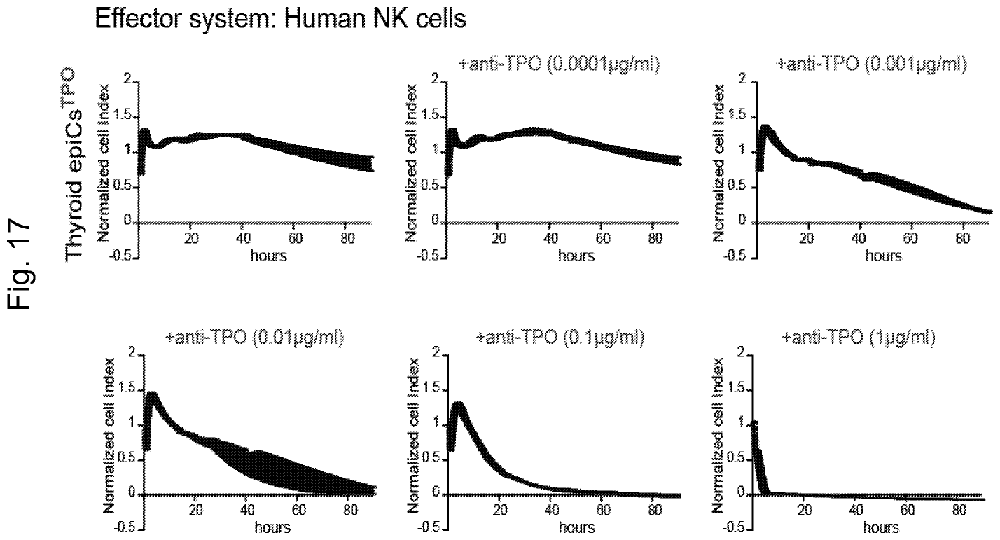

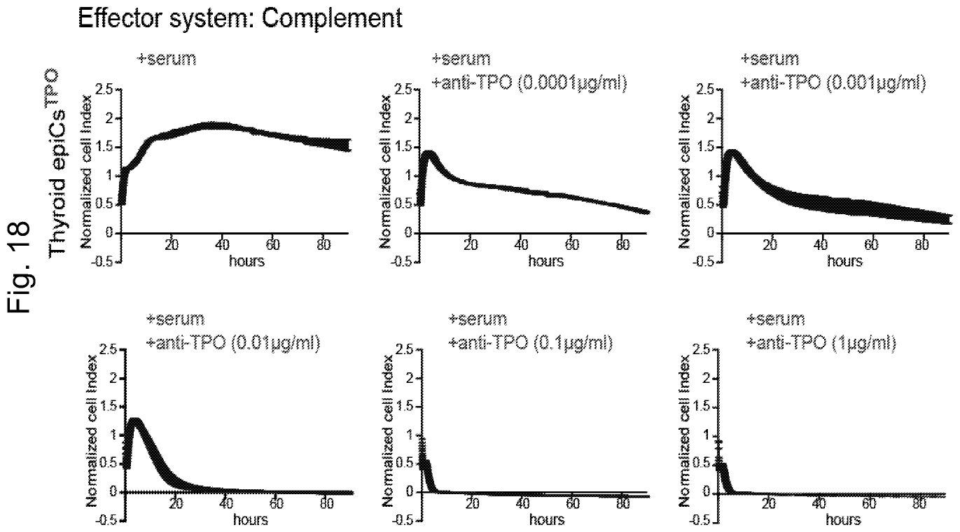

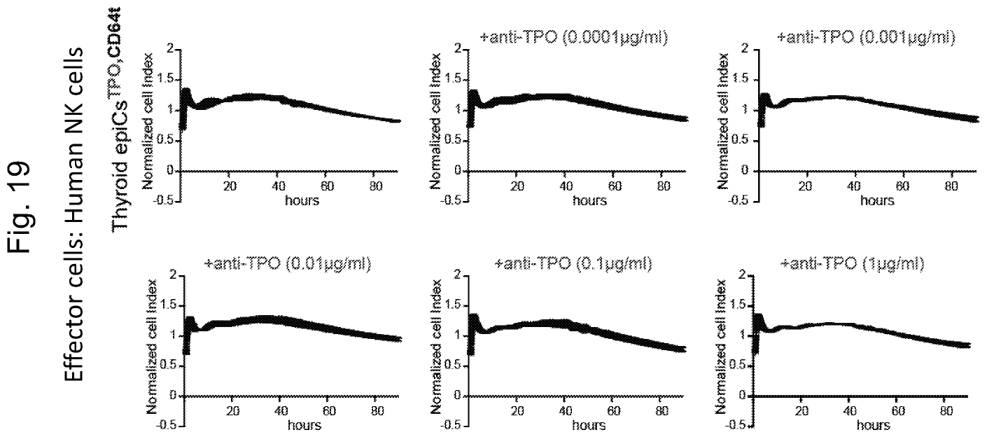

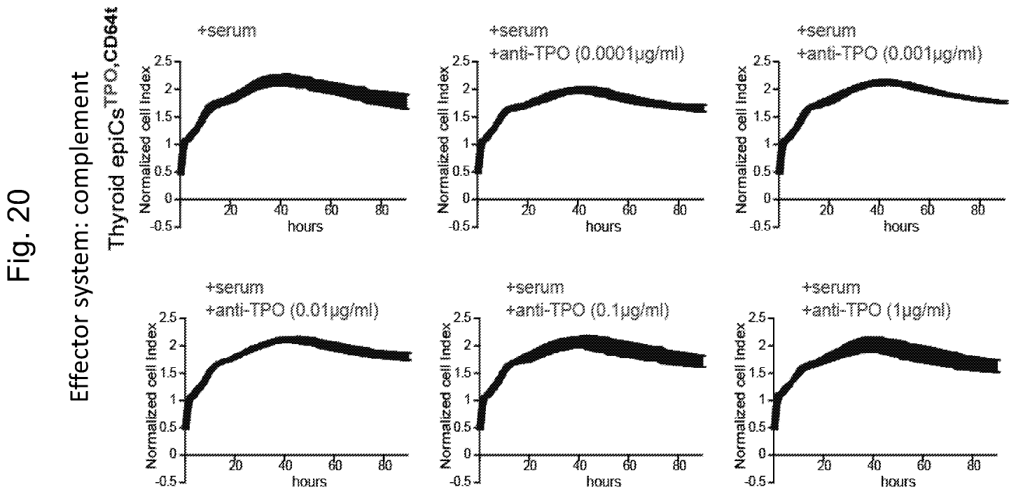

Although rabbit IgG F_{c} binds human CD64 and CD64t, we next used a humanized anti-TPO IgG1 antibody. This human IgG1 antibody was effective in killing thyroid epiCs^{TPO} in NK cell ADCC assays (FIG. 17) and CDC assays (FIG. 18). Thyroid epi^{TPO,CD64t}, however, were fully protected from ADCC (FIG. 19) and CDC(FIG. 20).

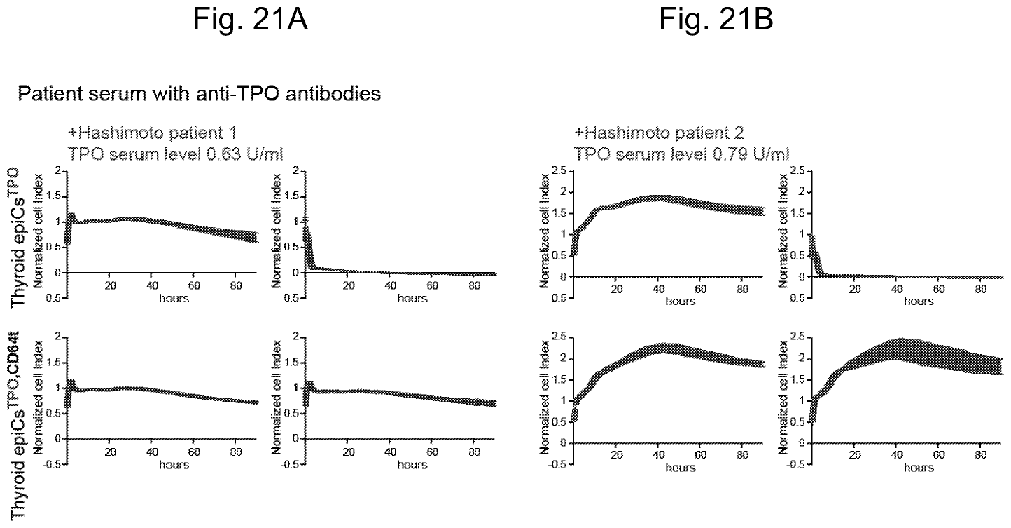

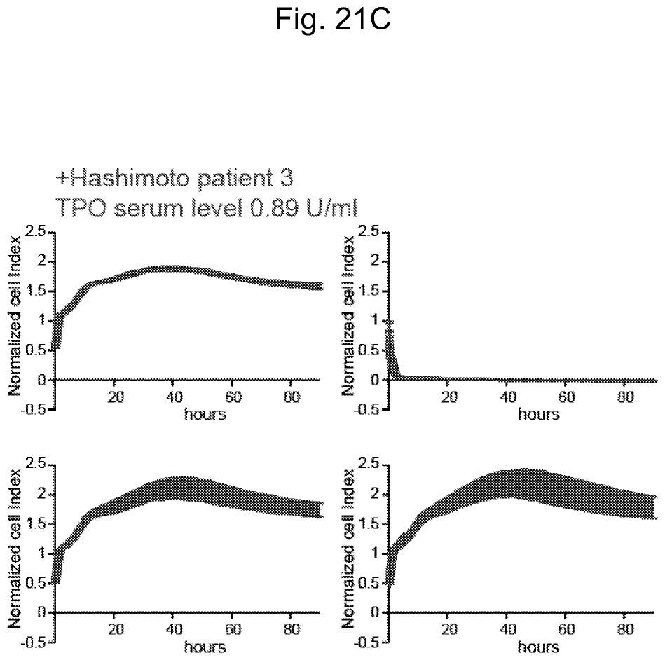

Serum samples from three patients with Hashimoto's thyroiditis and anti-TPO antibody titers 21-fold, 26.3-fold, and 29.6-fold the upper level of normal (0.03 U/ml) rapidly killed thyroid epiCs^{TPO} in CDC assays (FIG. 21A-C). Thyroid epi^{TPO,CD64t} were completely protected from killing in patient serum and thus withstood clinically relevant autoimmune conditions.

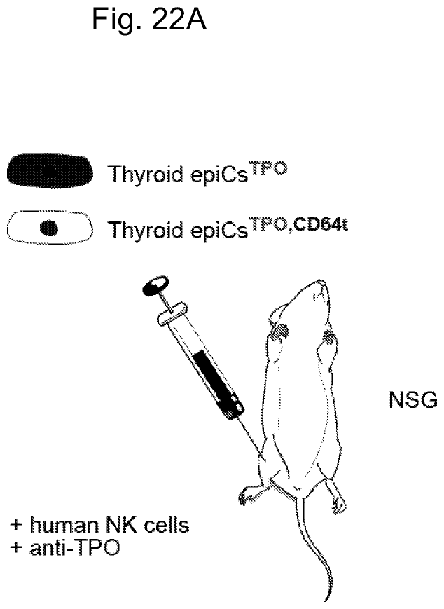

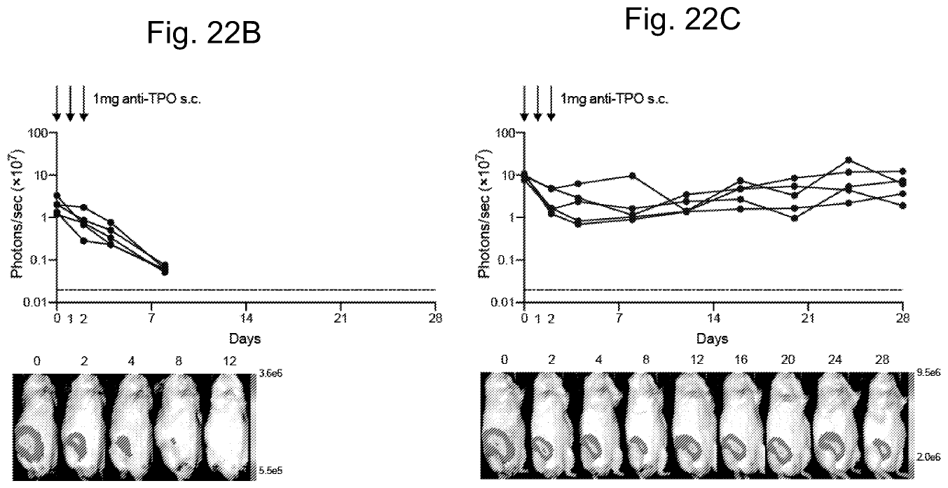

NSG (NOD.Cg-Prkdc^{scid}I12rg^{tm1WjI}/SzJ, 005557) mice 6-12 weeks old were purchased from the Jackson Laboratories (Sacramento, CA). The number of animals used in the examples is presented in each figure. NSG recipients were subcutaneously injected with 5×10⁴ epiCs^{TPO} or epiCs^{TPO,CD64t} one million human NK cells, and 1 mg doses of anti-TPO on days 0, 1, and 2 (FIG. 22A). All epiCs^{TPO} grafts vanished quickly (FIG. 22B), while all epiCs^{TPO,CD64t} grafts survived (FIG. 22C).

### Example 4: Engineered human beta cells evade HLA antibody-mediated killing

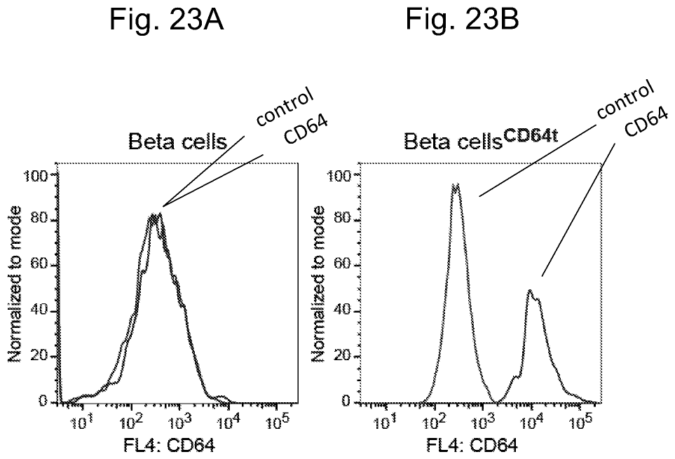

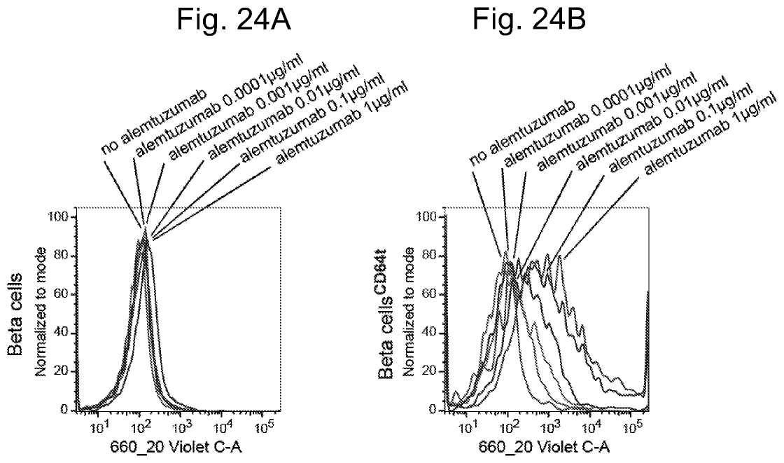

Type 1 diabetes mellitus (T1DM) is another autoimmune disease, which is T cell-mediated with an accompanying antibody response. We aimed to test whether CD64t expression can make them resistant against HLA antibody killing. Human iPSC-derived pancreatic beta cells were purchased from TaKaRa (ChiPSC22, catalog no. Y10106) and were cultured in Cellartis hiPS Beta Cell Media Kit (TaKaRa, catalog no. Y10108). Cells were plated in 12-well plates according to the manufacturer protocol. Some cells were transduced with CD64t lentiviral particles (GenTarget). Human iPSC-derived pancreatic islet cells (beta cells) do not express CD64 (FIG. 23A). Human iPSC-derived beta cells were then transduced to express CD64t (FIG. 23B). While beta cells were unable to bind IgG1 (FIG. 24A), beta cells^{CD64t} were able to capture and bind free IgG F_{c} (FIG. 24B).

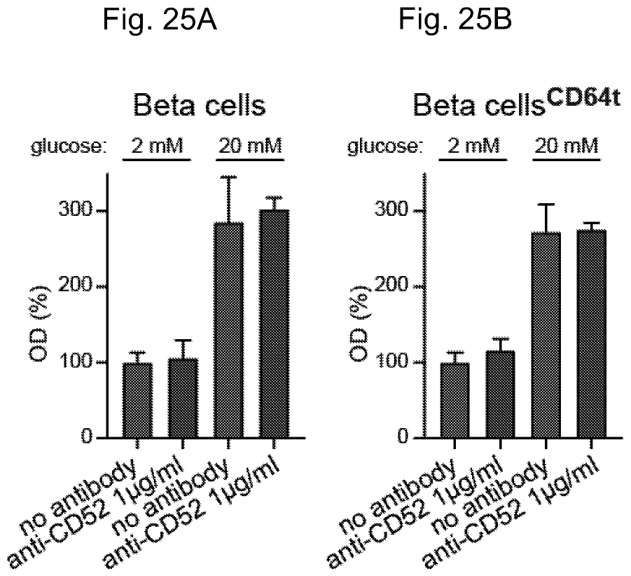

Insulin production was measured by ELISA. A 24 well plate was coated with gelatin and 5×10⁴ iPSC-derived beta cells and beta cells^{CD64t} cells (catalog no. Y10108, Takara Bio, San Jose, CA) per well were seeded in 500 μl Cellartis hiPS beta cell media and incubated for 24 hours at 370 C in 5% CO₂. The next day, the Cellartis hiPS beta cell media was changed to RPMI 1640 without glucose (catalog no. 11879-020, Gibco) for 2 hours. After 2 hours the media was changed to RPMI without glucose supplemented with 2 mM glucose (catalog no. G7528, Sigma Aldrich). Three wells per beta cell group were also supplemented with 1 μg/mL anti-CD52 IgG1 (alemtuzumab, clone Campath-1H, catalog no. MCA6101, Biorad). After 20 minutes, the supernatant was collected and the media was changed to RPMI without glucose supplemented with 20 mM glucose. Again, 1 μg/mL Alemtuzumab was added to 3 wells per group. After 20 minutes, the supernatant was collected and the level of human insulin was determined using the human insulin ELISA kit (catalog no. KAQ1251, Invitrogen) according to manufacturer's instructions. Results are presented as change in optical density (OD) between groups with and without alemtuzumab. Both beta cells (FIG. 25A) and beta cells^{CD64t} (FIG. 25B) showed intact glucose sensing and insulin production. These data show optical density (OD) measurements in an insulin ELISA. In low glucose medium (2 mM), both beta cells and beta cells^{CD64t} show lower insulin production compared to high glucose medium (20 mM). The insulin production was unchanged in the presence of l g/ml anti-CD52 (alemtuzumab) antibodies.

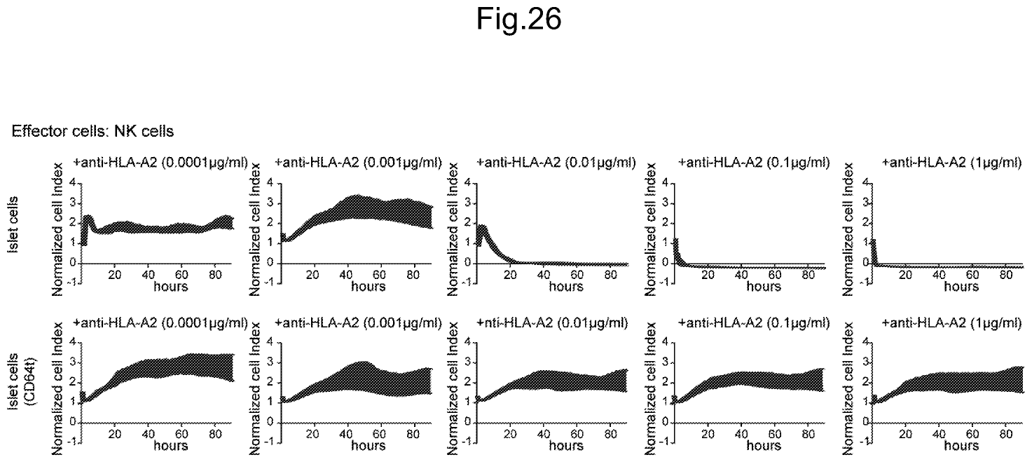

Islet cells (pancreatic beta cells) with and without CD64t expression were then plated on the XCelligence platform (FIG. 26). They attached to plastic dishes but also grew more in clusters and the cell index kept increasing over time. Despite the clustering growth pattern, the assay was still very sensitive in detecting target cell killing. The islet cells showed the HLA type A2. We thus used a humanized anti-human-HLA-A2 with human IgG1 F_{c} (Absolute Antibody, Boston, MA, Catalog #Ab00947-10.0) to induce antibody-mediated killing. Human NK cells (40,000) were used as effector cells. The islet cells were killed with increasing rapidity that corresponded with increasing anti-HLA-A2 concentrations. In contrast, the islet beta cells (CD64t) cells were protected from anti-HLA-A2 antibody-mediated killing.

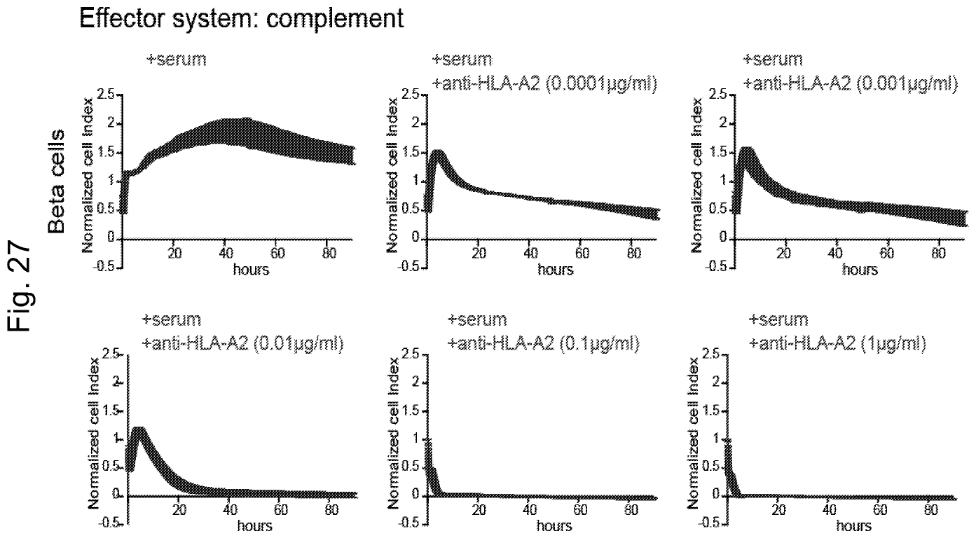

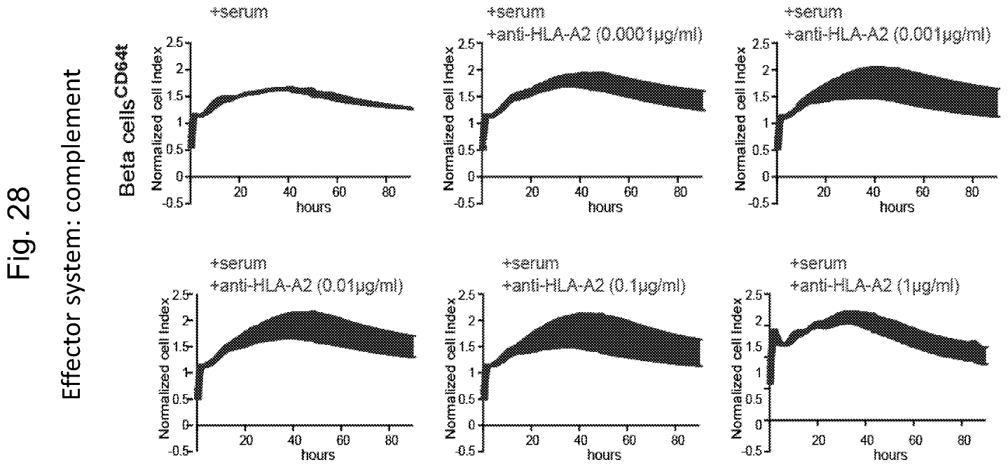

HLA-A2 expressing beta cells were killed increasingly quickly with increasing anti-HLA-A2 IgG1 antibody concentrations in CDC assays (FIG. 27). Beta cells^{CD64t}, however, were completely resistant against HLA antibody-mediated killing in CDC assays (FIG. 28).

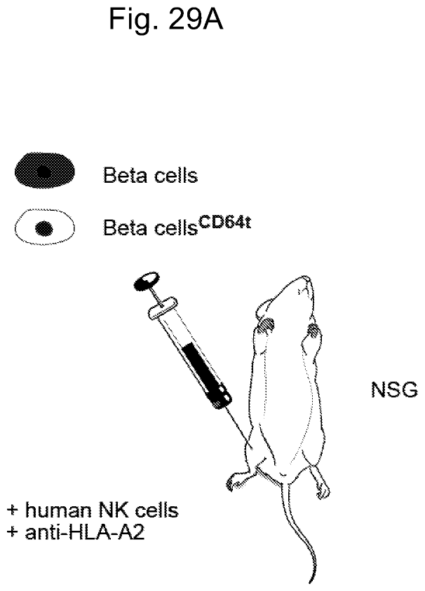

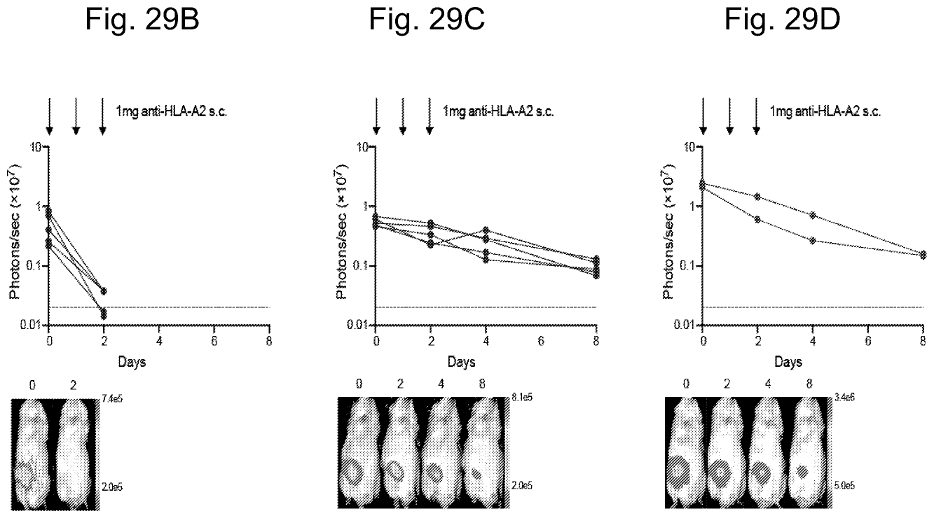

We then subcutaneously injected NSG mice with 5×10⁴ human beta cells or beta cells^{CD64t} together with one million human NK cells (FIG. 29A). Three 1 mg doses of anti-HLA-A2 were subcutaneously injected on days 0, 1, and 2. All injected cells were Luc⁺. For imaging, D-luciferin firefly potassium salt (375 mg/kg; Biosynth A G, Staad, Switzerland) was dissolved in PBS (pH 7.4, Gibco) and 250 μl was injected intraperitoneally in anesthetized mice. Animals were imaged in the AMI HT (Spectral Instruments Imaging). Region of interest (ROI) bioluminescence was quantified in units of maximum photons per second per centimeter square per steradian (p/s/cm²/sr). The maximum signal from an ROI was measured using Aura Image software (Spectral Instruments). All beta cell grafts vanished within 2 days (FIG. 29B), while all beta cell^{CD64t} grafts defied antibody-mediated killing (FIG. 29C) and paralleled the survival of beta cells without antibody challenge (FIG. 29D).

### Example 5: Engineered human CAR-T cells evade HLA-, non-HLA-, and CAR-directed antibody killing

Clinical CAR-T cell therapy induces an antibody response, which is even more pronounced in patients with solid tumors. Antibody protection was thus engineered into CAR-T cells. Human T cells were transduced to express a CD19 scFv-4-1BB-CD3ζ construct with or without additional CD64t expression. Human anti-CD19 CAR-T cells were generated from human PBMCs using lentiviral particles carrying a transgene for the CD19 scFv-4-1BB-CD3ζ construct (ProMab, catalog no. PM-CAR1002-V). PBMCs were stimulated with IL-2 overnight and seeded in 96-well U-bottom plates at a density of 10⁵ cells per well containing protamine sulfate and 1 μg/ml IL-2 (Peprotech). Lentiviruses were added to the wells at a MOI of 20. Some wells were transduced additionally with CD64t lentiviral particles at a MOI of 20 (GenTarget). Spinfection was carried out at 1800 rpm for 30 min at 25° C. After that, the cells were returned to a humidified 5% CO₂ incubator overnight. The medium was changed after 2 days and cells were seeded at a density of 10⁶ per ml in T cell media (OpTmizer, Thermo Fisher Scientific). CD3/CD28 beads (Thermo Fisher Scientific) were used for T-cell expansion. Cells were sorted for CAR⁺ and CAR⁺/C64t⁺ populations on BD Aria Fusion and used for further assays.

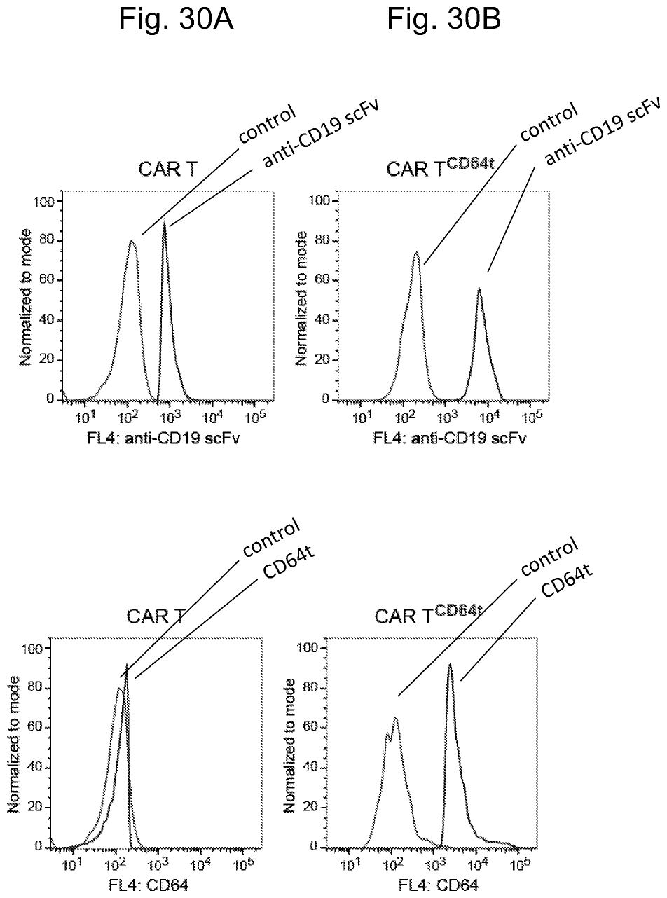

CAR-T cells showed marked expression of the CAR receptor (anti-CD19 scFv) but did not express CD64t (FIG. 30A). CAR-T cells were then transduced to express CD64t and such CAR-T^{CD64t} still expressed the CAR and additionally expressed CD64t (FIG. 30B).

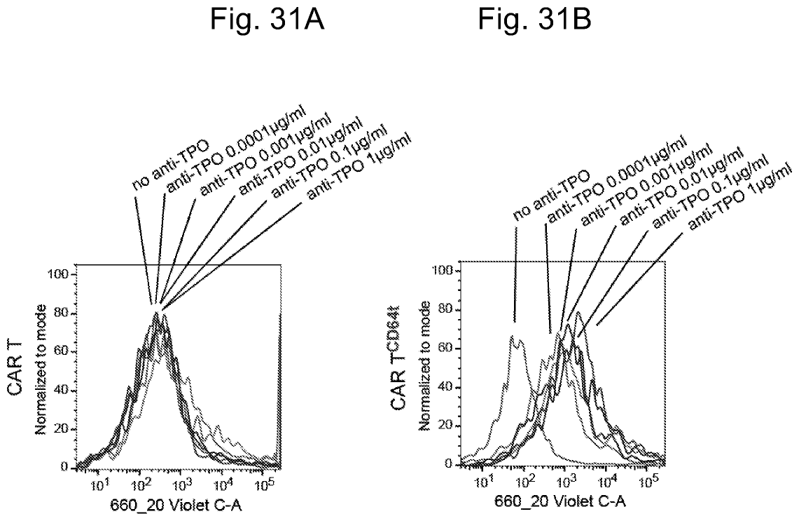

While CAR-T cells did not bind any free IgG1 (using an anti-TPO antibody, FIG. 31A), CAR-T^{CD64t} were able to capture and bind free IgG1 F_{c} (FIG. 31B).

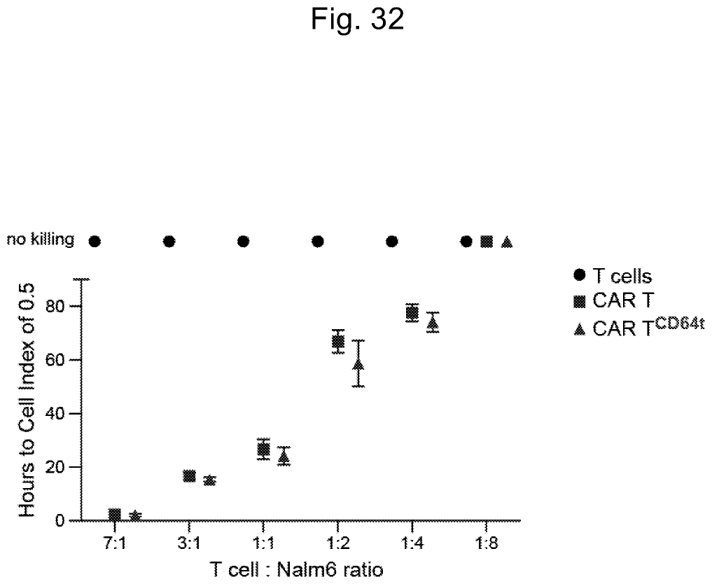

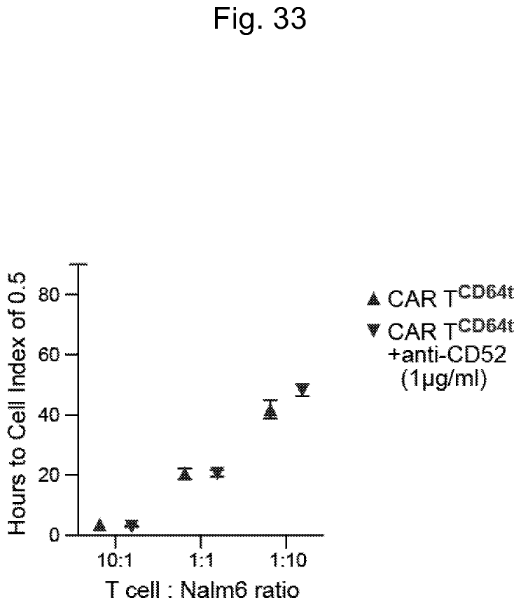

The killing efficacy of CAR-T, CAR-T^{CD64t}, and regular T cells was assessed against CD19-positive NALM target cells (FIG. 32). While control T cells showed no killing, both CAR-T and CAR-T^{CD64t} were equally effective killers across a wide range of effector cell-to-target cell ratios. Furthermore, the tumor killing capacity of CAR-T^{CD64t} was not affected by the presence of Fe-bound antibodies (FIG. 33).

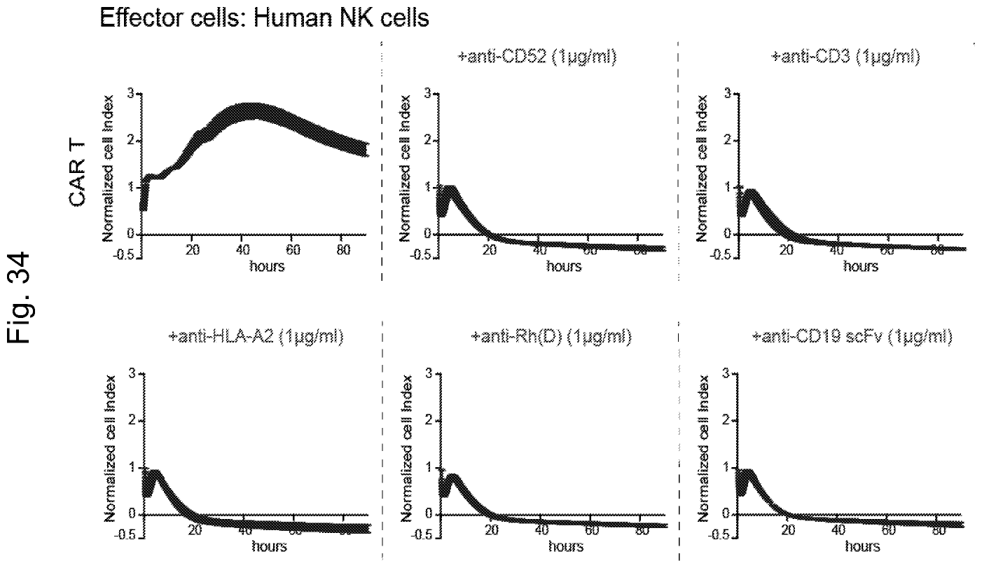

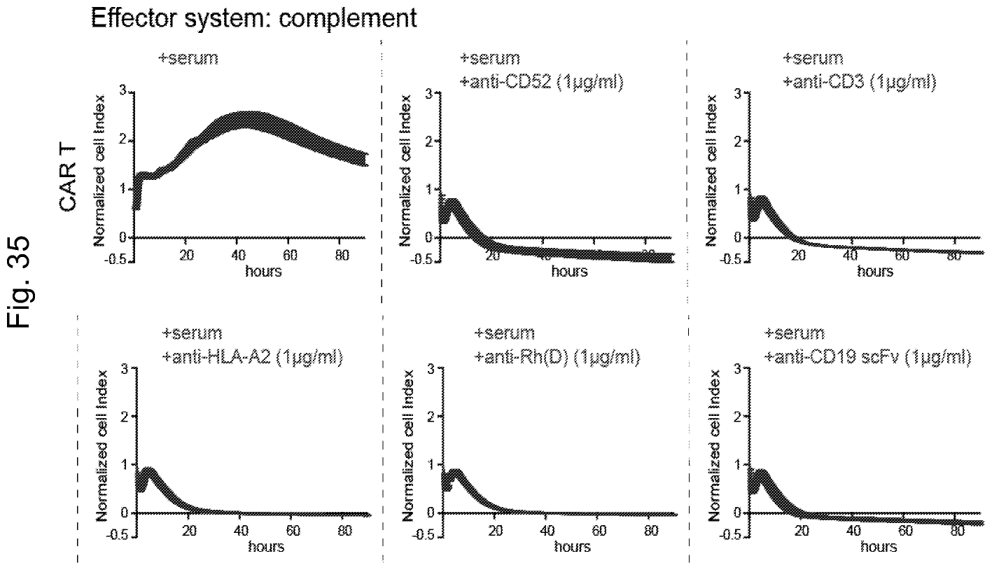

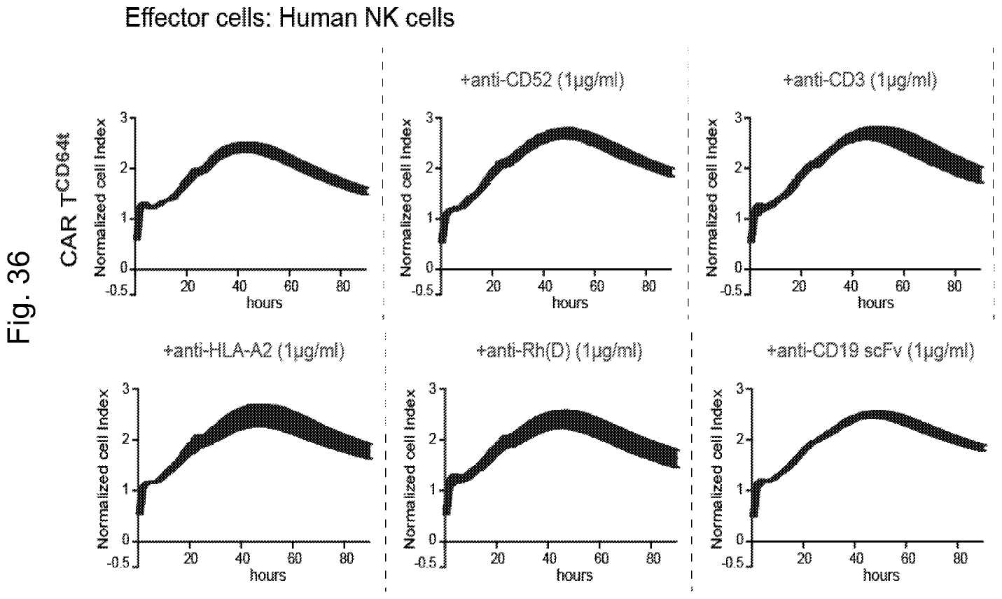

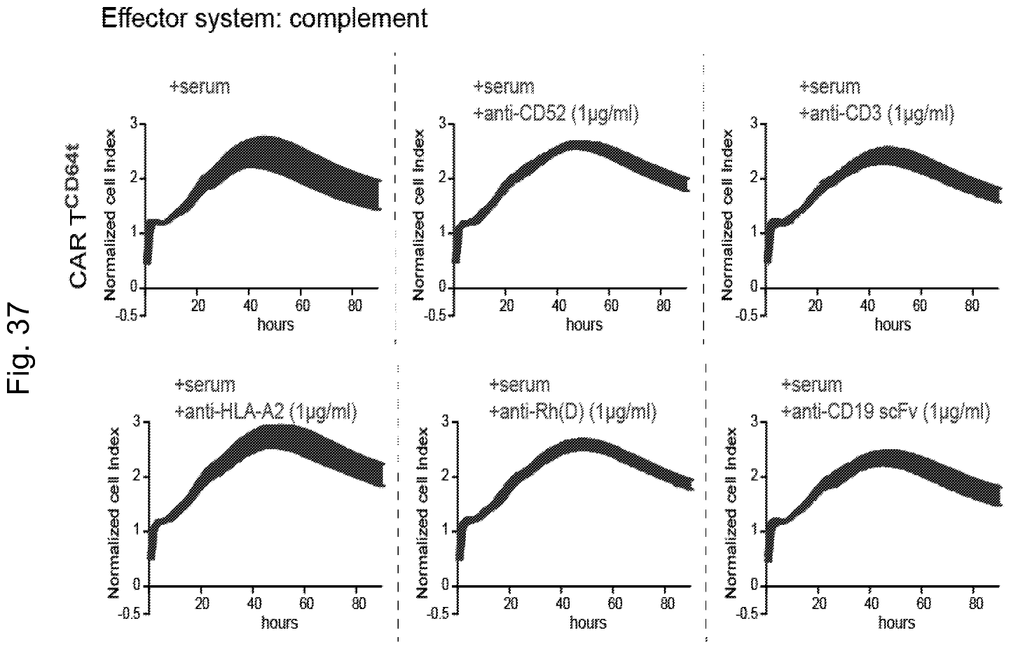

ADCC (FIG. 34) and CDC assays (FIG. 35) with cytotoxic antibodies against an HLA epitope (HLA-A2), non-HLA epitopes (CD52, CD3), a blood type antigen (Rh(D)), and the CAR receptor (anti-CD19 scFv) were performed, and the CAR-T cells were killed in all assays. CAR-T^{CD64t}, however, were able to evade antibody-mediated killing with all five antibodies in both ADCC (FIG. 36) and CDC assays (FIG. 37). CD64t expression does not affect the cytotoxicity of human CAR-T cells but makes them resistant against antibodies irrespective of their specificities.

### Example 6: Engineered human NK cells evade ADCC and CDC killing

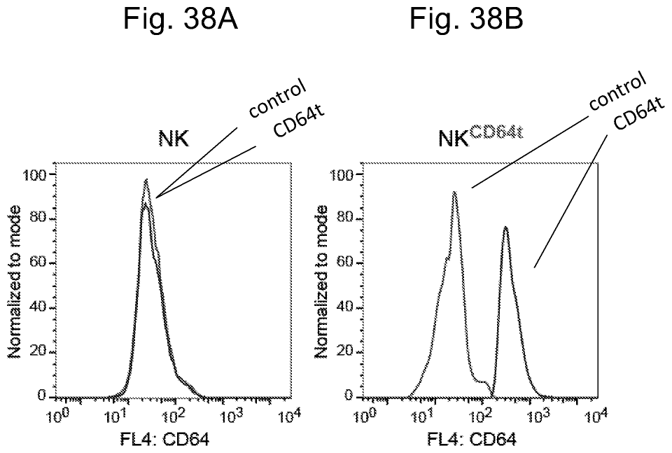

To show that antibody protection can be engineered into NK cells, human NK cells were transduced to express CD64t. Peripheral human NK cells do not express CD64t (FIG. 38A). NK^{CD64t} showed high CD64t expression (FIG. 38B).

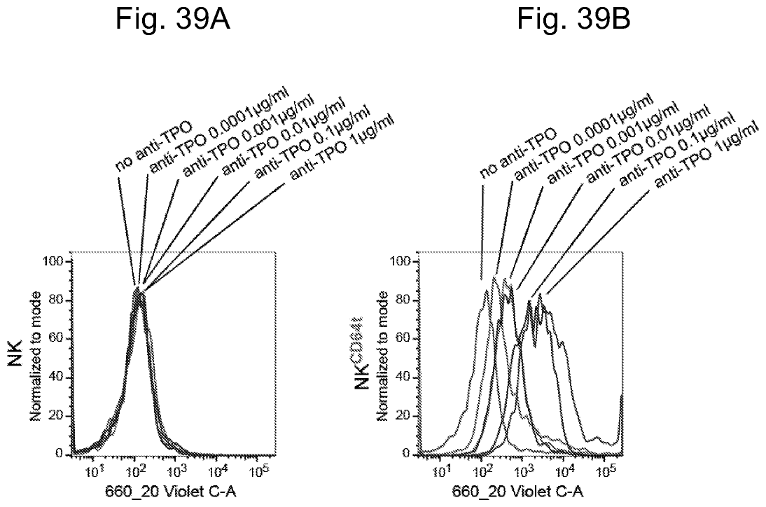

While NK cells did not bind any free IgG1 (using an anti-TPO antibody, FIG. 39A), NK^{CD64t} cells were able to capture and bind free IgG1 F_{c} (FIG. 39B).

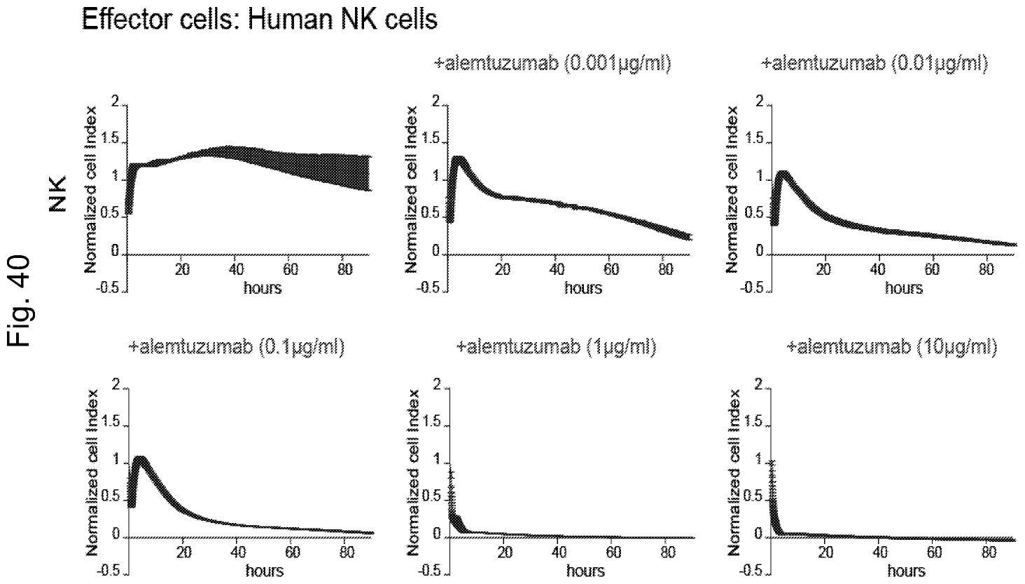

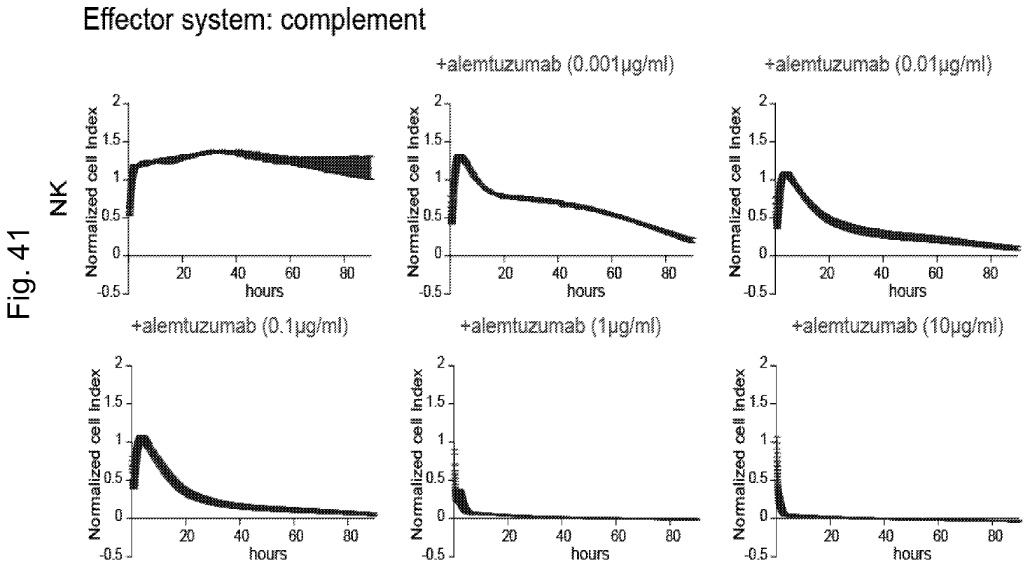

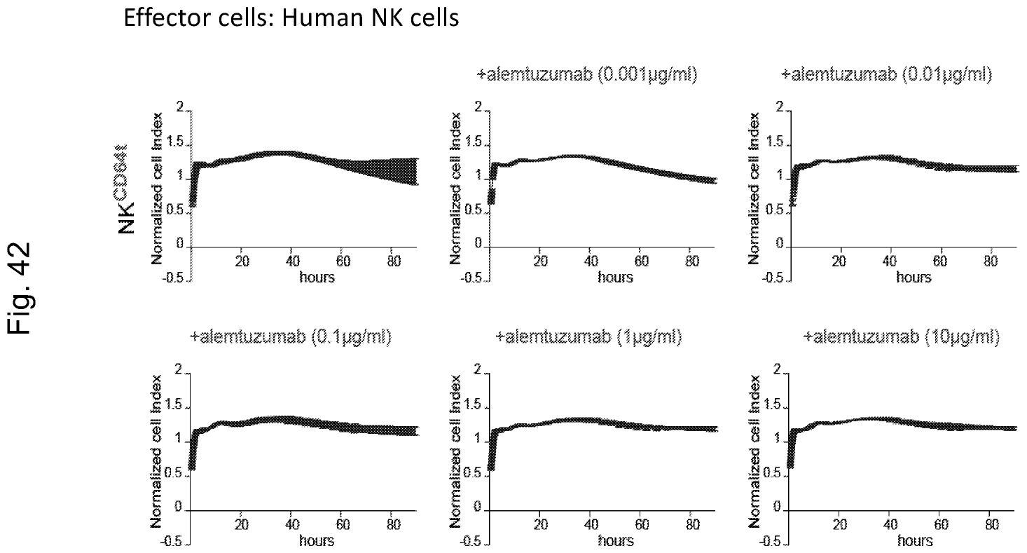

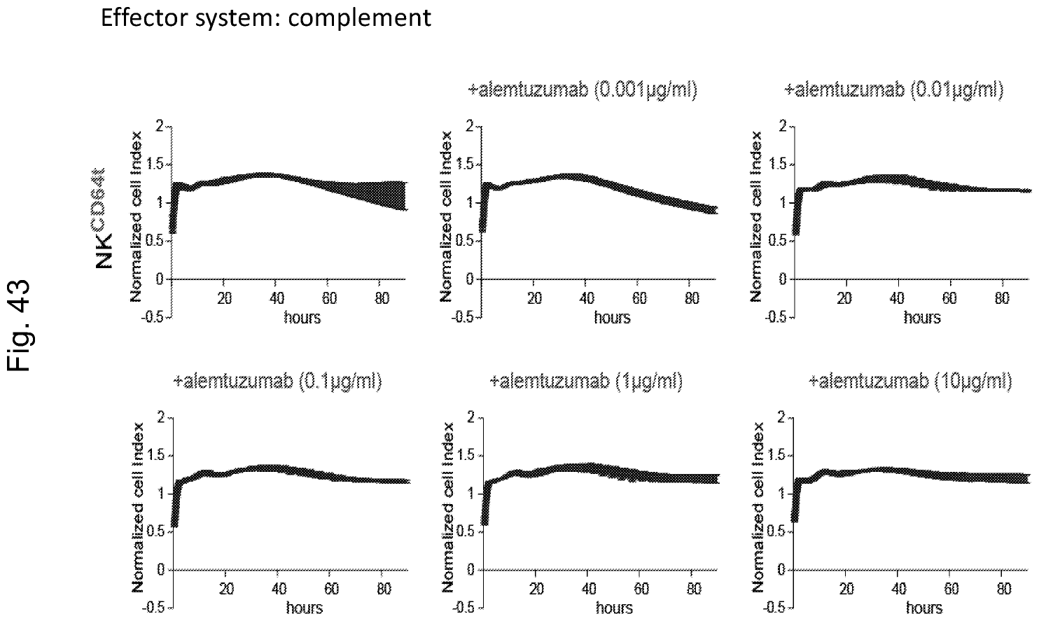

ADCC (FIG. 40) and CDC assays (FIG. 41) with the anti-CD52 antibody alemtuzumab were performed, and the NK cells were killed rapidly. NK^{CD64t} cells, however, were able to evade antibody-mediated killing in both ADCC (FIG. 42) and CDC assays (FIG. 43). CD64t expression makes NK cells resistant against antibody-mediated killing.

### VIII. Exemplary sequences

Human β-2-Microglobulin
SEQ ID NO: 1
MSRSVALAVLALLSLSGLEAIQRTPKIQVYSRHPAENGKSNELNC
YVSGFHPSDIEVDLLKNGERIEKVEHSDLSFSKDWSFYLLYYTEF
TPTEKDEYACRVNHVTLSQPKIVKWDRDI
Human CIITA protein, 160 amino acid N-terminus
SEQ ID NO: 2
MRCLAPRPAGSYLSEPQGSSQCATMELGPLEGGYLELLNSDADPL
CLYHFYDQMDLAGEEEIELYSEPDTDTINCDQFSRLLCDMEGDEE
TREAYANIAELDQYVFQDSQLEGLSKDIFKHIGPDEVIGESMEMP
AEVGQKSQKRPFPEELPADLKHWKP
Human CD47
SEQ ID NO: 3
MWPLVAALLLGSACCGSAQLLENKTKSVEFTFCNDTVVIPCFVTN
MEAQNTTEVYVKWKFKGRDIYTFDGALNKSTVPTDESSAKIEVSQ
LLKGDASLKMDKSDAVSHTGNYTCEVTELTREGETIIELKYRVVS
WESPNENILIVIFPIFAILLFWGQFGIKTLKYRSGGMDEKTIALL
VAGLVITVIVIVGAILFVPGEYSLKNATGLGLIVTSTGILILLHY
YVESTAIGLTSFVIAILVIQVIAYILAVVGLSLCIAACIPMHGPL
LISGLSILALAQLLGLVYMKFVE
Herpes Simplex Virus Thimidine Kinase (HSV-tk)
SEQ ID NO: 4
MASYPCHQHASAFDQAARSRGHSNRRTALRPRRQQEATEVRLEQK
MPTLLRVYIDGPHGMGKTTTTQLLVALGSRDDIVYVPEPMTYWQV
LGASETIANIYTTQHRLDQGEISAGDAAVVMTSAQITMGMPYAVT
DAVLAPHVGGEAGSSHAPPPALTLIFDRHPIAALLCYPAARYLMG
SMTPQAVLAFVALIPPTLPGTNIVLGALPEDRHIDRLAKRQRPGE
RLDLAMLAAIRRVYGLLANTVRYLQGGGSWWEDWGQLSGTAVPPQ
GAEPQSNAGPRPHIGDTLFTLFRAPELLAPNGDLYNVFAWALDVL
AKRLRPMHVFILDYDQSPAGCRDALLQLTSGMVQTHVTTPGSIPT
ICDLARTFAREMGEAN
Escherichia coli Cytosine Deaminase (EC-CD)
SEQ ID NO: 5
MSNNALQTIINARLPGEEGLWQIHLQDGKISAIDAQSGVMPITEN
SLDAEQGLVIPPEVEPHIHLDTTQTAGQPNWNQSGTLFEGIERWA
ERKALLTHDDVKQRAWQTLKWQIANGIQHVRTHVDVSDATLTALK
AMLEVKQEVAPWIDLQIVAFPQEGILSYPNGEALLEEALRLGADV
VGAIPHFEFTREYGVESLHKTFALAQKYDRLIDVHCDEIDDEQSR
EVETVAALAHHEGMGARVTASHTTAMHSYNGAYTSRLFRLLKMSG
INFVANPLVNIHLQGREDTYPKRRGITRVKEMLESGINVCFGHDD
VFDPWYPLGTANMLQVLHMGLHVCQLMGYGQINDGLNLITHHSAR
TLNLQDYGIAAGNSANLIILPAENGEDALRRQVPVRYSVRGGKVI
ASTQPAQTTVYLEQPEAIDYKR
Truncated human Caspase 9
SEQ ID NO: 6
GFGDVGALESLRGNADLAYILSMEPCGHCLIINNVNFCRESGLRT
RTGSNIDCEKLRRRESSLHFMVEVKGDLTAKKMVLALLELAQQDH
GALDCCVVVILSHGCQASHLQFPGAVYGTDGCPVSVEKIVNIFNG
TSCPSLGGKPKLFFIQACGGEQKDHGFEVASTSPEDESPGSNPEP
DATPFQEGLRTFDQLDAISSLPTPSDIFVSYSTFPGFVSWRDPKS
GSWYVETLDDIFEQWAHSEDLQSLLLRVANAVSVKGIYKQMPGCF
NFLRKKLFFKTS
Human CD64
NM_000566
SEQ ID NO: 7
MWFLTTLLLWVPVDGQVDTTKAVITLQPPWVSVFQEETVTLHCEV
LHLPGSSSTQWFLNGTATQTSTPSYRITSASVNDSGEYRCQRGLS
GRSDPIQLEIHRGWLLLQVSSRVFTEGEPLALRCHAWKDKLVYNV
LYYRNGKAFKFFHWNSNLTILKTNISHNGTYHCSGMGKHRYTSAG
ISVTVKELFPAPVLNASVTSPLLEGNLVTLSCETKLLLQRPGLQL
YFSFYMGSKTLRGRNTSSEYQILTARREDSGLYWCEAATEDGNVL
KRSPELELQVLGLQLPTPVWFHVLFYLAVGIMFLVNTVLWVTIRK
ELKRKKKWDLEISLDSGHEKKVISSLQEDRHLEEELKCQEQKEEQ
LQEGVHRKEPQGAT
Human CD52
NM_001803
SEQ ID NO: 8
MKRFLFLLLTISLLVMVQIQTGLSGQNDTSQTSSPSASSSMSGGI
FLFFVANAIIHLFCES
Human CD16 FCGR3A
NP_000560.6
SEQ ID NO: 9
MAEGTLWQILCVSSDAQPQTFEGVKGADPPTLPPGSFLPGPVLWW
GSLARLQTEKSDEVSRKGNWWVTEMGGGAGERLFTSSCLVGLVPL
GLRISLVTCPLQCGIMWQLLLPTALLLLVSAGMRTEDLPKAVVFL
EPQWYRVLEKDSVTLKCQGAYSPEDNSTQWFHNESLISSQASSYF
IDAATVDDSGEYRCQTNLSTLSDPVQLEVHIGWLLLQAPRWVEKE
EDPIHLRCHSWKNTALHKVTYLQNGKGRKYFHHNSDFYIPKATLK
DSGSYFCRGLFGSKNVSSETVNITITQGLAVSTISSFFPPGYQVS
FCLVMVLLFAVDTGLYFSVKTNIRSSTRDWKDHKFKWRKDPQDK
Human CD16 FCGR3B
NP_001231682.2
SEQ ID NO: 10
MWQLLLPTALLLLVSAGMRTEDLPKAVVFLEPQWYSVLEKDSVTL
KCQGAYSPEDNSTQWFHNENLISSQASSYFIDAATVNDSGEYRCQ
TNLSTLSDPVQLEVHIGWLLLQAPRWVEKEEDPIHLRCHSWKNTA
LHKVTYLQNGKDRKYFHHNSDFHIPKATLKDSGSYFCRGLVGSKN
VSSETVNITITQGLAVSTISSFSPPGYQVSFCLVMVLLFAVDTGL
YFSVKTNI
Human CD32 FCGR2A
>NP_001129691.1
SEQ ID NO: 11
MTMETQMSQNVCPRNLWLLQPLTVLLLLASADSQAAAPPKAVLKL
EPPWINVLQEDSVTLTCQGARSPESDSIQWFHNGNLIPTHTQPSY
RFKANNNDSGEYTCQTGQTSLSDPVHLTVLSEWLVLQTPHLEFQE
GETIMLRCHSWKDKPLVKVTFFQNGKSQKFSHLDPTESIPQANHS
HSGDYHCTGNIGYTLFSSKPVTITVQVPSMGSSSPMGIIVAVVIA
TAVAAIVAAVVALIYCRKKRISANSTDPVKAAQFEPPGRQMIAIR
KRQLEETNNDYETADGGYMTLNPRAPTDDDKNIYLTLPPNDHVNS
NN
Human CD32 FCGR2B
>NP_003992.3
SEQ ID NO: 12
MGILSFLPVLATESDWADCKSPQPWGHMLLWTAVLFLAPVAGTPA
APPKAVLKLEPQWINVLQEDSVTLTCRGTHSPESDSIQWFHNGNL
IPTHTQPSYRFKANNNDSGEYTCQTGQTSLSDPVHLTVLSEWLVL
QTPHLEFQEGETIVLRCHSWKDKPLVKVTFFQNGKSKKFSRSDPN
ESIPQANHSHSGDYHCTGNIGYTLYSSKPVTITVQAPSSSPMGII
VAVVTGIAVAAIVAAVVALIYCRKKRISALPGYPECREMGETLPE
KPANPTNPDEADKVGAENTITYSLLMHPDALEEPDDQNRI
Human CD32 FCGR2C
>NP_963857.3
SEQ ID NO: 13
MGILSFLPVLATESDWADCKSPQPWGHMLLWTAVLFLAPVAGTPA
APPKAVLKLEPQWINVLQEDSVTLTCRGTHSPESDSIPWFHNGNL
IPTHTQPSYRFKANNNDSGEYTCQTGQTSLSDPVHLTVLSEWLVL
QTPHLEFQEGETIVLRCHSWKDKPLVKVTFFQNGKSKKFSRSDPN
ESIPQANHSHSGDYHCTGNIGYTLYSSKPVTITVQAPSSSPMGII
VAVVTGIAVAAIVAAVVALIYCRKKRISANSTDPVKAAQFEPPGR
QMIAIRKRQPEETNNDYETADGGYMTLNPRAPTDDDKNIYLTLPP
NDHVNSNN
Truncated human CD16
SEQ ID NO: 14
MWQLLLPTALLLLVSAGMRTEDLPKAVVFLEPQWYRVLEKDSVTL
KCQGAYSPEDNSTQWFHNESLISSQASSYFIDAATVDDSGEYRCQ
TNLSTLSDPVQLEVHIGWLLLQAPRWVFKEEDPIHLRCHSWKNTA
LHKVTYLQNGKGRKYFHHNSDFYIPKATLKDSGSYFCRGLFGSKN
VSSETVNITITQGLAVSTISSFFPPGYQVSFCLVMVLLFAVDTGL
YFSV
Truncated human CD32
SEQ ID NO: 15
MTMETQMSQNVCPRNLWLLQPLTVLLLLASADSQAAAPPKAVLKL
EPPWINVLQEDSVTLTCQGARSPESDSIQWFHNGNLIPTHTQPSY
RFKANNNDSGEYTCQTGQTSLSDPVHLTVLSEWLVLQTPHLEFQE
GETIMLRCHSWKDKPLVKVTFFQNGKSQKFSHLDPTFSIPQANHS
HSGDYHCTGNIGYTLFSSKPVTITVQVPSMGSSSPMGIIVAVVIA
TAVAAIVAAVVALIY
Truncated human CD64
SEQ ID NO: 16
MWFLTTLLLWVPVDGQVDTTKAVITLQPPWVSVFQEETVTLHCEV
LHLPGSSSTQWELNGTATQTSTPSYRITSASVNDSGEYRCQRGLS
GRSDPIQLEIHRGWLLLQVSSRVFTEGEPLALRCHAWKDKLVYNV
LYYRNGKAFKFFHWNSNLTILKTNISHNGTYHCSGMGKHRYTSAG
ISVTVKELFPAPVLNASVTSPLLEGNLVTLSCETKLLLQRPGLQL
YFSFYMGSKTLRGRNTSSEYQILTARREDSGLYWCEAATEDGNVL
KRSPELELQVLGLQLPTPVWFHVLFYLAVGIMFLVNTVLWVTI
TfR1 cytoplasmic domain (positions 1-67)
SEQ ID NO: 17
MMDQARSAFSNLEGGEPLSYTRESLARQVDGDNSHVEMKLAVDEE
ENADNNTKANVTKPKRCSGSIC
TfR1 transmembrane domain (positions 68-88)
SEQ ID NO: 18
YGTIAVIVFFLIGFMIGYLGY
TfR1 extracellular domain (positions 89-760)
SEQ ID NO: 19
CKGVEPKTECERLAGTESPVREEPGEDFPAARRLYWDDLKRKLSE
KLDSTDFTGTIKLLNENSYVPREAGSQKDENLALYVENQFREFKL
SKVWRDQHFVKIQVKDSAQNSVIIVDKNGRLVYLVENPGGYVAYS
KAATVTGKLVHANFGTKKDFEDLYTPVNGSIVIVRAGKITFAEKV
ANAESLNAIGVLIYMDQTKFPIVNAELSFFGHAHLGTGDPYTPGF
PSENHTQFPPSRSSGLPNIPVQTISRAAAEKLEGNMEGDCPSDWK
TDSTCRMVTSESKNVKLTVSNVLKEIKILNIFGVIKGFVEPDHYV
VVGAQRDAWGPGAAKSGVGTALLLKLAQMESDMVLKDGFQPSRSI
IFASWSAGDEGSVGATEWLEGYLSSLHLKAFTYINLDKAVLGTSN
FKVSASPLLYTLIEKTMQNVKHPVTGQFLYQDSNWASKVEKLTLD
NAAFPFLAYSGIPAVSFCFCEDTDYPYLGTTMDTYKELIERIPEL
NKVARAAAEVAGQFVIKLTHDVELNLDYERYNSQLLSFVRDLNQY
RADIKEMGLSLQWLYSARGDFFRATSRLTTDEGNAEKTDRFVMKK
LNDRVMRVEYHELSPYVSPKESPFRHVFWGSGSHTLPALLENLKL
RKQNNGAFNETLERNQLALATWTIQGAANALSGDVWDIDNEF
TfR2 cytoplasmic domain (positions 1-83)
SEQ ID NO: 20
MERLWGLFQRAQQLSPRSSQTVYQRVEGPRKGHLEEEEEDGEEGA
ETLAHFCPMELRGPEPLGSRPRQPNLIPWAAAGRRAAP
TfR2 transmembrane domain (positions 84-104)
SEQ ID NO: 21
YLVLTALLIFTGAFLLGYVAF
TfR2 extracellular domain (positions 105-801)
SEQ ID NO: 22
RGSCQACGDSVLVVSEDVNYEPDLDFHQGRLYWSDLQAMFLQFLG
EGRLEDTIRQTSLRERVAGSAGMAALTQDIRAALSRQKLDHVWTD
THYVGLQFPDPAHPNTLHWVDEAGKVGEQLPLEDPDVYCPYSAIG
NVTGELVYAHYGRPEDLQDLRARGVDPVGRLLLVRVGVISFAQKV
TNAQDFGAQGVLIYPEPADESQDPPKPSLSSQQAVYGHVHLGTGD
PYTPGFPSFNQTQFPPVASSGLPSIPAQPISADIASRLLRKLKGP
VAPQEWQGSLLGSPYHLGPGPRLRLVVNNHRTSTPINNIFGCIEG
RSEPDHYVVIGAQRDAWGPGAAKSAVGTAILLELVRTFSSMVSNG
FRPRRSLLFISWDGGDFGSVGSTEWLEGYLSVLHLKAVVYVSLDN
AVLGDDKFHAKTSPLLTSLIESVLKQVDSPNHSGQTLYEQVVFTN
PSWDAEVIRPLPMDSSAYSFTAFVGVPAVEFSFMEDDQAYPFLHT
KEDTYENLHKVLQGRLPAVAQAVAQLAGQLLIRLSHDRLLPLDFG
RYGDVVLRHIGNLNEFSGDLKARGLTLQWVYSARGDYIRAAEKLR
QEIYSSEERDERLTRMYNVRIMRVEFYFLSQYVSPADSPFRHIFM
GRGDHTLGALLDHLRLLRSNSSGTPGATSSTGFQESRFRRQLALL
TWTLQGAANALSGDVWNIDNNE

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

What is claimed:

1. A modified cell, wherein said modified cell expresses an F_{c} receptor protein comprising a truncation or modification, wherein said F_{c} receptor protein expression causes said modified cell to be less susceptible to antibody dependent cellular cytoxicity (ADCC) or complement-dependent cytotoxicity (CDC), wherein said truncation or modification reduces or eliminates said F_{c} receptor intracellular signaling.

2. The modified cell of claim 1, wherein said F_{c} receptor protein is selected from the group consisting of a truncated CD16 (CD16t), truncated CD32 (CD32t), and truncated CD64 (CD64t).

3. The modified cell of claim 1, wherein said cell is a pluripotent cell.

4. (canceled)

5. The modified cell of claim 4, wherein said CD64t protein has the sequence of SEQ ID NO:16.

6. The modified cell of claim 1, wherein said modified cell is derived from a human hypo-immunogenic pluripotent (HIP) cell or a human hypo-immunogenic pluripotent ABO blood group O Rhesus Factor negative (HIPO−) cell.

7-10. (canceled)

11. The modified cell of claim 1, further comprising a suicide gene that is activated by a trigger that causes said modified cell to die.

12-21. (canceled)

22. The modified cell of claim 1, wherein said cell is selected from the group consisting of a chimeric antigen receptor (CAR) cell, a T cell, an NK cell, an ILC, an endothelial cell, a dopaminergic neuron, a neuroglial cell, a pancreatic islet cell, a pancreatic beta cell, a thyroid cell, an endocrine cell, a fibroblast, a hepatocyte, a cardiomyocyte, and a retinal pigment endothelium cell.

23-26. (canceled)

27. A method of treating a disease, comprising administering the modified cell of claim 1 to a subject.

28. The method of claim 27, wherein said modified cell is selected from the group consisting of a chimeric antigen receptor (CAR) cell, a T cell, an NK cell, an ILC, an endothelial cell, a dopaminergic neuron, a neuroglial cell, a pancreatic islet cell, a pancreatic beta cell, a thyroid cell, a fibroblast, a hepatocyte, a cardiomyocyte, and a retinal pigment endothelium cell.

29. The method of claim 27, wherein said disease is selected from the group consisting of Type I Diabetes, a cardiac disease, a neurological disease, an endocrine disease, a cancer, an ocular disease, and a vascular disease.

30. A method for generating the modified cell of claim 1, comprising expressing said CD16t, CD32t, or CD64t protein in a parental non-modified version of said cell.

31. (canceled)

32. The method of claim 30, wherein said modified cell is derived from a HIP cell, a HIPO− cell, an iPSC cell, or an ESC cell.

33-37. (canceled)

38. A pharmaceutical composition for treating a disease, comprising a cell derived from the modified cell of claim 1 and a pharmaceutically-acceptable carrier.

39. The pharmaceutical composition of claim 38, wherein said cell is selected from the group consisting of a chimeric antigen receptor (CAR) cell, a T cell, an NK cell, an ILC, an endothelial cell, a dopaminergic neuron, a neuroglial cell, a pancreatic islet cell, a pancreatic beta cell, a thyroid cell, a fibroblast, a hepatocyte, a cardiomyocyte, and a retinal pigment endothelium cell.

40-45. (canceled)

46. The modified cell of claim 1, wherein said cell comprises an Fc receptor chimera comprising a cytoplasmic domain that does not mediate an Fc receptor signaling pathway and wherein said cytoplasmic domain promotes endocytosis of an antibody bound by its Fc to an extracellular domain of said Fc receptor chimera.

47. The modified cell of claim 46, wherein said cytoplasmic domain is from a transferrin receptor.

48. (canceled)

49. The modified cell of any one of claims 46-48, wherein said F_{c} receptor chimera comprises a CD16, CD32, or CD64 cell surface domain and a TfR1 or TfR2 cytoplasmic domain.

50. The modified cell of claim 1, wherein said cell expresses a recombinant SIRPα engager protein.

51. The modified cell of claim 50, wherein said SIRPα engager protein comprises an immunoglobulin superfamily domain, an antibody Fab domain, or a single chain variable fragment (scFV).

52. (canceled)

53. The modified cell of claim 22, wherein said cell comprises a B2M−/− phenotype, a CIITA−/− phenotype, a CD64t, and a SIRPα-engager molecule.

54. The modified cell of claim 53, wherein said SIRPα-engager protein is CD47.

55. The modified cell of claim 53, wherein said cell is an engineered NK cell.


SEQUENCE LISTING (23 sequences)

SEQ ID NO: 1 - protein, 119 aa, Homo sapiens [sequence omitted]

SEQ ID NO: 2 - protein, 160 aa, Homo sapiens
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKP

SEQ ID NO: 3 - protein, 293 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 4 - protein, 376 aa, Human alphaherpesvirus 1
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 5 - protein, 427 aa, Escherichia coli
MSNNALQTIINARLPGEEGLWQIHLQDGKISAIDAQSGVMPITENSLDAEQGLVIPPFVEPHIHLDTTQTAGQPNWNQSGTLFEGIERWAERKALLTHDDVKQRAWQTLKWQIANGIQHVRTHVDVSDATLTALKAMLEVKQEVAPWIDLQIVAFPQEGILSYPNGEALLEEALRLGADVVGAIPHFEFTREYGVESLHKTFALAQKYDRLIDVHCDEIDDEQSRFVETVAALAHHEGMGARVTASHTTAMHSYNGAYTSRLFRLLKMSGINFVANPLVNIHLQGRFDTYPKRRGITRVKEMLESGINVCFGHDDVFDPWYPLGTANMLQVLHMGLHVCQLMGYGQINDGLNLITHHSARTLNLQDYGIAAGNSANLIILPAENGFDALRRQVPVRYSVRGGKVIASTQPAQTTVYLEQPEAIDYKR

SEQ ID NO: 6 - protein, 282 aa, Homo sapiens
GFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 7 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 8 - protein, 61 aa, Homo sapiens
MKRFLFLLLTISLLVMVQIQTGLSGQNDTSQTSSPSASSSMSGGIFLFFVANAIIHLFCFS

SEQ ID NO: 9 - protein, 359 aa, Homo sapiens
MAEGTLWQILCVSSDAQPQTFEGVKGADPPTLPPGSFLPGPVLWWGSLARLQTEKSDEVSRKGNWWVTEMGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 10 - protein, 233 aa, Homo sapiens
MWQLLLPTALLLLVSAGMRTEDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 11 - protein, 317 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 12 - protein, 310 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 13 - protein, 323 aa, Homo sapiens
MGILSFLPVLATESDWADCKSPQPWGHMLLWTAVLFLAPVAGTPAAPPKAVLKLEPQWINVLQEDSVTLTCRGTHSPESDSIPWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANSTDPVKAAQFEPPGRQMIAIRKRQPEETNNDYETADGGYMTLNPRAPTDDDKNIYLTLPPNDHVNSNN

SEQ ID NO: 14 - protein, 229 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSV

SEQ ID NO: 15 - protein, 240 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIY

SEQ ID NO: 16 - protein, 313 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTI

SEQ ID NO: 17 - protein, 67 aa, Unknown
  Description of Unknown TfR1 cytoplasmic domain sequence
MMDQARSAFSNLFGGEPLSYTRFSLARQVDGDNSHVEMKLAVDEEENADNNTKANVTKPKRCSGSIC

SEQ ID NO: 18 - protein, 21 aa, Unknown
  Description of Unknown TfR1 transmembrane domain sequence
YGTIAVIVFFLIGFMIGYLGY

SEQ ID NO: 19 - protein, 672 aa, Unknown
  Description of Unknown TfR1 extracellular domain sequence
CKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 20 - protein, 83 aa, Unknown
  Description of Unknown TfR2 cytoplasmic domain sequence
MERLWGLFQRAQQLSPRSSQTVYQRVEGPRKGHLEEEEEDGEEGAETLAHFCPMELRGPEPLGSRPRQPNLIPWAAAGRRAAP

SEQ ID NO: 21 - protein, 21 aa, Unknown
  Description of Unknown TfR2 transmembrane domain sequence
YLVLTALLIFTGAFLLGYVAF

SEQ ID NO: 22 - protein, 697 aa, Unknown
  Description of Unknown TfR2 extracellular domain sequence
RGSCQACGDSVLVVSEDVNYEPDLDFHQGRLYWSDLQAMFLQFLGEGRLEDTIRQTSLRERVAGSAGMAALTQDIRAALSRQKLDHVWTDTHYVGLQFPDPAHPNTLHWVDEAGKVGEQLPLEDPDVYCPYSAIGNVTGELVYAHYGRPEDLQDLRARGVDPVGRLLLVRVGVISFAQKVTNAQDFGAQGVLIYPEPADFSQDPPKPSLSSQQAVYGHVHLGTGDPYTPGFPSFNQTQFPPVASSGLPSIPAQPISADIASRLLRKLKGPVAPQEWQGSLLGSPYHLGPGPRLRLVVNNHRTSTPINNIFGCIEGRSEPDHYVVIGAQRDAWGPGAAKSAVGTAILLELVRTFSSMVSNGFRPRRSLLFISWDGGDFGSVGSTEWLEGYLSVLHLKAVVYVSLDNAVLGDDKFHAKTSPLLTSLIESVLKQVDSPNHSGQTLYEQVVFTNPSWDAEVIRPLPMDSSAYSFTAFVGVPAVEFSFMEDDQAYPFLHTKEDTYENLHKVLQGRLPAVAQAVAQLAGQLLIRLSHDRLLPLDFGRYGDVVLRHIGNLNEFSGDLKARGLTLQWVYSARGDYIRAAEKLRQEIYSSEERDERLTRMYNVRIMRVEFYFLSQYVSPADSPFRHIFMGRGDHTLGALLDHLRLLRSNSSGTPGATSSTGFQESRFRRQLALLTWTLQGAANALSGDVWNIDNNF

SEQ ID NO: 23 - protein, 61 aa, Homo sapiens
RKELKRKKKWDLEISLDSGHEKKVISSLQEDRHLEEELKCQEQKEEQLQEGVHRKEPQGAT